(19) United States
(12) Patent Application Publication
(10) Pub. No.: US 2024/0002386 A1
(43) Pub. Date: Jan. 4, 2024

(54) SOLID FORMS OF 4-(5-(4-FLUOROPHENYL)-6-(TETRAHYDRO-2H-PYRAN-4-YL)-1,5-DIHYDROPYRROLO[2,3-F]INDAZOL-7-YL)BENZOIC ACID

(71) Applicant: Vertex Pharmaceuticals Incorporated, Boston, MA (US)

(72) Inventors: Yi SHI, Natick, MA (US); Mei-Hsiu LAI, Waltham, MA (US); Ales MEDEK, Winchester, MA (US); Kan-Nian HU, Brighton, MA (US); Zhengtian SONG, (CN); Elisa A. TORRICO GUZMAN, Natick, MA (US); Kathleen Paige SOKOLOWSKY, Boston, MA (US); Simon GIROUX, Cambridge, MA (US); Siying LIU, Malden, MA (US); Kirk Alan OVERHOFF, Lynn, MA (US); Setu RODAY, Arlington, MA (US); Rupa SAWANT, Wayland, MA (US); Marisa SPOSATO, Cambridge, MA (US)

(21) Appl. No.: 18/037,121

(22) PCT Filed: Nov. 17, 2021
(86) PCT No.: PCT/US2021/072451

§ 371 Date: May 16, 2023

Related U.S. Application Data
(60) Provisional application No. 63/114,742, filed on Nov. 17, 2020.

(51) Int. Cl. C07D 487/04; C07D 471/14

(52) U.S. Cl. CPC C07D 487/04; C07D 471/14; C07B 2200/13

(57) ABSTRACT

Solid forms of 4-(5-(4-fluorophenyl)-6-(tetrahydro-2H-pyran-4-yl)-1,5-dihydropyrrolo[2,3-f]indazol-7-yl)benzoic acid (Compound 1) capable of modulating alpha-1 antitrypsin (AAT) activity and methods of treating alpha-1 antitrypsin deficiency (AATD) by administering one or more such forms, and methods of using and preparing the same for treatment of alpha-1 antitrypsin deficiency (AATD).

This application claims the benefit of priority of U.S. Provisional Application No. 63/114,742, filed Nov. 17, 2020, the contents of which are herein incorporated by reference.

The disclosure provides solid forms of 4-(5-(4-fluorophenyl)-6-(tetrahydro-2H-pyran-4-yl)-1,5-dihydropyrrolo[2,3-f]indazol-7-yl)benzoic acid (Compound 1) that are capable of modulating alpha-1 antitrypsin (AAT) activity and methods of treating alpha-1 antitrypsin deficiency (AATD) by administering one or more such forms.

AATD is a genetic disorder characterized by low circulating levels of AAT. While treatments for AATD exist, there is currently no cure. AAT is produced primarily in liver cells and secreted into the blood, but it is also made by other cell types including lung epithelial cells and certain white blood cells. AAT inhibits several serine proteases secreted by inflammatory cells (most notably neutrophil elastase [NE], proteinase 3, and cathepsin G) and thus protects organs such as the lung from protease-induced damage, especially during periods of inflammation.

The mutation most commonly associated with AATD involves a substitution of lysine for glutamic acid (E342K) in the SERPINA1 gene that encodes the AAT protein. This mutation, known as the Z mutation or the Z allele, leads to misfolding of the translated protein, which is therefore not secreted into the bloodstream and can polymerize within the producing cell. Consequently, circulating AAT levels in individuals homozygous for the Z allele (PiZZ) are markedly reduced; only approximately 15% of mutant Z-AAT protein folds correctly and is secreted by the cell. An additional consequence of the Z mutation is that the secreted Z-AAT has reduced activity compared to wild-type protein, with 40% to 80% of normal antiprotease activity (American thoracic society/European respiratory society, Am J Respir Crit Care Med. 2003; 168(7):818-900; and Ogushi et al. J Clin Invest. 1987; 80(5):1366-74.)

The accumulation of polymerized Z-AAT protein within hepatocytes results in a gain-of-function cytotoxicity that can result in cirrhosis or liver cancer later in life and neonatal liver disease in 12% of patients. This accumulation may spontaneously remit but can be fatal in a small number of children. The deficiency of circulating AAT results in unregulated protease activity that degrades lung tissue over time, resulting in emphysema, a form of chronic obstructive pulmonary disease (COPD). This effect is severe in PiZZ individuals and typically manifests in middle age, resulting in a decline in quality of life and shortened lifespan (mean 68 years of age) (Tanash et al. Int J Chron Obstruct Pulm Dis. 2016; 11:1663-9). The effect is more pronounced in PiZZ individuals who smoke, resulting in an even further shortened lifespan (58 years). (Piitulainen and Tanash, COPD 2015; 12(1):36-41.) PiZZ individuals account for the majority of those with clinically relevant AATD lung disease. Accordingly, there is a need for additional and effective treatments for AATD.

A milder form of AATD is associated with the SZ genotype in which the Z-allele is combined with an S-allele. The S allele is associated with somewhat reduced levels of circulating AAT but causes no cytotoxicity in liver cells. The result is clinically significant lung disease but not liver disease. (Fregonese and Stolk, Orphanet J Rare Dis. 2008; 33:16.) As with the ZZ genotype, the deficiency of circulating AAT in subjects with the SZ genotype results in unregulated protease activity that degrades lung tissue over time and can result in emphysema, particularly in smokers.

The current standard of care for AAT deficient individuals who have or show signs of developing significant lung or liver disease is augmentation therapy or protein replacement therapy. Augmentation therapy involves administration of a human AAT protein concentrate purified from pooled donor plasma to augment the missing AAT. Although infusions of the plasma protein have been shown to improve survival or slow the rate of emphysema progression, augmentation therapy is often not sufficient under challenging conditions such as during an active lung infection. Similarly, although protein replacement therapy shows promise in delaying progression of disease, augmentation does not restore the normal physiological regulation of AAT in patients and efficacy has been difficult to demonstrate. In addition, augmentation therapy requires weekly visits for treatment and augmentation therapy cannot address liver disease, which is driven by the toxic gain-of-function of the Z allele. Thus, there is a continuing need for new and more effective treatments for AATD.

4-(5-(4-fluorophenyl)-6-(tetrahydro-2H-pyran-4-yl)-1,5-dihydropyrrolo[2,3-f]indazol-7-yl)benzoic acid or Compound 1 is disclosed in International Patent Application No. PCT/US2020/032832 (incorporated herein by reference in its entirety), published as International Publication No. WO 2020/247160, as a potent modulator of AAT activity for treatment of AATD:

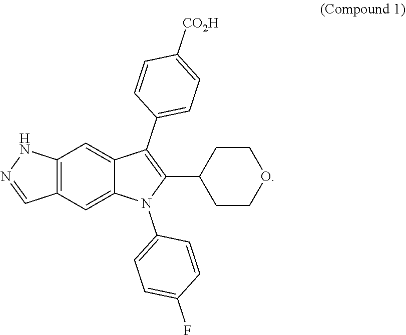 [SMILES: O=C(O)c1ccc(-c2c(C3CCOCC3)n(-c3ccc(F)cc3)c3cc4cn[nH]c4cc23)cc1]

In some embodiments, the solid form of Compound 1 is a neat Form C.

In some embodiments, the solid form of Compound 1 is a salt of Compound 1. In some embodiments, the solid form of Compound 1 is a Na salt of Compound 1. In some embodiments, the solid form of Compound 1 is a Na salt Form A. In some embodiments, the solid form of Compound 1 is a Na salt Form B. In some embodiments, the solid form of Compound 1 is a Na salt Form C. In some embodiments, the solid form of Compound 1 is a Na salt Form D.

In some embodiments, the solid form of Compound 1 is a Ca salt of Compound 1. In some embodiments, the solid form of Compound 1 is a Ca salt Form A.

In some embodiments, the solid form of Compound 1 is a HCl salt of Compound 1. In some embodiments, the solid form of Compound 1 is a HCl salt Form A.

In some embodiments, the solid form of Compound 1 is a solvate of Compound 1. In some embodiments, the solid form of Compound 1 is a DMSO solvate of Compound 1. In some embodiments, the solid form of Compound 1 is a DMSO solvate Form A.

In some embodiments, the solid form of Compound 1 is a EtOH solvate of Compound 1. In some embodiments, the solid form of Compound 1 is a EtOH solvate Form A.

In some embodiments, the solid form of Compound 1 is a salt or cocrystal of Compound 1. In some embodiments, the solid form of Compound 1 is a tartrate salt or cocrystal of Compound 1. In some embodiments, the solid form of Compound 1 is a tartrate salt or cocrystal Form A. In some embodiments, the solid form of Compound 1 is a tartrate salt or cocrystal Form B. In some embodiments, the solid form of Compound 1 is a tartrate salt or cocrystal Form C. In some embodiments, the solid form of Compound 1 is a tartrate salt or cocrystal Form D.

In some embodiments, the solid form of Compound 1 is a solid dispersion comprising a solid form of Compound 1 or a salt, or a solvate, or a cocrystal thereof, and a polymer carrier. In some embodiments, the solid dispersion is a spray-dried dispersion comprising a solid form of Compound 1 or a salt, or a solvate, or a cocrystal thereof, and a polymer carrier. In some embodiments, the solid dispersion comprises one or more polymers. In some embodiments, the one or more polymers in the solid dispersion is selected from pyrrolidones, celluloses, poloxamers, polymethacrylate based copolymers, and triblock copolymers. In some embodiments, the solid dispersion comprising amorphous Compound 1 also comprises HPMCAS.

Another aspect of the disclosure provides methods of treating AATD comprising administering to a subject in need thereof, at least one solid form of Compound 1 or a pharmaceutical composition comprising the at least one solid form of Compound 1.

In some embodiments, the methods of treatment include administration of at least one additional active agent to the subject in need thereof, either in the same pharmaceutical composition as the at least one solid form of Compound 1 or as separate compositions. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one solid form Compound 1, or as separate compositions, wherein the additional active agent is alpha-1 antitrypsin protein (AAT) from the blood plasma of healthy human donors.

In some embodiments, the methods of treatment include administration of at least one additional active agent to the subject in need thereof, either in the same pharmaceutical composition as the at least one solid form of Compound 1, or as separate compositions, wherein the additional active agent is recombinant AAT.

### BRIEF DESCRIPTION OF THE DRAWINGS

FIG. 1A shows an XRPD diffractogram of Compound 1 neat Form C.

FIG. 1B shows a solid state ¹⁹F NMR spectrum of Compound 1 neat Form C.

FIG. 1C shows a TGA thermogram of Compound 1 neat Form C.

FIG. 1D shows a DSC thermogram of Compound 1 neat Form C.

FIG. 2A shows an XRPD diffractogram of Compound 1 Na salt Form A.

FIG. 2B shows a TGA thermogram of Compound 1 Na salt Form A.

FIG. 2C shows a DSC thermogram of Compound 1 Na salt Form A.

FIG. 3A shows an XRPD diffractogram of Compound 1 Na salt Form B.

FIG. 3B shows a TGA thermogram of Compound 1 Na salt Form B.

FIG. 3C shows a DSC thermogram of Compound 1 Na salt Form B.

FIG. 4A shows an XRPD diffractogram of Compound 1 Na salt Form C.

FIG. 4B shows a solid state ¹³C NMR spectrum of Compound 1 Na salt Form C.

FIG. 4C shows a solid state ²³Na NMR spectrum of Compound 1 Na salt Form C.

FIG. 4D shows a TGA thermogram of Compound 1 Na salt Form C.

FIG. 4E shows a DSC thermogram of Compound 1 Na salt Form C.

FIG. 5A shows an XRPD diffractogram of Compound 1 Na salt Form D.

FIG. 5B shows a solid state ¹³C NMR spectrum of Compound 1 Na salt Form D.

FIG. 5C shows a solid state ²³Na NMR spectrum of Compound 1 Na salt Form D.

FIG. 6A shows an XRPD diffractogram of Compound 1 Ca salt Form A.

FIG. 6B shows a TGA thermogram of Compound 1 Ca salt Form A.

FIG. 6C shows a DSC thermogram of Compound 1 Ca salt Form A.

FIG. 7A shows an XRPD diffractogram of Compound 1 HCl salt Form A.

FIG. 7B shows a TGA thermogram of Compound 1 HCl salt Form A.

FIG. 7C shows a DSC thermogram of Compound 1 HCl salt Form A.

FIG. 8A shows an XRPD diffractogram of Compound 1 DMSO solvate Form A.

FIG. 8B shows a TGA thermogram of Compound 1 DMSO solvate Form A.

FIG. 8C shows a DSC thermogram of Compound 1 DMSO solvate Form A.

FIG. 9A shows an XRPD diffractogram of Compound 1 EtOH solvate Form A.

FIG. 9B shows a solid state ¹³C NMR spectrum of Compound 1 EtOH solvate Form A.

FIG. 9C shows a TGA thermogram of Compound 1 EtOH solvate Form A.

FIG. 9D shows a DSC thermogram of Compound 1 EtOH solvate Form A.

FIG. 10A shows an XRPD diffractogram of Compound 1 tartrate salt or cocrystal Form A.

FIG. 10B shows a TGA thermogram of Compound 1 tartrate salt or cocrystal Form A.

FIG. 10C shows a DSC thermogram of Compound 1 tartrate salt or cocrystal Form A.

FIG. 11A shows an XRPD diffractogram of Compound 1 tartrate salt or cocrystal Form B.

FIG. 11B shows a TGA thermogram of Compound 1 tartrate salt or cocrystal Form B.

FIG. 11C shows a DSC thermogram of Compound 1 tartrate salt or cocrystal Form B.

FIG. 12A shows an XRPD diffractogram of Compound 1 tartrate salt or cocrystal Form C.

FIG. 12B shows a TGA thermogram of Compound 1 tartrate salt or cocrystal Form C.

FIG. 12C shows a DSC thermogram of Compound 1 tartrate salt or cocrystal Form C.

FIG. 13 shows an XRPD diffractogram of Compound 1 tartrate salt or cocrystal Form D.

### DETAILED DESCRIPTION

### I. Definitions

The term “AAT” as used herein means alpha-1 antitrypsin or a mutation thereof, including, but not limited to, the AAT gene mutations such as Z mutations. As used herein, “Z-AAT” means AAT mutants which have the Z mutation.

As used herein, “mutations” can refer to mutations in the SERPINA1 gene (the gene encoding AAT) or the effect of alterations in the gene sequence on the AAT protein. A “SERPINA1 gene mutation” refers to a mutation in the SERPINA1 gene, and an “AAT protein mutation” refers to a mutation that results in an alteration in the amino acid sequence of the AAT protein. A genetic defect or mutation, or a change in the nucleotides in a gene in general, results in a mutation in the AAT protein translated from that gene.

As used herein, a patient who is “homozygous” for a particular gene mutation has the same mutation on each allele.

As used herein, a patient who has the PiZZ genotype is a patient who is homozygous for the Z mutation in the AAT protein.

The term “AATD” as used herein means alpha-1 antitrypsin deficiency, which is a genetic disorder characterized by low circulating levels of AAT.

The terms “patient” and “subject” are used interchangeably and refer to an animal, including a human.

The terms “effective dose” and “effective amount” are used interchangeably herein and refer to that amount of a compound that produces the desired effect for which it is administered (e.g., improvement in AATD or a symptom of AATD, lessening the severity of AATD or a symptom of AATD, and/or reducing the rate of onset or incidence of AATD or a symptom of AATD). The exact amount of an effective dose will depend on the purpose of the treatment, and will be ascertainable by one skilled in the art using known techniques (see, e.g., Lloyd (1999) The Art, Science and Technology of Pharmaceutical Compounding).

As used herein, the term “treatment” and its cognates refer to improving AATD or its symptoms in a subject, delaying the onset of AATD or its symptoms in a subject, or lessening the severity of AATD or its symptoms in a subject. “Treatment” and its cognates as used herein, include, but are not limited to the following: improved liver and/or spleen function, lessened jaundice, improved lung function, lessened lung diseases and/or pulmonary exacerbations (e.g., emphysema), lessened skin disease (e.g., necrotizing panniculitis), increased growth in children, improved appetite, and reduced fatigue. Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. In some embodiments, the term “about” refers to a variation of a stated numerical value of up to 10%, up to 5%, or up to 2%. Thus, e.g., in some embodiments, “about 10” means 10±1, 10±0.5, or 10±0.2.

Any one or more of the solid forms of Compound 1 may be administered once daily, twice daily, or three times daily for the treatment of AATD. In some embodiments, at least one solid form of Compound 1 is administered once daily. In some embodiments, at least one solid form of Compound 1 is administered twice daily. In some embodiments, at least one solid form of Compound 1 is administered three times daily.

In some embodiments, 10 mg to 1,500 mg, 100 mg to 1800 mg, 100 mg to 500 mg, 200 mg to 600 mg, 200 mg to 800 mg, 400 mg to 2,000 mg, 400 mg to 2,500 mg, or 400 mg to 600 mg of a compound of a solid form of Compound 1 is administered once daily, twice daily, or three times daily.

Any one or more of the solid forms of Compound 1 may be administered in combination with AAT augmentation therapy or AAT replacement therapy for the treatment of AATD.

As used herein, “AAT augmentation therapy” refers to the use of alpha-1 antitrypsin protein (AAT) from the blood plasma of healthy human donors to augment (increase) the alpha-1 antitrypsin levels circulating in the blood. “AAT replacement therapy” refers to administration of recombinant AAT.

As used herein, the term “ambient conditions” means room temperature, open air condition and uncontrolled humidity condition.

As used herein, the terms “crystalline form” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, solid state nuclear magnetic resonance (ssNMR), differential scanning calorimetry (DSC), dynamic vapor sorption (DVS), and/or thermogravimetric analysis (TGA). Accordingly, as used herein, the terms “crystalline Form [X] of Compound ([Y])” and “crystalline Form [C] of a [pharmaceutically acceptable] salt of Compound ([Y])” refer to unique crystalline forms that can be identified and distinguished from each other by one or more characterization techniques including, for example, X-ray powder diffraction (XRPD), single crystal X-ray diffraction, ssNMR, differential scanning calorimetry (DSC), dynamic vapor sorption (DVS), and/or thermogravimetric analysis (TGA). In some embodiments, the novel crystalline forms are characterized by an X-ray powder diffractogram having one or more signals at one or more specified two-theta values (° 2θ).

As used herein, the term “solvate” refers to a crystal form comprising one or more molecules of a compound of the present disclosure and, incorporated into the crystal lattice, one or more molecules of a solvent or solvents in stoichiometric or nonstoichiometric amounts. When the solvent is water, the solvate is referred to as a “hydrate”.

As used herein, the term “cocrystal” is a crystalline material composed of two or more different molecules, typically the compound and cocrystal formers (or coformers), in the same crystal lattice. Cocrystal components are in a neutral state and interact nonionically.

As used herein, the term “ssNMR” refers to the analytical characterization method of solid state nuclear magnetic resonance. ssNMR spectra can be recorded at ambient conditions on any magnetically active isotope present in the sample. The typical examples of active isotopes for small molecule active pharmaceutical ingredients include ¹H, ²H, ¹³C, ¹⁹F, ³¹P, ¹⁵N, ¹⁴N, ³⁵Cl, ¹¹B, ⁷Li, ¹⁷O, ²³Na, ⁷⁹Br, and ¹⁹⁵Pt.

As used herein, the term “XRPD” refers to the analytical characterization method of X-ray powder diffraction. XRPD patterns can be recorded at ambient conditions in transmission or reflection geometry using a diffractometer.

As used herein, the terms “X-ray powder diffractogram,” “X-ray powder diffraction pattern,” and “XRPD pattern” interchangeably refer to an experimentally obtained pattern plotting signal positions (on the abscissa) versus signal intensities (on the ordinate). For an amorphous material, an X-ray powder diffractogram may include one or more broad signals; and for a crystalline material, an X-ray powder diffractogram may include one or more signals, each identified by its angular value as measured in degrees 2θ (° 2θ), depicted on the abscissa of an X-ray powder diffractogram, which may be expressed as “a signal at . . . degrees two-theta,” “a signal at [a] two-theta value(s) of . . . ” and/or “a signal at at least . . . two-theta value(s) chosen from . . . .”

A “signal” or “peak” as used herein refers to a point in the XRPD pattern where the intensity as measured in counts is at a local maximum. One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

As used herein, “a signal at . . . degrees two-theta,” “a signal at [a] two-theta value[ ] of . . . ” and/or “a signal at at least . . . two-theta value(s) chosen from . . . ” refer to X-ray reflection positions as measured and observed in X-ray powder diffraction experiments (° 2θ).

The repeatability of the angular values is in the range of 0.2° 2θ, i.e., the angular value can be at the recited angular value+0.2 degrees two-theta, the angular value−0.2 degrees two-theta, or any value between those two end points (angular value+0.2 degrees two-theta and angular value −0.2 degrees two-theta).

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

The term “X-ray powder diffractogram having a signal at . . . two-theta values” as used herein refers to an XRPD pattern that contains X-ray reflection positions as measured and observed in X-ray powder diffraction experiments (° 2θ).

As used herein, the term “amorphous” refers to a solid material having no long range order in the position of its molecules. Amorphous solids are generally supercooled liquids in which the molecules are arranged in a random manner so that there is no well-defined arrangement, e.g., molecular packing, and no long-range order.

For example, an amorphous material is a solid material having no sharp characteristic signal(s) in its X-ray power diffractogram (i.e., is not crystalline as determined by XRPD). Instead, one or more broad peaks (e.g., halos) appear in its diffractogram. Broad peaks are characteristic of an amorphous solid. See, e.g., US 2004/0006237 for a comparison of diffractograms of an amorphous material and crystalline material. In addition, the widths of signals in ¹³C NMR, ¹⁹F NMR, and ²³Na NMR spectra of amorphous material are typically substantially broader than those in ¹³C NMR, ¹⁹F NMR, and ²³Na NMR spectra of crystalline material.

As used herein, an X-ray powder diffractogram is “substantially similar to that in [a particular] FIG.” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal maximum values in XRPD diffractograms (in degrees two-theta (° 2θ) referred to herein) generally mean that value reported ±0.2 degrees 2θ of the reported value, an art-recognized variance.

As used herein, an ssNMR spectrum is “substantially similar to that in [a particular] FIG.” when at least 90%, such as at least 95%, at least 98%, or at least 99%, of the signals in the two spectra overlap. In determining “substantial similarity,” one of ordinary skill in the art will understand that there may be variation in the intensities and/or signal positions in ssNMR spectra even for the same crystalline form. Thus, those of ordinary skill in the art will understand that the signal maximum values in ssNMR spectra (in ppm) referred to herein generally mean that value reported ±0.2 ppm of the reported value, an art-recognized variance.

As used herein, a crystalline form is “substantially pure” when it accounts for an amount by weight equal to or greater than 90% of the sum of all solid form(s) in a sample as determined by a method in accordance with the art, such as quantitative XRPD. In some embodiments, the solid form is “substantially pure” when it accounts for an amount by weight equal to or greater than 95% of the sum of all solid form(s) in a sample. In some embodiments, the solid form is “substantially pure” when it accounts for an amount by weight equal to or greater than 99% of the sum of all solid form(s) in a sample.

As used herein, the phrase “substantially amorphous Compound 1” is used interchangeably with the phrases “amorphous Compound 1,” and “amorphous Compound 1 substantially free of crystalline Compound 1.” In some embodiments, substantially amorphous Compound 1 has less than about 30% crystalline Compound 1, for example, less than about 30% of crystalline Compound 1, e.g., less than about 25% crystalline Compound 1, less than about 20% crystalline Compound 1, less than about 15% crystalline Compound 1, less than about 10% crystalline Compound 1, less than about 5% crystalline Compound 1, less than about 2% crystalline Compound 1.

As used herein, the term “DSC” refers to the analytical method of Differential Scanning Calorimetry.

As used herein, the term “TGA” refers to the analytical method of Thermo Gravimetric (or thermogravimetric) Analysis.

As used herein, a “dispersion” refers to a disperse system in which one substance, the dispersed phase, is distributed, in discrete units, throughout a second substance (the continuous phase or vehicle). The size of the dispersed phase can vary considerably (e.g., colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase), or alternatively, an amorphous drug (dispersed phase) in an amorphous polymer (continuous phase). In some embodiments, an amorphous solid dispersion includes the polymer constituting the dispersed phase, and the drug constitutes the continuous phase. In some embodiments, the dispersion includes amorphous Compound 1 or substantially amorphous Compound 1.

The term “solid amorphous dispersion” generally refers to a solid dispersion of two or more components, usually a drug and polymer, but possibly containing other components such as surfactants or other pharmaceutical excipients, where Compound 1 is amorphous or substantially amorphous (e.g., substantially free of crystalline Compound 1), and the physical stability and/or dissolution and/or solubility of the amorphous drug is enhanced by the other components.

The term “tautomer,” as used herein, refers to one of two or more isomers of compound that exist together in equilibrium, and are readily interchanged by migration of an atom, e.g., a hydrogen atom, or group within the molecule.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation, is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom of at least 3500 (52.5% deuterium incorporation at each designated deuterium), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), or at least 6600 (99% deuterium incorporation).

“Selected from” and “chosen from” are used interchangeably herein.

### II. Solid Forms of Compound 1

In some embodiments, the solid form of Compound 1 is an amorphous solid. In some embodiments, the solid form of Compound 1 is a crystalline solid. In some embodiments, the solid form of Compound 1 is Compound 1 neat Form C.

In some embodiments, the solid form of Compound 1 is a salt of Compound 1. In some embodiments, the solid form of Compound 1 is a Na salt of Compound 1. In some embodiments, the solid form of Compound 1 is a Na salt Form A. In some embodiments, the solid form of Compound 1 is a Na salt Form B. In some embodiments, the solid form of Compound 1 is a Na salt Form C. In some embodiments, the solid form of Compound 1 is a Na salt Form D.

In some embodiments, the solid form of Compound 1 is a Ca salt of Compound 1. In some embodiments, the solid form of Compound 1 is a Ca salt Form A.

In some embodiments, the solid form of Compound 1 is a HCl salt of Compound 1. In some embodiments, the solid form of Compound 1 is a HCl salt Form A.

In some embodiments, the solid form of Compound 1 is a solvate of Compound 1. In some embodiments, the solid form of Compound 1 is a DMSO solvate of Compound 1. In some embodiments, the solid form of Compound 1 is a DMSO solvate Form A.

In some embodiments, the solid form of Compound 1 is a EtOH solvate of Compound 1. In some embodiments, the solid form of Compound 1 is a EtOH solvate Form A.

In some embodiments, the solid form of Compound 1 is a salt or cocrystal of Compound 1. In some embodiments, the solid form of Compound 1 is a tartrate salt or cocrystal of Compound 1. In some embodiments, the solid form of Compound 1 is a tartrate salt or cocrystal Form A. In some embodiments, the solid form of Compound 1 is a tartrate salt or cocrystal Form B. In some embodiments, the solid form of Compound 1 is a tartrate salt or cocrystal Form C. In some embodiments, the solid form of Compound 1 is a tartrate salt or cocrystal Form D.

In some embodiments, the solid form of Compound 1 is a solid dispersion comprising a solid form of Compound 1 or a salt, or a solvate, or a cocrystal thereof. In some embodiments, the solid dispersion is a spray-dried dispersion comprising a solid form of Compound 1 or a salt, or a solvate, or a cocrystal thereof. In some embodiments, the solid dispersion comprises one or more polymers. In some embodiments, the one or more polymers in the solid dispersion is/are selected from pyrrolidones, celluloses, poloxamers, polymethacrylate based copolymers, and triblock copolymers. In some embodiments, the solid dispersion comprising amorphous Compound 1 also comprises HPMCAS.

In some embodiments, the solid form of Compound 1 is a mixture of any two or more of the foregoing.

1. Compound 1 Neat Form C

In some embodiments, Compound 1 is a crystalline solid comprising neat crystalline Form C. In some embodiments, the crystalline solid comprises 30% to 99% neat crystalline Compound 1 Form C relative to the total weight of solid Compound 1. In some embodiments, the crystalline solid comprises 40% to 99% neat crystalline Compound 1 Form C relative to the total weight of solid Compound 1. In some embodiments, the crystalline solid comprises 50% to 99% neat crystalline Compound 1 Form C relative to the total weight of solid Compound 1. In some embodiments, the crystalline solid comprises 60% to 99% neat crystalline Compound 1 Form C relative to the total weight of solid Compound 1. In some embodiments, the crystalline solid comprises 70% to 99% neat crystalline Compound 1 Form C relative to the total weight of solid Compound 1. In some embodiments, the crystalline solid comprises 75% to 99% neat crystalline Compound 1 Form C relative to the total weight of solid Compound 1. In some embodiments, the crystalline solid comprises 80% to 99% neat crystalline Compound 1 Form C relative to the total weight of solid Compound 1. In some embodiments, the crystalline solid comprises 85% to 99% neat crystalline Compound 1 Form C relative to the total weight of solid Compound 1. In some embodiments, the crystalline solid comprises 90% to 99% neat crystalline Compound 1 Form C relative to the total weight of solid Compound 1. In some embodiments, the crystalline solid comprises 95% to 99% neat crystalline Compound 1 Form C relative to the total weight of solid Compound 1.

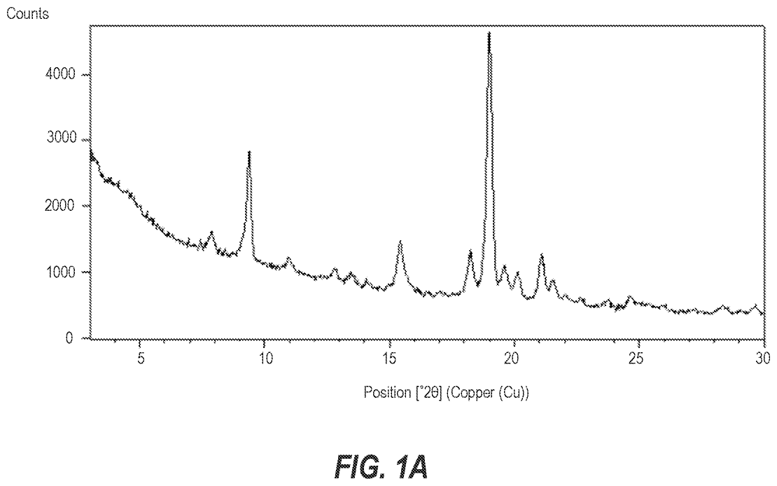

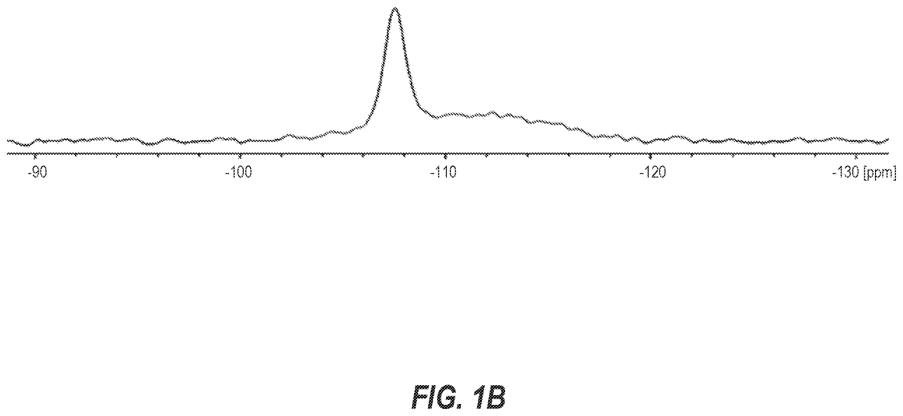

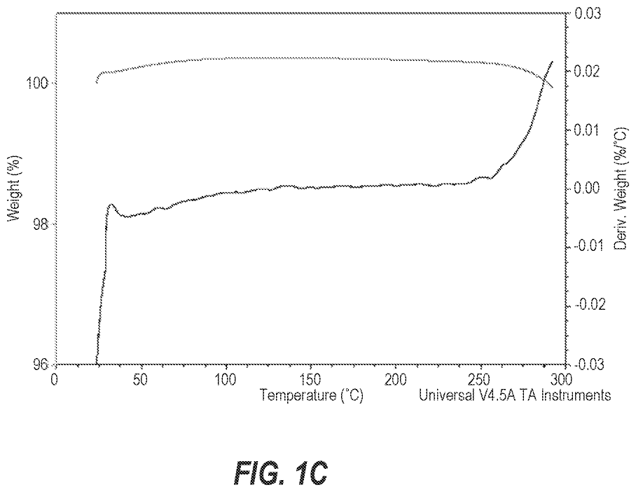

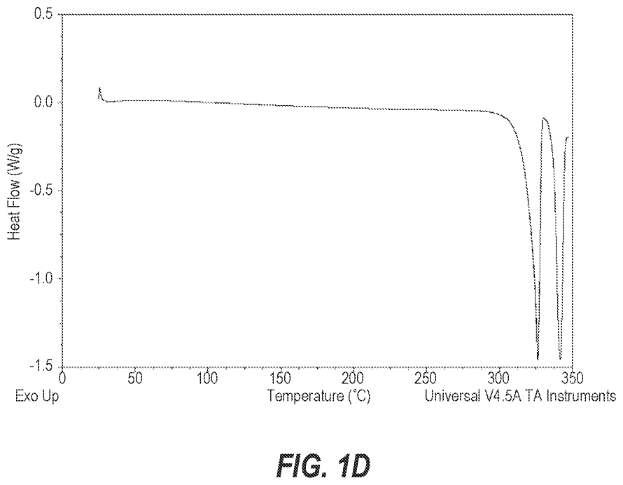

Thus, in some embodiments, Compound 1 neat Form C is substantially crystalline. In some embodiments, Compound 1 neat Form C is substantially pure crystalline. In some embodiments, Compound 1 neat Form C is characterized by an X-ray powder diffractogram generated by an X-ray powder diffraction analysis with an incident beam of Cu Kα radiation. FIG. 1A provides an X-ray powder diffractogram of Compound 1 neat Form C at room temperature.

In some embodiments, Compound 1 neat Form C is characterized by an X-ray powder diffractogram having a signal at 9.4±0.2 degrees two-theta, and a signal at one or more of 15.4±0.2 degrees two-theta, 19.0±0.2 degrees two-theta, and 21.1±0.2 degrees two-theta. In some embodiments, Compound 1 neat Form C is characterized by an X-ray powder diffractogram having a signal at 9.4±0.2 degrees two-theta, and signals at two or more of 15.4±0.2 degrees two-theta, 19.0±0.2 degrees two-theta, and 21.1±0.2 degrees two-theta. In some embodiments, Compound 1 neat Form C is characterized by an X-ray powder diffractogram having signals at 9.4±0.2 degrees two-theta, 19.0±0.2 degrees two-theta, 15.4±0.2 degrees two-theta, and 21.1±0.2 degrees two-theta. In some embodiments, Compound 1 neat Form C is characterized by an X-ray powder diffractogram having (a) signals at 9.4±0.2 degrees two-theta, 15.4±0.2 degrees two-theta, 19.0±0.2 degrees two-theta, and 21.1±0.2 degrees two-theta; and (b) at least one, at least two, or at least three signals selected from 18.2±0.2 degrees two-theta, 19.6±0.2 degrees two-theta, and 20.1±0.2 degrees two-theta.

In some embodiments, Compound 1 neat Form C is characterized by an X-ray powder diffractogram substantially similar to FIG. 1A.

In some embodiments, Compound 1 neat Form C is characterized by a ¹⁹F ssNMR peak at −107.5±0.2 ppm. In some embodiments, Compound 1 neat Form C is characterized by a ¹⁹F ssNMR spectrum substantially similar to FIG. 1B. In some embodiments, Compound 1 neat Form C is characterized by a TGA thermogram substantially similar to FIG. 1C. In some embodiments, Compound 1 neat Form C is characterized by a DSC thermogram substantially similar to FIG. 1D.

Another aspect of the disclosure provides a composition comprising Compound 1 neat Form C. In some embodiments, the composition comprises substantially pure crystalline Compound 1 neat Form C. In some embodiments, the composition consists essentially of Compound 1 neat Form C.

Another aspect of the disclosure provides a method of making Compound 1 neat Form C. In some embodiments, Compound 1 neat Form C is prepared by:
(a) contacting Compound 1 with an organic solvent (e.g., DMSO) to form a first reaction mixture;
(b) heating and stirring the first reaction mixture; and
(c) isolating a solid portion from step (b) and heating the solid portion in an inert environment to yield Compound 1 neat Form C.

2. Compound 1 Na Salt Form A

In some embodiments, Compound 1 Na salt Form A is a crystalline solid comprising crystalline Na salt Form A. In some embodiments, the crystalline solid comprises 30% to 99% crystalline Compound 1 Na salt Form A relative to the total weight of solid Compound 1. In some embodiments, the crystalline solid comprises 40% to 99% crystalline Compound 1 Na salt Form A relative to the total weight of solid Compound 1. In some embodiments, the crystalline solid comprises 50% to 99% crystalline Compound 1 Na salt Form A relative to the total weight of solid Compound 1. In some embodiments, the crystalline solid comprises 60% to 99% crystalline Compound 1 Na salt Form A relative to the total weight of solid Compound 1. In some embodiments, the crystalline solid comprises 70% to 99% crystalline Compound 1 Na salt Form A relative to the total weight of solid Compound 1. In some embodiments, the crystalline solid comprises 75% to 99% crystalline Compound 1 Na salt Form A relative to the total weight of solid Compound 1. In some embodiments, the crystalline solid comprises 80% to 99% crystalline Compound 1 Na salt Form A relative to the total weight of solid Compound 1. In some embodiments, the crystalline solid comprises 85% to 99% crystalline Compound 1 Na salt Form A relative to the total weight of solid Compound 1. In some embodiments, the crystalline solid comprises 90% to 99% crystalline Compound 1 Na salt Form A relative to the total weight of solid Compound 1. In some embodiments, the crystalline solid comprises 95% to 99% crystalline Compound 1 Na salt Form A relative to the total weight of solid Compound 1.

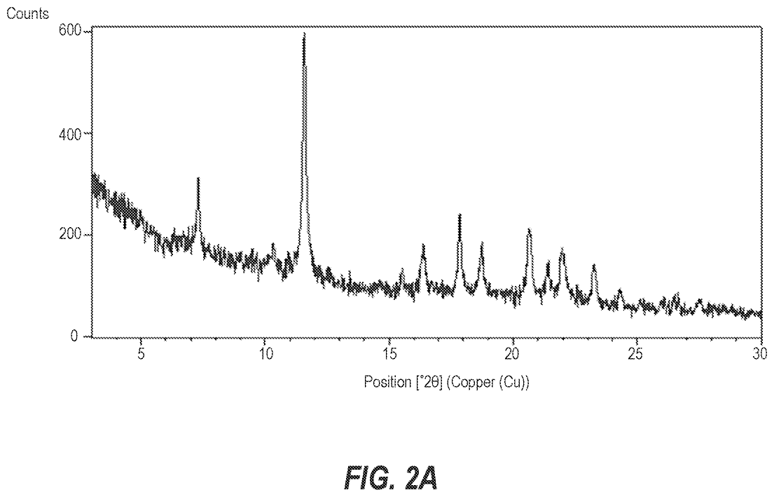

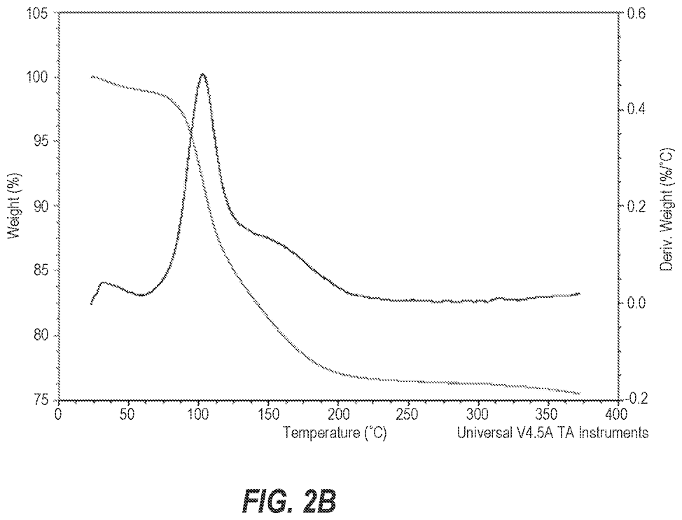

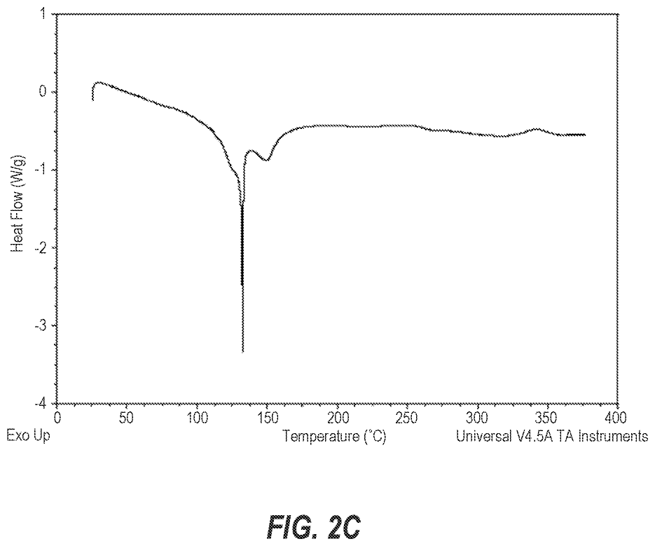

Thus, in some embodiments, Compound 1 Na salt Form A is substantially crystalline. In some embodiments, Compound 1 Na salt Form A is substantially pure crystalline. In some embodiments, Compound 1 Na salt Form A is characterized by an X-ray powder diffractogram generated by an X-ray powder diffraction analysis with an incident beam of Cu Kα radiation. FIG. 2A provides an X-ray powder diffractogram of Compound 1 Na salt Form A at room temperature.

In some embodiments, Compound 1 Na salt Form A is characterized by an X-ray powder diffractogram having a signal at at least one of 7.3±0.2 degrees two-theta and 11.6±0.2 degrees two-theta. In some embodiments, Compound 1 Na salt Form A is characterized by an X-ray powder diffractogram having signals at at least one of 7.3±0.2 degrees two-theta and 11.6±0.2 degrees two-theta, and at least one of 17.8±0.2 degrees two-theta and 20.6±0.2 degrees two-theta. In some embodiments, Compound 1 Na salt Form A is characterized by an X-ray powder diffractogram having signals at 7.3±0.2 degrees two-theta and 11.6±0.2 degrees two-theta, and at least one of 17.8±0.2 degrees two-theta and 20.6±0.2 degrees two-theta. In some embodiments, Compound 1 Na salt Form A is characterized by an X-ray powder diffractogram having signals at 7.3±0.2 degrees two-theta, 11.6±0.2 degrees two-theta, 17.8±0.2 degrees two-theta, and 20.6±0.2 degrees two-theta. In some embodiments, Compound 1 Na Salt Form A is characterized by an X-ray powder diffractogram having (a) signals at 7.3±0.2 degrees two-theta, 11.6±0.2 degrees two-theta, 17.8±0.2 degrees two-theta, and 20.6±0.2 degrees two-theta; and (b) at least one, at least two, or at least three signals selected from 16.4±0.2 degrees two-theta, 23.2±0.2 degrees two-theta, 18.7±0.2 degrees two-theta, 21.4±10.2 degrees two-theta, and 21.9±0.2 degrees two-theta. In some embodiments, Compound 1 Na salt Form A is characterized by an X-ray powder diffractogram substantially similar to FIG. 2A.

In some embodiments, Compound 1 Na salt Form A is characterized by a TGA thermogram substantially similar to FIG. 2B. In some embodiments, Compound 1 Na salt Form A is characterized by a DSC thermogram substantially similar to FIG. 2C.

Another aspect of the disclosure provides a composition comprising Compound 1 Na salt Form A. In some embodiments, the composition comprises substantially pure crystalline Compound 1 Na salt Form A. In some embodiments, the composition consists essentially of Compound 1 Na salt Form A.

Another aspect of the disclosure provides a method of making Compound 1 Na salt Form A. In some embodiments, Compound 1 Na salt Form A is prepared by:
(a) reacting Compound 1 Form A with NaOH in the presence of acetone to form a second reaction mixture; and
(b) isolating a solid portion from step (a) and drying the solid portion to yield Compound 1 Na salt Form A.

In some embodiments, Compound 1 Form A is prepared using the method as described in International Patent Application No. PCT/US2020/032832. In some embodiments, the composition consists essentially of Compound 1 Na salt Form A. Compound 1 Form A is prepared using a method comprising the steps of:
(i) contacting methyl 4-(5-(4-fluorophenyl)-1-pivaloyl-6-(tetrahydro-2H-pyran-4-yl)-1,5-dihydropyrrolo[2,3-f]indazol-7-yl)benzoate with a first organic solvent and a first base to form a first reaction mixture;
(ii) adding water and a first acid to the first reaction mixture;
(iii) isolating an organic portion from step (ii), adding an alcohol and optionally adding water to the organic portion, and concentrating the mixture by distillation; and
(iv) isolating Compound 1 from the mixture from step (iii) and drying the material to remove all water content to yield Compound 1 Form A.

3. Compound 1 Na Salt Form B

In some embodiments, Compound 1 Na salt Form B is a crystalline solid comprising crystalline Na salt Form B. In some embodiments, the crystalline solid comprises 30% to 99% crystalline Compound 1 Na salt Form B relative to the total weight of solid Compound 1. In some embodiments, the crystalline solid comprises 40% to 99% crystalline Compound 1 Na salt Form B relative to the total weight of solid Compound 1. In some embodiments, the crystalline solid comprises 50% to 99% crystalline Compound 1 Na salt Form B relative to the total weight of solid Compound 1. In some embodiments, the crystalline solid comprises 60% to 99% crystalline Compound 1 Na salt Form B relative to the total weight of solid Compound 1. In some embodiments, the crystalline solid comprises 70% to 99% crystalline Compound 1 Na salt Form B relative to the total weight of solid Compound 1. In some embodiments, the crystalline solid comprises 75% to 99% crystalline Compound 1 Na salt Form B relative to the total weight of solid Compound 1. In some embodiments, the crystalline solid comprises 80% to 99% crystalline Compound 1 Na salt Form B relative to the total weight of solid Compound 1. In some embodiments, the crystalline solid comprises 85% to 99% crystalline Compound 1 Na salt Form B relative to the total weight of solid Compound 1. In some embodiments, the crystalline solid comprises 90% to 99% crystalline Compound 1 Na salt Form B relative to the total weight of solid Compound 1. In some embodiments, the crystalline solid comprises 95% to 99% crystalline Compound 1 Na salt Form B relative to the total weight of solid Compound 1.

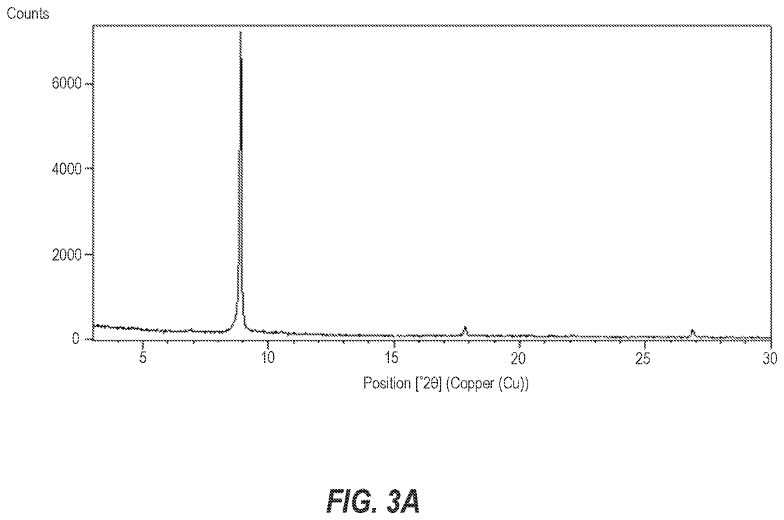

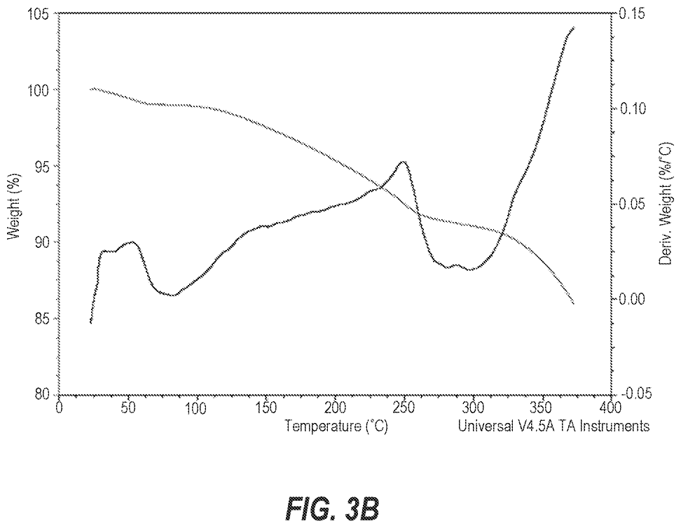

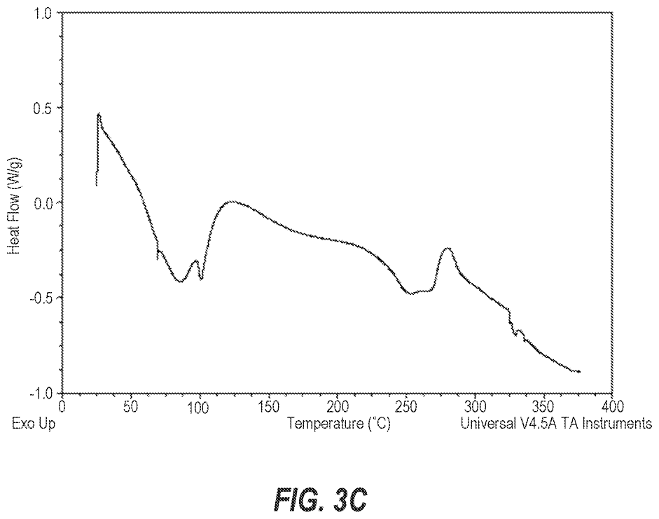

Thus, in some embodiments, Compound 1 Na salt Form B is substantially crystalline. In some embodiments, Compound 1 Na salt Form B is substantially pure crystalline. In some embodiments, Compound 1 Na salt Form B is characterized by an X-ray powder diffractogram generated by an X-ray powder diffraction analysis with an incident beam of Cu Kα radiation. FIG. 3A provides an X-ray powder diffractogram of Compound 1 Na salt Form B at room temperature.

In some embodiments, Compound 1 Na salt Form B is characterized by an X-ray powder diffractogram having signals at 3.1±0.2 degrees two-theta and 8.9±0.2 degrees two-theta. In some embodiments, Compound 1 Na salt Form B is characterized by an X-ray powder diffractogram having signals at 3.1±0.2 degrees two-theta, 8.9±0.2 degrees two-theta, 17.8±0.2 degrees two-theta, and 26.9±0.2 degrees two-theta. In some embodiments, Compound 1 Na salt Form B is characterized by an X-ray powder diffractogram substantially similar to FIG. 3A.

In some embodiments, Compound 1 Na salt Form B is characterized by a TGA thermogram substantially similar to FIG. 3B. In some embodiments, Compound 1 Na salt Form B is characterized by a DSC thermogram substantially similar to FIG. 3C.

Another aspect of the disclosure provides a composition comprising Compound 1 Na salt Form B. In some embodiments, the composition comprises substantially pure crystalline Compound 1 Na salt Form B. In some embodiments, the composition consists essentially of Compound 1 Na salt Form B.

Another aspect of the disclosure provides a method of making Compound 1 Na salt Form B. In some embodiments, Compound 1 Na salt Form B is prepared by:
(a) reacting Compound 1 Form A with NaOH in the presence of ethyl acetate to form a second reaction mixture; and
(b) isolating a solid portion from step (a) and drying the solid portion to yield Compound 1 Na salt Form B.

4. Compound 1 Na Salt Form C

In some embodiments, Compound 1 Na salt Form C is a crystalline solid comprising crystalline Na salt Form C. In some embodiments, the crystalline solid comprises 30% to 99% crystalline Compound 1 Na salt Form C relative to the total weight of solid Compound 1. In some embodiments, the crystalline solid comprises 40% to 99% crystalline Compound 1 Na salt Form C relative to the total weight of solid Compound 1. In some embodiments, the crystalline solid comprises 50% to 99% crystalline Compound 1 Na salt Form C relative to the total weight of solid Compound 1. In some embodiments, the crystalline solid comprises 60% to 99% crystalline Compound 1 Na salt Form C relative to the total weight of solid Compound 1. In some embodiments, the crystalline solid comprises 70% to 99% crystalline Compound 1 Na salt Form C relative to the total weight of solid Compound 1. In some embodiments, the crystalline solid comprises 75% to 99% crystalline Compound 1 Na salt Form C relative to the total weight of solid Compound 1. In some embodiments, the crystalline solid comprises 80% to 99% crystalline Compound 1 Na salt Form C relative to the total weight of solid Compound 1. In some embodiments, the crystalline solid comprises 85% to 99% crystalline Compound 1 Na salt Form C relative to the total weight of solid Compound 1. In some embodiments, the crystalline solid comprises 90% to 99% crystalline Compound 1 Na salt Form C relative to the total weight of solid Compound 1. In some embodiments, the crystalline solid comprises 95% to 99% crystalline Compound 1 Na salt Form C relative to the total weight of solid Compound 1.

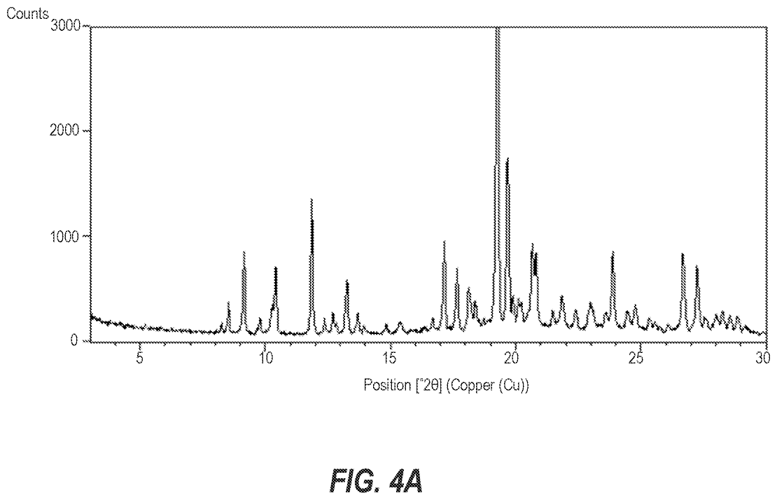

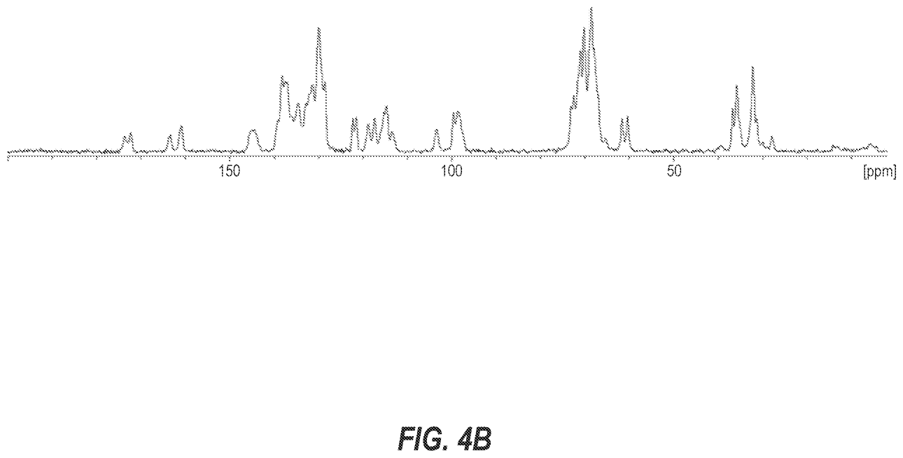

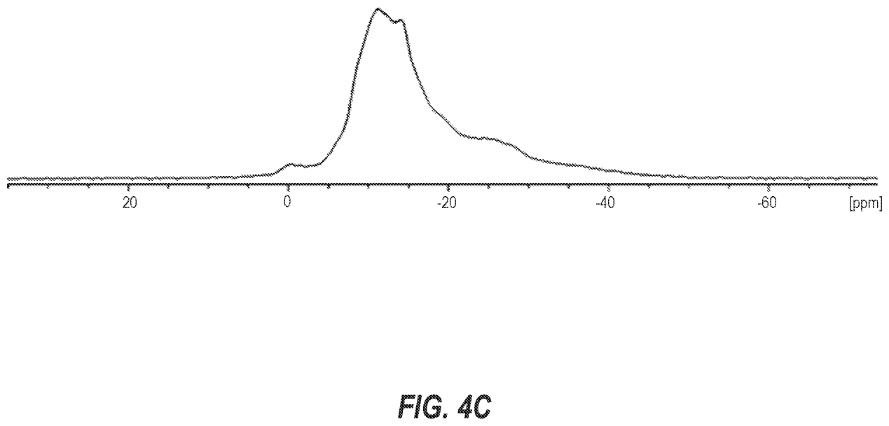

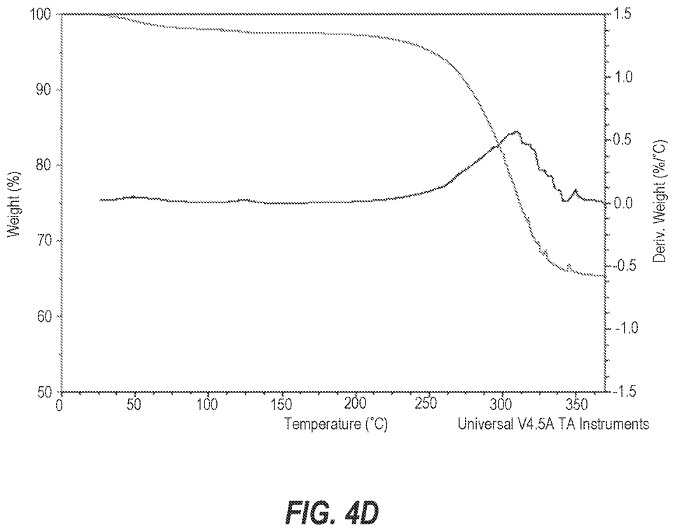

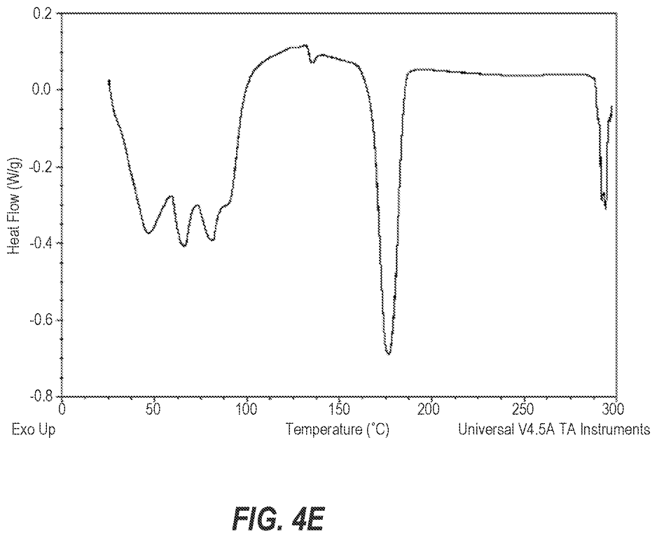

Thus, in some embodiments, Compound 1 Na salt Form C is substantially crystalline. In some embodiments, Compound 1 Na salt Form C is substantially pure crystalline. In some embodiments, Compound 1 Na salt Form C is characterized by an X-ray powder diffractogram generated by an X-ray powder diffraction analysis with an incident beam of Cu Kα radiation. FIG. 4A provides an X-ray powder diffractogram of Compound 1 Na salt Form C at room temperature.

In some embodiments, Compound 1 Na salt Form C is characterized by an X-ray powder diffractogram having signals at 19.7±0.2 degrees two-theta, 9.2±0.2 degrees two-theta, and 13.3±0.2 degrees two-theta. In some embodiments, Compound 1 Na salt Form C is characterized by an X-ray powder diffractogram having (a) signals at 19.7±0.2 degrees two-theta, 9.2±0.2 degrees two-theta, and 13.3±0.2 degrees two-theta; and (b) at least one, at least two, or at least three signals selected from 10.4±0.2 degrees two-theta, 11.9±0.2 degrees two-theta, 17.1±0.2 degrees two-theta, 17.7±0.2 degrees two-theta, 20.7±0.2 degrees two-theta, 19.2±0.2 degrees two-theta, 20.8±0.2 degrees two-theta, 23.9±0.2 degrees two-theta, 26.6±0.2 degrees two-theta, 26.7±0.2 degrees two-theta, and 27.2±0.2 degrees two-theta.

In some embodiments, Compound 1 Na salt Form C is characterized by an X-ray powder diffractogram substantially similar to FIG. 4A.

In some embodiments, Compound 1 Na salt Form C is characterized by having a ¹³C ssNMR peak at one or more of 138.1±0.2 ppm, 121.5±0.2 ppm, 117.4±0.2 ppm, 115.2±0.2 ppm, 36.7±0.2 ppm, and 32.1±0.2 ppm. In some embodiments, Compound 1 Na Salt Form C is characterized by having a ¹³C ssNMR peak at two, three, or four of 138.1±0.2 ppm, 121.5±0.2 ppm, 117.4±0.2 ppm, 115.2±0.2 ppm, 36.7±0.2 ppm, and 32.1±0.2 ppm. In some embodiments, Compound 1 Na salt Form C is characterized by having a ¹³C ssNMR peak at 138.1±0.2 ppm, 121.5±0.2 ppm, 117.4±0.2 ppm, 115.2±0.2 ppm, 36.7±0.2 ppm, and 32.1±0.2 ppm. In some embodiments, Compound 1 Na salt Form C is characterized by having (a) a ¹³C ssNMR peak at 138.1±0.2 ppm, 121.5±0.2 ppm, 117.4±0.2 ppm, 115.2±0.2 ppm, 36.7±0.2 ppm, and 32.1±0.2 ppm; and (b) a ¹³C ssNMR peak at one, two, three, four, or more of 173.7±0.2 ppm, 172.3±0.2 ppm, 145.0±0.2 ppm, 144.5±0.2 ppm, 103.4±0.2 ppm, 99.6±0.2 ppm, 72.4±0.2 ppm, 70.9±0.2 ppm, 70.2±0.2 ppm, 68.5±0.2 ppm, 61.6±0.2 ppm, 60.3±0.2 ppm, and 31.3±0.2 ppm. In some embodiments, Compound 1 Na salt Form C is characterized by a ¹³C ssNMR spectrum substantially similar to FIG. 4B.

In some embodiments, Compound 1 Na salt Form C is characterized by a ²³Na ssNMR peak at −11.2±0.2 ppm and/or −14.0±0.2 ppm. In some embodiments, Compound 1 Na salt Form C is characterized by a ²³Na ssNMR spectrum substantially similar to FIG. 4C.

In some embodiments, Compound 1 Na salt Form C is characterized by a TGA thermogram substantially similar to FIG. 4D. In some embodiments, Compound 1 Na salt Form C is characterized by a DSC thermogram substantially similar to FIG. 4E.

Another aspect of the disclosure provides a composition comprising Compound 1 Na salt Form C. In some embodiments, the composition comprises substantially pure crystalline Compound 1 Na salt Form C. In some embodiments, the composition consists essentially of Compound 1 Na salt Form C.

Another aspect of the disclosure provides a method of making Compound 1 Na salt Form C. In some embodiments, Compound 1 Na salt Form C is prepared by:
(a) reacting Compound 1 Form A with NaOH in the presence of a polyethylene glycol (e.g., tocopherol polyethylene glycol aqueous solution or TPGS) to form a second reaction mixture; and
(b) isolating a solid portion from step (a) to yield Compound 1 Na salt Form C.

5. Compound 1 Na Salt Form D

In some embodiments, Compound 1 Na salt Form D is a crystalline solid comprising crystalline Na salt Form D. In some embodiments, the crystalline solid comprises 30% to 99% crystalline Compound 1 Na salt Form D relative to the total weight of solid Compound 1. In some embodiments, the crystalline solid comprises 40% to 99% crystalline Compound 1 Na salt Form D relative to the total weight of solid Compound 1. In some embodiments, the crystalline solid comprises 50% to 99% crystalline Compound 1 Na salt Form D relative to the total weight of solid Compound 1. In some embodiments, the crystalline solid comprises 60% to 99% crystalline Compound 1 Na salt Form D relative to the total weight of solid Compound 1. In some embodiments, the crystalline solid comprises 70% to 99% crystalline Compound 1 Na salt Form D relative to the total weight of solid Compound 1. In some embodiments, the crystalline solid comprises 75% to 99% crystalline Compound 1 Na salt Form D relative to the total weight of solid Compound 1. In some embodiments, the crystalline solid comprises 80% to 99% crystalline Compound 1 Na salt Form D relative to the total weight of solid Compound 1. In some embodiments, the crystalline solid comprises 85% to 99% crystalline Compound 1 Na salt Form D relative to the total weight of solid Compound 1. In some embodiments, the crystalline solid comprises 90% to 99% h crystalline Compound 1 Na salt Form D relative to the total weight of solid Compound 1. In some embodiments, the crystalline solid comprises 95% to 99% crystalline Compound 1 Na salt Form D relative to the total weight of solid Compound 1.

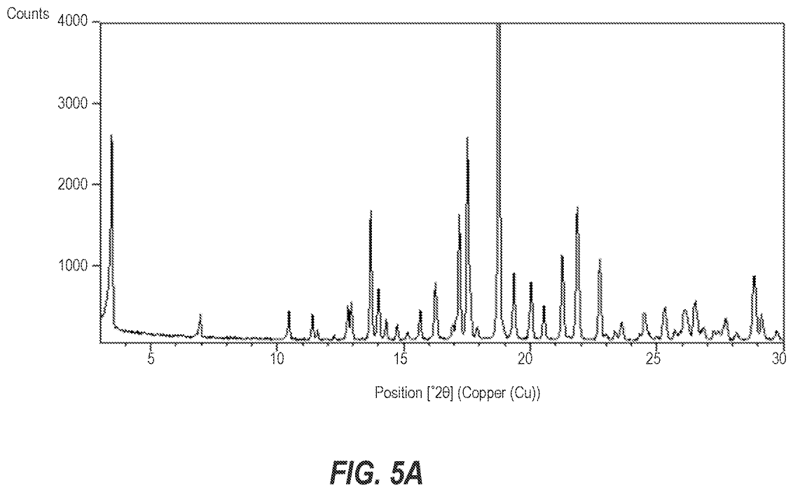

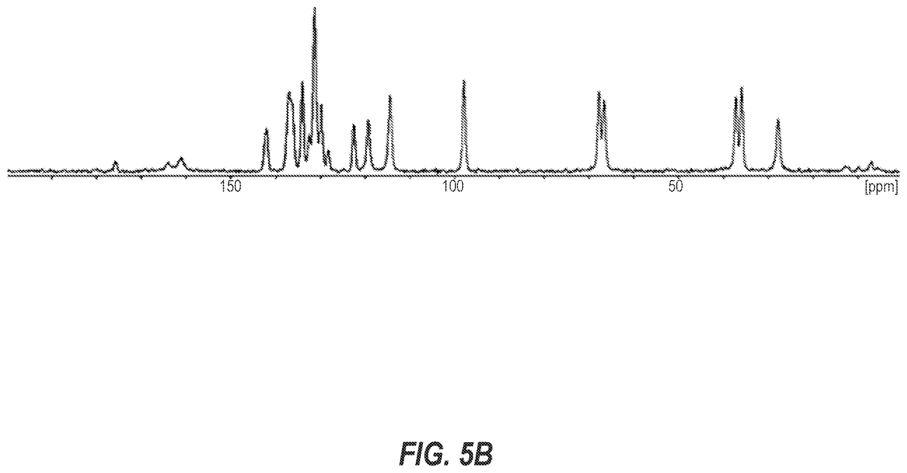

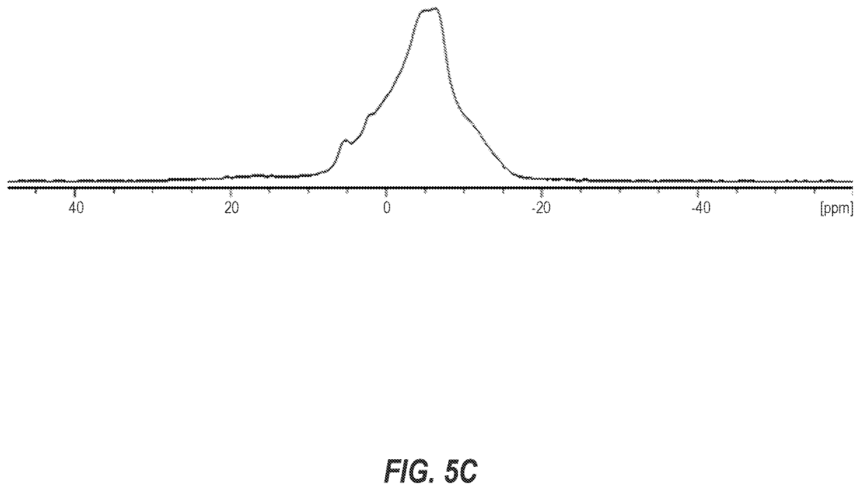

Thus, in some embodiments, Compound 1 Na salt Form D is substantially crystalline. In some embodiments, Compound 1 Na salt Form D is substantially pure crystalline. In some embodiments, Compound 1 Na salt Form D is characterized by an X-ray powder diffractogram generated by an X-ray powder diffraction analysis with an incident beam of Cu Kα radiation. FIG. 5A provides an X-ray powder diffractogram of Compound 1 Na salt Form D at room temperature.

In some embodiments, Compound 1 Na salt Form D is characterized by an X-ray powder diffractogram having signals at 3.5-10.2 degrees two-theta and 16.2±0.2 degrees two-theta. In some embodiments, Compound 1 Na salt Form D is characterized by an X-ray powder diffractogram having signals at 3.5±0.2 degrees two-theta and 16.2±0.2 degrees two-theta and at least one of 18.7±0.2 degrees two-theta, and 17.5±0.2 degrees two-theta. In some embodiments, Compound 1 Na salt Form D is characterized by an X-ray powder diffractogram having signals at 3.5±0.2 degrees two-theta, 16.2±0.2 degrees two-theta, 18.7±0.2 degrees two-theta, and 17.5±0.2 degrees two-theta. In some embodiments, Compound 1 Na Salt Form D is characterized by an X-ray powder diffractogram having (a) signals at 3.5±0.2 degrees two-theta, 16.2±0.2 degrees two-theta, 18.7±0.2 degrees two-theta, and 17.5±0.2 degrees two-theta; and (b) at least one, at least two, or at least three signals selected from 13.7±0.2 degrees two-theta, 14.0±0.2 degrees two-theta, 17.2±0.2 degrees two-theta, 19.3±0.2 degrees two-theta, 20.0±0.2 degrees two-theta, 21.3±0.2 degrees two-theta, 21.8±0.2 degrees two-theta, 22.7±0.2 degrees two-theta, 28.8±0.2 degrees two-theta, and 30.9±0.2 degrees two-theta.

In some embodiments, Compound 1 Na salt Form D is characterized by an X-ray powder diffractogram substantially similar to FIG. 5A.

In some embodiments, Compound 1 Na salt Form C is characterized by having a ¹³C ssNMR peak at one or more of 175.8±0.2 ppm, 142.0±0.2 ppm, 134.0±0.2 ppm, 119.3±0.2 ppm, 97.9±0.2 ppm, 67.7±0.2 ppm, and 37.2±0.2 ppm. In some embodiments, Compound 1 Na salt Form C is characterized by having a ¹³C ssNMR peak at two, three, four, or more of 175.8±0.2 ppm, 142.0±0.2 ppm, 134.0±0.2 ppm, 119.3±0.2 ppm, 97.9±0.2 ppm, 67.7±0.2 ppm, and 37.2±0.2 ppm. In some embodiments, Compound 1 Na salt Form C is characterized by having a ¹³C ssNMR peak at 175.8±0.2 ppm, 142.0±0.2 ppm, 134.0±0.2 ppm, 119.3±0.2 ppm, 97.9±0.2 ppm, 67.7±0.2 ppm, and 37.2±0.2 ppm. In some embodiments, Compound 1 Na salt Form D is characterized by a ¹³C ssNMR spectrum substantially similar to FIG. 5B.

In some embodiments, Compound 1 Na salt Form D is characterized by having a ²³Na ssNMR peak at one or more of 5.3±0.2 ppm, 2.1±0.2 ppm, −5.0±0.2 ppm, and −6.3±0.2 ppm. In some embodiments, Compound 1 Na salt Form D is characterized by having a ²³Na ssNMR peak at two or more of 5.3±0.2 ppm, 2.1±0.2 ppm, −5.0±0.2 ppm, and −6.3±0.2 ppm. In some embodiments, Compound 1 Na salt Form D is characterized by having a ²³Na ssNMR peak at 5.3±0.2 ppm, 2.1±0.2 ppm, −5.0±0.2 ppm, and −6.3±0.2 ppm. In some embodiments, Compound 1 Na salt Form D is characterized by a ²³Na ssNMR spectrum substantially similar to FIG. 5C.

Another aspect of the disclosure provides a composition comprising Compound 1 Na salt Form D. In some embodiments, the composition comprises substantially pure crystalline Compound 1 Na salt Form D. In some embodiments, the composition consists essentially of Compound 1 Na salt Form D.

Another aspect of the disclosure provides a method of making Compound 1 Na salt Form D. In some embodiments, Compound 1 Na salt Form D is prepared by:
(a) reacting Compound 1 Form A with NaOH at 4-10° C. (e.g., 5° C.) to form a second reaction mixture; and
(b) isolating a solid portion from step (a) to yield Compound 1 Na salt Form D.

6. Compound 1 Ca Salt Form A

In some embodiments, Compound 1 Ca salt Form A is a crystalline solid comprising crystalline Ca salt Form A. In some embodiments, the crystalline solid comprises 30% to 99% crystalline Compound 1 Ca salt Form A relative to the total weight of solid Compound 1. In some embodiments, the crystalline solid comprises 40% to 99% crystalline Compound 1 Ca salt Form A relative to the total weight of solid Compound 1. In some embodiments, the crystalline solid comprises 50% to 99% crystalline Compound 1 Ca salt Form A relative to the total weight of solid Compound 1. In some embodiments, the crystalline solid comprises 60% to 99% crystalline Compound 1 Ca salt Form A relative to the total weight of solid Compound 1. In some embodiments, the crystalline solid comprises 70% to 99% crystalline Compound 1 Ca salt Form A relative to the total weight of solid Compound 1. In some embodiments, the crystalline solid comprises 75% to 99% crystalline Compound 1 Ca salt Form A relative to the total weight of solid Compound 1. In some embodiments, the crystalline solid comprises 80% to 99% crystalline Compound 1 Ca salt Form A relative to the total weight of solid Compound 1. In some embodiments, the crystalline solid comprises 85% to 99% crystalline Compound 1 Ca salt Form A relative to the total weight of solid Compound 1. In some embodiments, the crystalline solid comprises 90% to 99% crystalline Compound 1 Ca salt Form A relative to the total weight of solid Compound 1. In some embodiments, the crystalline solid comprises 95% to 99% crystalline Compound 1 Ca salt Form A relative to the total weight of solid Compound 1.

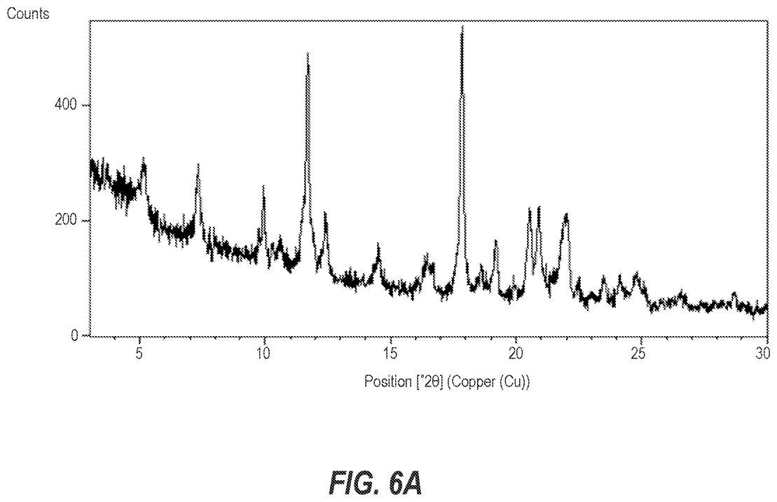

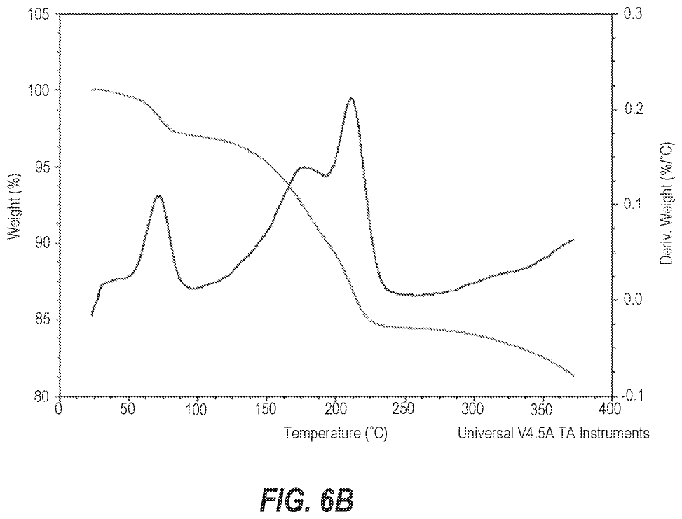

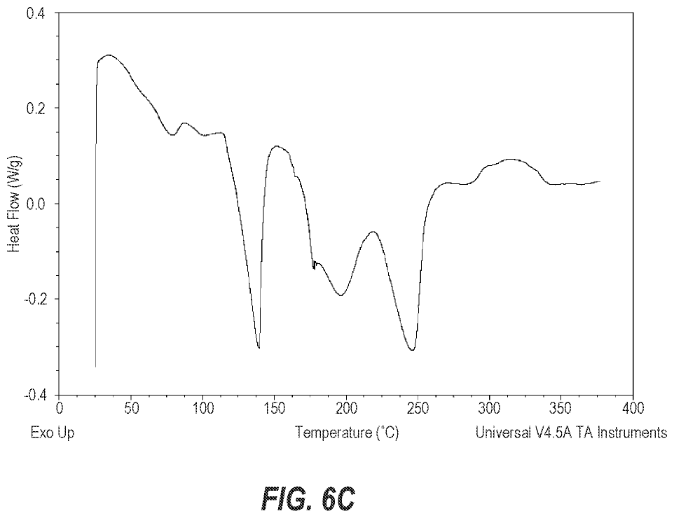

Thus, in some embodiments, Compound 1 Ca salt Form A is substantially crystalline. In some embodiments, Compound 1 Ca salt Form A is substantially pure crystalline. In some embodiments, Compound 1 Ca salt Form A is characterized by an X-ray powder diffractogram generated by an X-ray powder diffraction analysis with an incident beam of Cu Kα radiation. FIG. 6A provides an X-ray powder diffractogram of Compound 1 Ca salt Form A at room temperature.

In some embodiments, Compound 1 Ca salt Form A is characterized by an X-ray powder diffractogram having signals at 17.9±0.2 degrees two-theta and at least one of 11.7±0.2 degrees two-theta and 20.5±0.2 degrees two-theta. In some embodiments, Compound 1 Ca salt Form A is characterized by an X-ray powder diffractogram having signals at 17.9±0.2 degrees two-theta, 11.7±0.2 degrees two-theta, and 20.5±0.2 degrees two-theta. In some embodiments, Compound 1 Ca salt Form A is characterized by an X-ray powder diffractogram having (a) signals at 17.9±0.2 degrees two-theta, 11.7±0.2 degrees two-theta, and 20.5±0.2 degrees two-theta; and (b) at least one, at least two, or at least three signals selected from 5.2±0.2 degrees two-theta, 7.3±0.2 degrees two-theta, 9.9±0.2 degrees two-theta, 10.6±0.2 degrees two-theta, 12.4±0.2 degrees two-theta, 14.5±0.2 degrees two-theta, 16.4±0.2 degrees two-theta, 18.6±0.2 degrees two-theta, 19.2±0.2 degrees two-theta, 20.9±0.2 degrees two-theta, 22.0±0.2 degrees two-theta, 23.5±0.2 degrees two-theta, 24.1±0.2 degrees two-theta, and 24.7±0.2 degrees two-theta.

In some embodiments, Compound 1 Ca salt Form A is characterized by an X-ray powder diffractogram substantially similar to FIG. 6A.

In some embodiments, Compound 1 Ca salt Form A is characterized by a TGA thermogram substantially similar to FIG. 6B. In some embodiments, Compound 1 Ca salt Form A is characterized by a DSC thermogram substantially similar to FIG. 6C.

Another aspect of the disclosure provides a composition comprising Compound 1 Ca salt Form A. In some embodiments, the composition comprises substantially pure crystalline Compound 1 Ca salt Form A. In some embodiments, the composition consists essentially of Compound 1 Ca salt Form A.

Another aspect of the disclosure provides a method of making Compound 1 Ca salt Form A. In some embodiments, Compound 1 Ca salt Form A is prepared by:
(a) reacting Compound 1 Form A with Ca(OH)₂ in the presence of a second organic solvent (e.g., THF or THF/water mixture) to form a second reaction mixture; and
(b) isolating a solid portion from step (a) and drying the solid portion to yield Compound 1 Ca salt Form A.

7. Compound 1 MCI Salt Form A

In some embodiments, Compound 1 HCl salt Form A is a crystalline solid comprising crystalline HCl salt Form A. In some embodiments, the crystalline solid comprises 30% to 99% crystalline Compound 1 HCl salt Form A relative to the total weight of solid Compound 1. In some embodiments, the crystalline solid comprises 40% to 99% crystalline Compound 1 HCl salt Form A relative to the total weight of solid Compound 1. In some embodiments, the crystalline solid comprises 50% to 99% crystalline Compound 1 HCl salt Form A relative to the total weight of solid Compound 1. In some embodiments, the crystalline solid comprises 60% to 99% crystalline Compound 1 HCl salt Form A relative to the total weight of solid Compound 1. In some embodiments, the crystalline solid comprises 70% to 99% crystalline Compound 1 HCl salt Form A relative to the total weight of solid Compound 1. In some embodiments, the crystalline solid comprises 75% to 99% crystalline Compound 1 HCl salt Form A relative to the total weight of solid Compound 1. In some embodiments, the crystalline solid comprises 80% to 99% crystalline Compound 1 HCl salt Form A relative to the total weight of solid Compound 1. In some embodiments, the crystalline solid comprises 85% to 99% crystalline Compound 1 HCl salt Form A relative to the total weight of solid Compound 1. In some embodiments, the crystalline solid comprises 90% to 99% crystalline Compound 1 HCl salt Form A relative to the total weight of solid Compound 1. In some embodiments, the crystalline solid comprises 95% to 99% crystalline Compound 1 HCl salt Form A relative to the total weight of solid Compound 1.

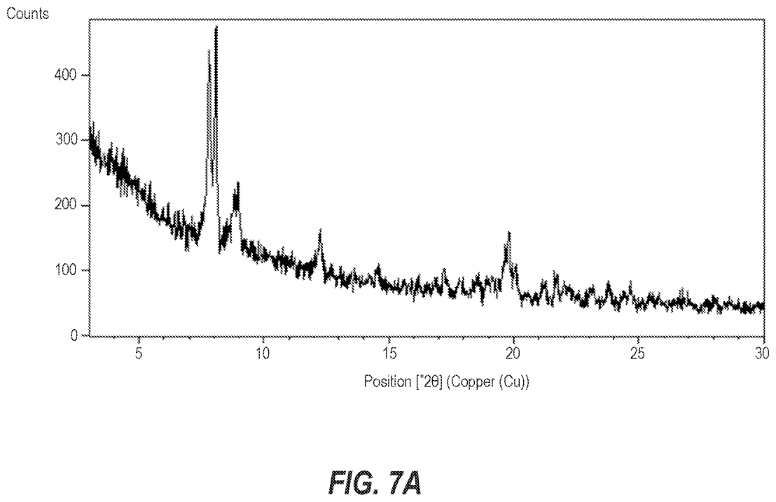

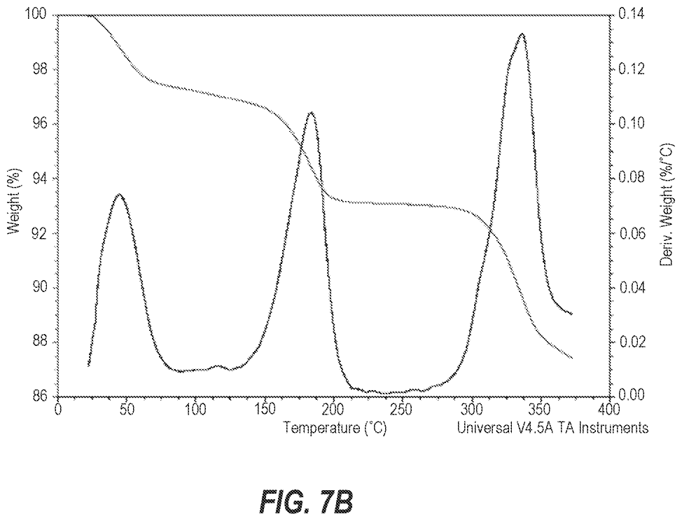

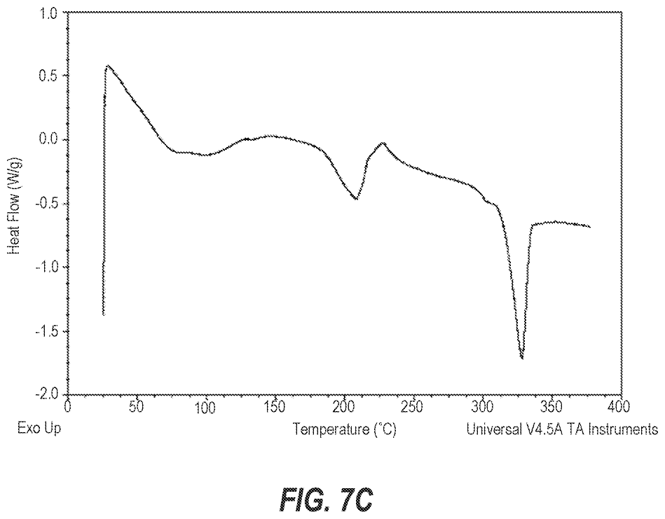

Thus, in some embodiments, Compound 1 HCl salt Form A is substantially crystalline. In some embodiments, Compound 1 HCl salt Form A is substantially pure crystalline. In some embodiments, Compound 1 HCl salt Form A is characterized by an X-ray powder diffractogram generated by an X-ray powder diffraction analysis with an incident beam of Cu Kα radiation. FIG. 7A provides an X-ray powder diffractogram of Compound 1 HCl salt Form A at room temperature.

In some embodiments, Compound 1 HCl salt Form A is characterized by an X-ray powder diffractogram having a signal at one or more of 8.1±0.2 degrees two-theta, 7.8±0.2 degrees two-theta, and 9.0±0.2 degrees two-theta. In some embodiments, Compound 1 HCl salt Form A is characterized by an X-ray powder diffractogram having signals at 8.1±0.2 degrees two-theta, 7.8±0.2 degrees two-theta, and 9.0±0.2 degrees two-theta. In some embodiments, Compound 1 HCl salt Form A is characterized by an X-ray powder diffractogram having (a) signals at 8.1±0.2 degrees two-theta, 7.8±0.2 degrees two-theta, and 9.0±0.2 degrees two-theta; and (b) at least one, at least two, or at least three signals selected from 19.8±0.2 degrees two-theta, 20.1±0.2 degrees two-theta, and 23.8±0.2 degrees two-theta.

In some embodiments, Compound 1 HCl salt Form A is characterized by an X-ray powder diffractogram substantially similar to FIG. 7A.

In some embodiments, Compound 1 HCl salt Form A is characterized by a TGA thermogram substantially similar to FIG. 7B. In some embodiments, Compound 1 HCl salt Form A is characterized by a DSC thermogram substantially similar to FIG. 7C.

Another aspect of the disclosure provides a composition comprising Compound 1 HCl salt Form A. In some embodiments, the composition comprises substantially pure crystalline Compound 1 HCl salt Form A. In some embodiments, the composition consists essentially of Compound 1 HCl salt Form A.

Another aspect of the disclosure provides a method of making Compound 1 HCl salt Form A. In some embodiments, Compound 1 HCl salt Form A is prepared by:
(a) reacting Compound 1 Form A with HCl in the presence of a second organic solvent (e.g., acetonitrile) via slurry to form a second reaction mixture; and
(b) isolating a solid portion from step (a) and drying the solid portion to yield Compound 1 HCl salt Form A.

8. Compound 1 DMSO Solvate Form A

In some embodiments, Compound 1 DMSO solvate Form A is a crystalline solid comprising crystalline DMSO solvate Form A. In some embodiments, the crystalline solid comprises 30% to 99% crystalline Compound 1 DMSO solvate Form A relative to the total weight of solid Compound 1. In some embodiments, the crystalline solid comprises 40% to 99% crystalline Compound 1 DMSO solvate Form A relative to the total weight of solid Compound 1. In some embodiments, the crystalline solid comprises 50% to 99% crystalline Compound 1 DMSO solvate Form A relative to the total weight of solid Compound 1. In some embodiments, the crystalline solid comprises 60% to 99% crystalline Compound 1 DMSO solvate Form A relative to the total weight of solid Compound 1. In some embodiments, the crystalline solid comprises 70% to 99% crystalline Compound 1 DMSO solvate Form A relative to the total weight of solid Compound 1. In some embodiments, the crystalline solid comprises 75% to 990 crystalline Compound 1 DMSO solvate Form A relative to the total weight of solid Compound 1. In some embodiments, the crystalline solid comprises 80% to 99% crystalline Compound 1 DMSO solvate Form A relative to the total weight of solid Compound 1. In some embodiments, the crystalline solid comprises 85% to 99% crystalline Compound 1 DMSO solvate Form A relative to the total weight of solid Compound 1. In some embodiments, the crystalline solid comprises 90% to 99% crystalline Compound 1 DMSO solvate Form A relative to the total weight of solid Compound 1. In some embodiments, the crystalline solid comprises 95% to 99% crystalline Compound 1 DMSO solvate Form A relative to the total weight of solid Compound 1.

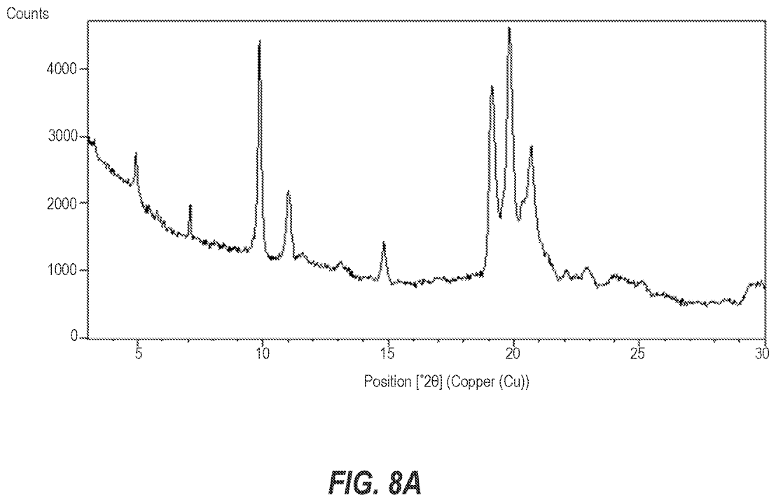

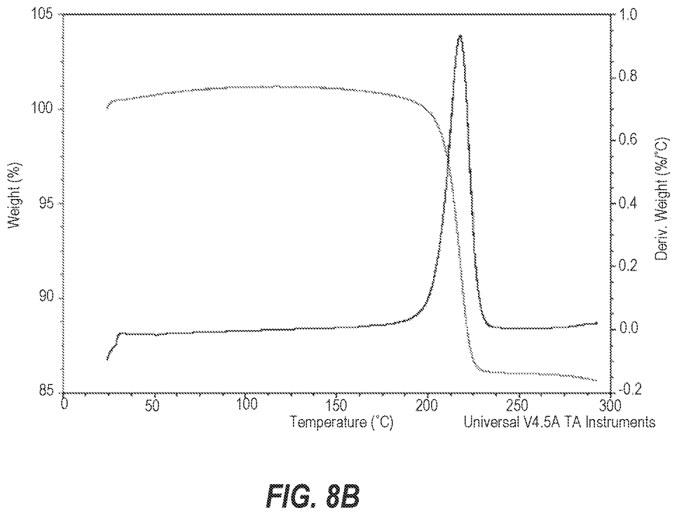

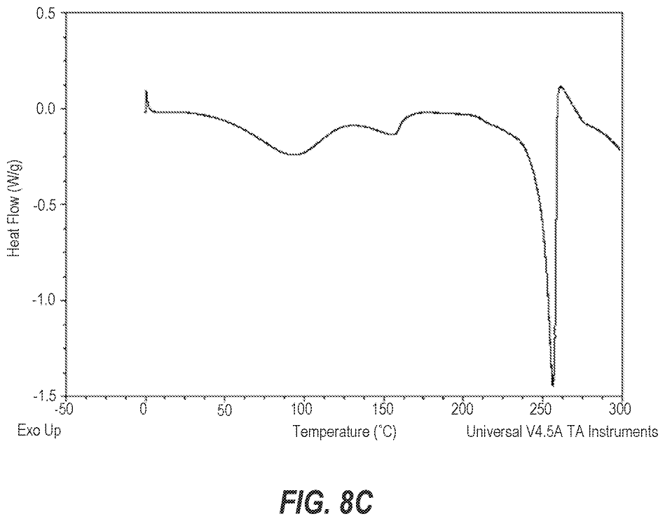

Thus, in some embodiments, Compound 1 DMSO solvate Form A is substantially crystalline. In some embodiments, Compound 1 DMSO solvate Form A is substantially pure crystalline. In some embodiments, Compound 1 DMSO solvate Form A is characterized by an X-ray powder diffractogram generated by an X-ray powder diffraction analysis with an incident beam of Cu Kα radiation. FIG. 8A provides an X-ray powder diffractogram of Compound 1 DMSO solvate Form A at room temperature.

In some embodiments, Compound 1 DMSO solvate Form A is characterized by an X-ray powder diffractogram having a signal at one or more of 9.9±0.2 degrees two-theta, 19.1±0.2 degrees two-theta, and 19.8±0.2 degrees two-theta. In some embodiments, Compound 1 DMSO solvate Form A is characterized by an X-ray powder diffractogram having signals at 9.9±0.2 degrees two-theta, 19.1±0.2 degrees two-theta, and 19.8±0.2 degrees two-theta. In some embodiments, Compound 1 DMSO solvate Form A is characterized by an X-ray powder diffractogram having (a) signals at 9.9±0.2 degrees two-theta, 19.1±0.2 degrees two-theta, and 19.8±0.2 degrees two-theta; and (b) at least one, at least two, or at least three signals selected from 4.9±0.2 degrees two-theta, 7.1±0.2 degrees two-theta, 11.0±0.2 degrees two-theta, 14.8±0.2 degrees two-theta, and 20.7±0.2 degrees two-theta. In some embodiments, Compound 1 DMSO solvate Form A is characterized by an X-ray powder diffractogram substantially similar to FIG. 8A.

In some embodiments, Compound 1 DMSO solvate Form A is characterized by a TGA thermogram substantially similar to FIG. 8B. In some embodiments, Compound 1 DMSO solvate Form A is characterized by a DSC thermogram substantially similar to FIG. 8C.

Another aspect of the disclosure provides a composition comprising Compound 1 DMSO solvate Form A. In some embodiments, the composition comprises substantially pure crystalline Compound 1 DMSO solvate Form A. In some embodiments, the composition consists essentially of Compound 1 DMSO solvate Form A.

Another aspect of the disclosure provides a method of making Compound 1 DMSO solvate Form A. In some embodiments, Compound 1 DMSO solvate Form A is prepared by:
(a) reacting Compound 1 Form A with DMSO to form a second reaction mixture at 90-110° C. (e.g., 100° C.); and
(b) isolating a solid portion from step (a) and drying the solid portion to yield Compound 1 DMSO solvate Form A.

9. Compound 1 EtOH Solvate Form A

In some embodiments, Compound 1 EtOH solvate Form A is a crystalline solid comprising crystalline EtOH solvate Form A. In some embodiments, the crystalline solid comprises 30% to 99% crystalline Compound 1 EtOH solvate Form A relative to the total weight of solid Compound 1. In some embodiments, the crystalline solid comprises 40% to 99% crystalline Compound 1 EtOH solvate Form A relative to the total weight of solid Compound 1. In some embodiments, the crystalline solid comprises 50% to 99% crystalline Compound 1 EtOH solvate Form A relative to the total weight of solid Compound 1. In some embodiments, the crystalline solid comprises 60% to 99% crystalline Compound 1 EtOH solvate Form A relative to the total weight of solid Compound 1. In some embodiments, the crystalline solid comprises 70% to 99% crystalline Compound 1 EtOH solvate Form A relative to the total weight of solid Compound 1. In some embodiments, the crystalline solid comprises 75% to 99% crystalline Compound 1 EtOH solvate Form A relative to the total weight of solid Compound 1. In some embodiments, the crystalline solid comprises 80% to 99% crystalline Compound 1 EtOH solvate Form A relative to the total weight of solid Compound 1. In some embodiments, the crystalline solid comprises 85% to 99% crystalline Compound 1 EtOH solvate Form A relative to the total weight of solid Compound 1. In some embodiments, the crystalline solid comprises 90% to 99% crystalline Compound 1 EtOH solvate Form A relative to the total weight of solid Compound 1. In some embodiments, the crystalline solid comprises 95% to 99% crystalline Compound 1 EtOH solvate Form A relative to the total weight of solid Compound 1.

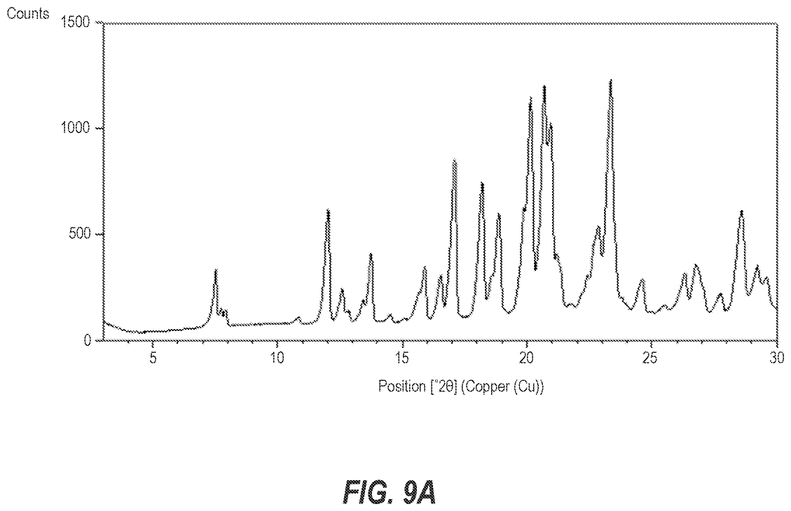

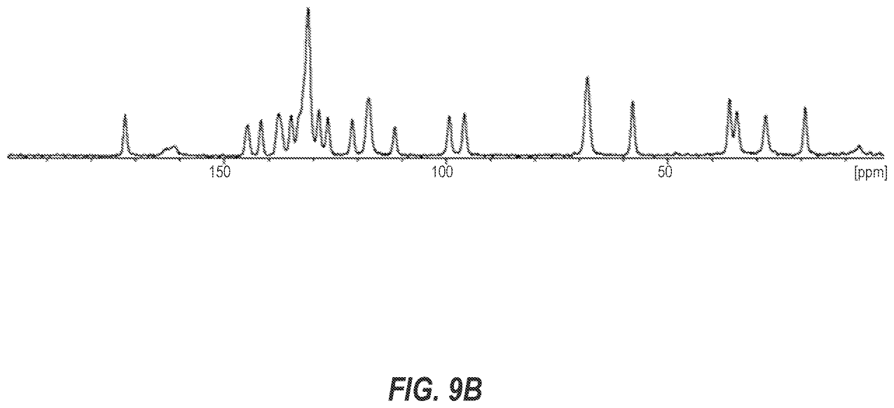

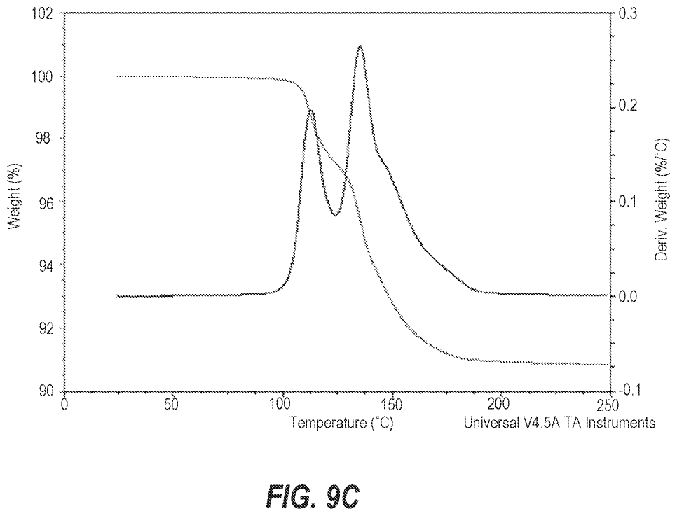

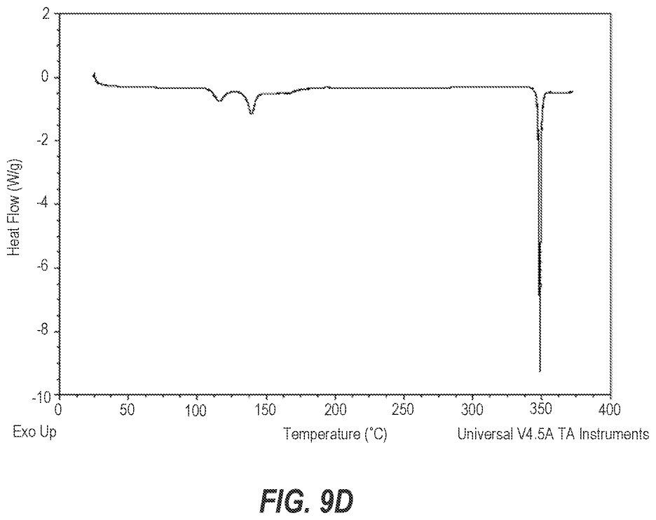

Thus, in some embodiments, Compound 1 EtOH solvate Form A is substantially crystalline. In some embodiments, Compound 1 EtOH solvate Form A is substantially pure crystalline. In some embodiments, Compound 1 EtOH solvate Form A is characterized by an X-ray powder diffractogram generated by an X-ray powder diffraction analysis with an incident beam of Cu Kα radiation. FIG. 9A provides an X-ray powder diffractogram of Compound 1 EtOH solvate Form A at room temperature.

In some embodiments, Compound 1 EtOH solvate Form A is characterized by an X-ray powder diffractogram having a signal at one or more of 20.2±0.2 degrees two-theta, 20.7±0.2 degrees two-theta, and 23.4±0.2 degrees two-theta. In some embodiments, Compound 1 EtOH solvate Form A is characterized by an X-ray powder diffractogram having signals at 20.2±0.2 degrees two-theta, 20.7±0.2 degrees two-theta, and 23.4±0.2 degrees two-theta. In some embodiments, Compound 1 EtOH solvate Form A is characterized by an X-ray powder diffractogram having (a) signals at 20.2±0.2 degrees two-theta, 20.7±0.2 degrees two-theta, and 23.4±0.2 degrees two-theta; and (b) at least one, at least two, or at least three signals selected from 7.5±0.2 degrees two-theta, 12.0±0.2 degrees two-theta, 12.6±0.2 degrees two-theta, 13.8±0.2 degrees two-theta, 15.9±0.2 degrees two-theta, 16.6±0.2 degrees two-theta, 17.1±0.2 degrees two-theta, 18.2±0.2 degrees two-theta, 18.9±0.2 degrees two-theta, 19.8±0.2 degrees two-theta, 21.0±0.2 degrees two-theta, 21.4±0.2 degrees two-theta, 22.4±0.2 degrees two-theta, 22.9±0.2 degrees two-theta, 24.6±0.2 degrees two-theta, 26.4±0.2 degrees two-theta, 26.7±0.2 degrees two-theta, 28.6±0.2 degrees two-theta, 29.2±0.2 degrees two-theta, and 29.6±0.2 degrees two-theta.

In some embodiments, Compound 1 EtOH solvate Form A is characterized by an X-ray powder diffractogram substantially similar to FIG. 9A.

In some embodiments, Compound 1 EtOH solvate Form A is characterized by having a ¹³C ssNMR peak at one or more of 126.6±0.2 ppm, 111.5±0.2 ppm, 57.9±0.2 ppm, 34.4±0.2 ppm, 27.9±0.2 ppm, and 19.0±0.2 ppm. In some embodiments, Compound 1 EtOH solvate Form A is characterized by having a ¹³C ssNMR peak at 126.6±0.2 ppm, 111.5±0.2 ppm, 57.9±0.2 ppm, 34.4±0.2 ppm, 27.9±0.2 ppm, and 19.0±0.2 ppm. In some embodiments, Compound 1 EtOH solvate Form A is characterized by a ¹³C ssNMR spectrum substantially similar to FIG. 9B.

In some embodiments, Compound 1 EtOH solvate Form A is characterized by a TGA thermogram substantially similar to FIG. 9C. In some embodiments, Compound 1 EtOH solvate Form A is characterized by a DSC thermogram substantially similar to FIG. 9D.

Another aspect of the disclosure provides a composition comprising Compound 1 EtOH solvate Form A. In some embodiments, the composition comprises substantially pure crystalline Compound 1 EtOH solvate Form A. In some embodiments, the composition consists essentially of Compound 1 EtOH solvate Form A.

Another aspect of the disclosure provides a method of making Compound 1 EtOH solvate Form A. In some embodiments, Compound 1 EtOH solvate Form A is prepared by:
(a) dissolving Compound 1 in an organic solvent (e.g., THF or THF/water mixture such as THF:H₂O (9:1) to form a first reaction mixture at 55-65° C. (e.g., 60° C.);
(b) adding water to the reaction mixture to precipitate a first solid portion;
(c) isolating the first solid portion and re-suspending the first solid portion in EtOH to form a second reaction mixture; and
(d) isolating a second solid portion from the second reaction mixture and drying the second solid portion to yield Compound 1 EtOH solvate Form A.

10. Compound 1 Tartrate Salt or Cocrystal Form A

In some embodiments, Compound 1 tartrate salt or cocrystal Form A is a crystalline solid comprising crystalline tartrate salt or cocrystal Form A. In some embodiments, the crystalline solid comprises 30% to 99% crystalline Compound 1 tartrate salt or cocrystal Form A relative to the total weight of solid Compound 1. In some embodiments, the crystalline solid comprises 40% to 99% crystalline Compound 1 tartrate salt or cocrystal Form A relative to the total weight of solid Compound 1. In some embodiments, the crystalline solid comprises 50% to 99% crystalline Compound 1 tartrate salt or cocrystal Form A relative to the total weight of solid Compound 1. In some embodiments, the crystalline solid comprises 60% to 99% crystalline Compound 1 tartrate salt or cocrystal Form A relative to the total weight of solid Compound 1. In some embodiments, the crystalline solid comprises 70% to 99% crystalline Compound 1 tartrate salt or cocrystal Form A relative to the total weight of solid Compound 1. In some embodiments, the crystalline solid comprises 75% to 99% crystalline Compound 1 tartrate salt or cocrystal Form A relative to the total weight of solid Compound 1. In some embodiments, the crystalline solid comprises 80% to 99% crystalline Compound 1 tartrate salt or cocrystal Form A relative to the total weight of solid Compound 1. In some embodiments, the crystalline solid comprises 85% to 99% crystalline Compound 1 tartrate salt or cocrystal Form A relative to the total weight of solid Compound 1. In some embodiments, the crystalline solid comprises 90% to 99% crystalline Compound 1 tartrate salt or cocrystal Form A relative to the total weight of solid Compound 1. In some embodiments, the crystalline solid comprises 95% to 99% crystalline Compound 1 tartrate salt or cocrystal Form A relative to the total weight of solid Compound 1.

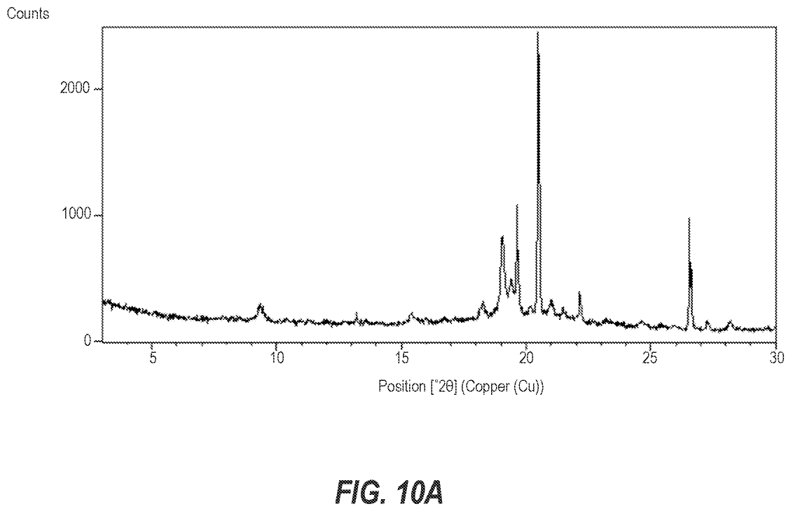

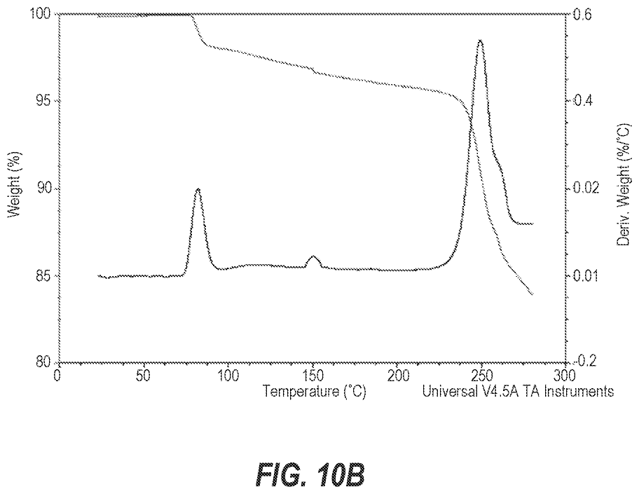

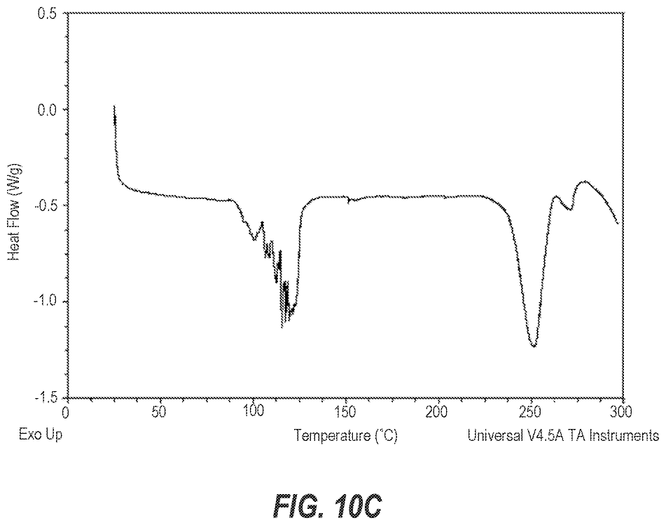

Thus, in some embodiments, Compound 1 tartrate salt or cocrystal Form A is substantially crystalline. In some embodiments, Compound 1 tartrate salt or cocrystal Form A is substantially pure crystalline. In some embodiments, Compound 1 tartrate salt or cocrystal Form A is characterized by an X-ray powder diffractogram generated by an X-ray powder diffraction analysis with an incident beam of Cu Kα radiation. FIG. 10A provides an X-ray powder diffractogram of Compound 1 tartrate salt or cocrystal Form A at room temperature.

In some embodiments, Compound 1 tartrate salt or cocrystal Form A is characterized by an X-ray powder diffractogram having signals at 19.0±0.2 degrees two-theta, 19.6±0.2 degrees two-theta, and 20.5±0.2 degrees two-theta. In some embodiments, Compound 1 tartrate salt or cocrystal Form A is characterized by an X-ray powder diffractogram having (a) signals at 19.0±0.2 degrees two-theta, 19.6±0.2 degrees two-theta, and 20.5±0.2 degrees two-theta; and (b) at least one, at least two, or at least three signals selected from 19.4±0.2 degrees two-theta, 22.1±0.2 degrees two-theta, 26.5±0.2 degrees two-theta, and 26.6±0.2 degrees two-theta.

In some embodiments, Compound 1 tartrate salt or cocrystal Form A is characterized by an X-ray powder diffractogram substantially similar to FIG. 10A.

In some embodiments, Compound 1 tartrate salt or cocrystal Form A is characterized by a TGA thermogram substantially similar to FIG. 10B. In some embodiments, Compound 1 tartrate salt or cocrystal Form A is characterized by a DSC thermogram substantially similar to FIG. 10C.

Another aspect of the disclosure provides a composition comprising Compound 1 tartrate salt or cocrystal Form A. In some embodiments, the composition comprises substantially pure crystalline Compound 1 tartrate salt or cocrystal Form A. In some embodiments, the composition consists essentially of Compound 1 tartrate salt or cocrystal Form A.

Another aspect of the disclosure provides a method of making Compound 1 tartrate salt or cocrystal Form A. In some embodiments, Compound 1 tartrate salt or cocrystal Form A is prepared by:
(a) reacting Compound 1 Form A with tartaric acid in the presence of a second base (e.g., NaOH) and THF/water (e.g., 9:1 v/v) to form a reaction mixture; and
(b) evaporating a solid portion from the reaction mixture to yield Compound 1 tartrate salt or cocrystal Form A.

11. Compound 1 Tartrate Salt or Cocrystal Form B

In some embodiments, Compound 1 tartrate salt or cocrystal Form B is a crystalline solid comprising crystalline tartrate salt or cocrystal Form B. In some embodiments, the crystalline solid comprises 30% to 99% crystalline Compound 1 tartrate salt or cocrystal Form B relative to the total weight of solid Compound 1. In some embodiments, the crystalline solid comprises 40% to 99% crystalline Compound 1 tartrate salt or cocrystal Form B relative to the total weight of solid Compound 1. In some embodiments, the crystalline solid comprises 50% to 99% crystalline Compound 1 tartrate salt or cocrystal Form B relative to the total weight of solid Compound 1. In some embodiments, the crystalline solid comprises 60% to 99% crystalline Compound 1 tartrate salt or cocrystal Form B relative to the total weight of solid Compound 1. In some embodiments, the crystalline solid comprises 70% to 99% crystalline Compound 1 tartrate salt or cocrystal Form B relative to the total weight of solid Compound 1. In some embodiments, the crystalline solid comprises 75% to 99% crystalline Compound 1 tartrate salt or cocrystal Form B relative to the total weight of solid Compound 1. In some embodiments, the crystalline solid comprises 80% to 99% crystalline Compound 1 tartrate salt or cocrystal Form B relative to the total weight of solid Compound 1. In some embodiments, the crystalline solid comprises 85% to 99% crystalline Compound 1 tartrate salt or cocrystal Form B relative to the total weight of solid Compound 1. In some embodiments, the crystalline solid comprises 90% to 99% crystalline Compound 1 tartrate salt or cocrystal Form B relative to the total weight of solid Compound 1. In some embodiments, the crystalline solid comprises 95% to 99% crystalline Compound 1 tartrate salt or cocrystal Form B relative to the total weight of solid Compound 1.

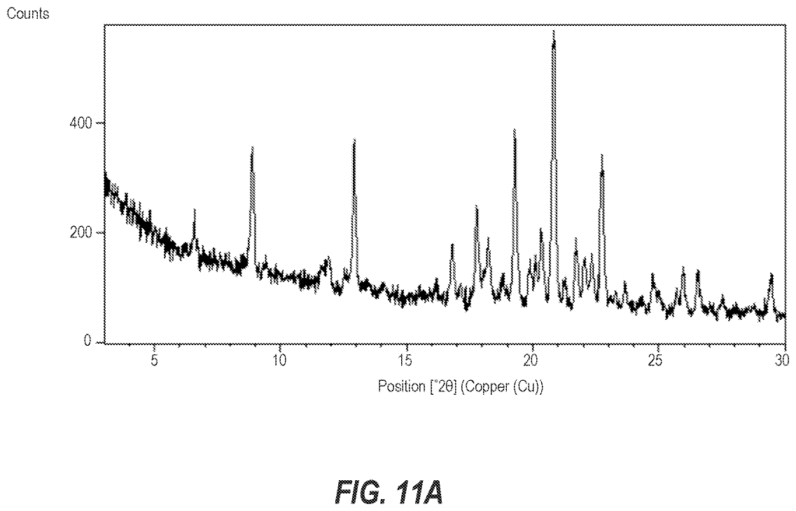

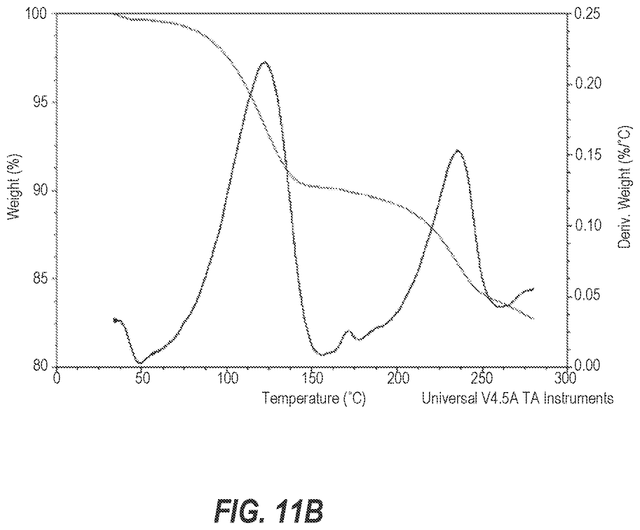

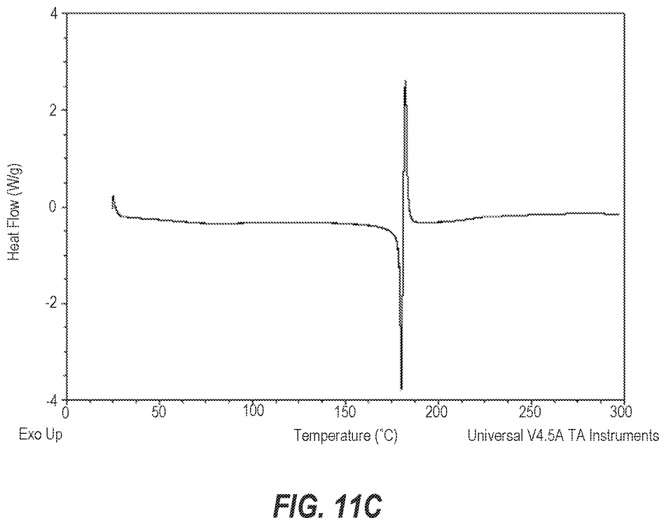

Thus, in some embodiments, Compound 1 tartrate salt or cocrystal Form B is substantially crystalline. In some embodiments, Compound 1 tartrate salt or cocrystal Form B is substantially pure crystalline. In some embodiments, Compound 1 tartrate salt or cocrystal Form B is characterized by an X-ray powder diffractogram generated by an X-ray powder diffraction analysis with an incident beam of Cu Kα radiation. FIG. 11A provides an X-ray powder diffractogram of Compound 1 tartrate salt or cocrystal Form B at room temperature.

In some embodiments, Compound 1 tartrate salt or cocrystal Form B is characterized by an X-ray powder diffractogram having signals at 8.9±0.2 degrees two-theta, 17.8±0.2 degrees two-theta and 22.7±0.2 degrees two-theta. In some embodiments, Compound 1 tartrate salt or cocrystal Form B is characterized by an X-ray powder diffractogram having (a) signals at 8.9±0.2 degrees two-theta, 17.8±0.2 degrees two-theta, and 22.7±0.2 degrees two-theta; and (b) at least one, at least two, or at least three signals selected from 6.6±0.2 degrees two-theta, 11.9±0.2 degrees two-theta, 12.9±0.2 degrees two-theta, 16.8±0.2 degrees two-theta, 18.2±0.2 degrees two-theta, 18.8±0.2 degrees two-theta, 19.3±0.2 degrees two-theta, 19.8±0.2 degrees two-theta, 20.1±0.2 degrees two-theta, 20.3±0.2 degrees two-theta, 20.8±0.2 degrees two-theta, 21.7±0.2 degrees two-theta, 22.0±0.2 degrees two-theta, 22.3±0.2 degrees two-theta, 24.7±0.2 degrees two-theta, 26.0±0.2 degrees two-theta, 26.5±0.2 degrees two-theta, and 23.6±0.2 degrees two-theta, and 29.5±0.2 degrees two-theta.

In some embodiments, Compound 1 tartrate salt or cocrystal Form B is characterized by an X-ray powder diffractogram substantially similar to FIG. 11A.

In some embodiments, Compound 1 tartrate salt or cocrystal Form B is characterized by a TGA thermogram substantially similar to FIG. 11B. In some embodiments, Compound 1 tartrate salt or cocrystal Form B is characterized by a DSC thermogram substantially similar to FIG. 11C.

Another aspect of the disclosure provides a composition comprising Compound 1 tartrate salt or cocrystal Form B. In some embodiments, the composition comprises substantially pure crystalline Compound 1 tartrate salt or cocrystal Form B. In some embodiments, the composition consists essentially of Compound 1 tartrate salt or cocrystal Form B.

Another aspect of the disclosure provides a method of making Compound 1 tartrate salt or cocrystal Form B. In some embodiments, Compound 1 tartrate salt or cocrystal Form B is prepared by:
(a) reacting Compound 1 Form A with tartaric acid in the presence of a second base (e.g., Ca(OH)₂) and ethyl acetate to form a reaction mixture; and
(b) evaporating a solid portion from the reaction mixture to yield Compound 1 tartrate salt or cocrystal Form B.

12. Compound 1 Tartrate Salt or Cocrystal Form C

In some embodiments, Compound 1 tartrate salt or cocrystal Form C is a crystalline solid comprising crystalline tartrate salt or cocrystal Form C. In some embodiments, the crystalline solid comprises 30% to 99% crystalline Compound 1 tartrate salt or cocrystal Form C relative to the total weight of solid Compound 1. In some embodiments, the crystalline solid comprises 40% to 99% crystalline Compound 1 tartrate salt or cocrystal Form C relative to the total weight of solid Compound 1. In some embodiments, the crystalline solid comprises 50% to 99% crystalline Compound 1 tartrate salt or cocrystal Form C relative to the total weight of solid Compound 1. In some embodiments, the crystalline solid comprises 60% to 99% crystalline Compound 1 tartrate salt or cocrystal Form C relative to the total weight of solid Compound 1. In some embodiments, the crystalline solid comprises 70% to 99% crystalline Compound 1 tartrate salt or cocrystal Form C relative to the total weight of solid Compound 1. In some embodiments, the crystalline solid comprises 75% to 99% crystalline Compound 1 tartrate salt or cocrystal Form C relative to the total weight of solid Compound 1. In some embodiments, the crystalline solid comprises 80% to 99% crystalline Compound 1 tartrate salt or cocrystal Form C relative to the total weight of solid Compound 1. In some embodiments, the crystalline solid comprises 85% to 99% crystalline Compound 1 tartrate salt or cocrystal Form C relative to the total weight of solid Compound 1. In some embodiments, the crystalline solid comprises 90% to 99% crystalline Compound 1 tartrate salt or cocrystal Form C relative to the total weight of solid Compound 1. In some embodiments, the crystalline solid comprises 95% to 99% crystalline Compound 1 tartrate salt or cocrystal Form C relative to the total weight of solid Compound 1.

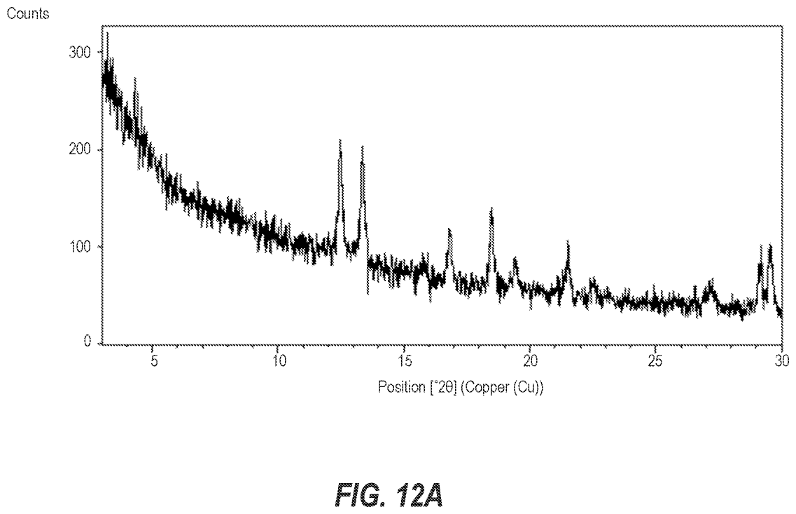

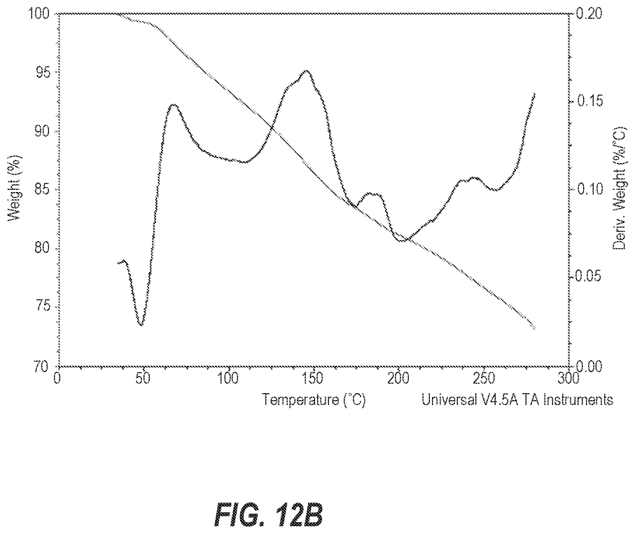

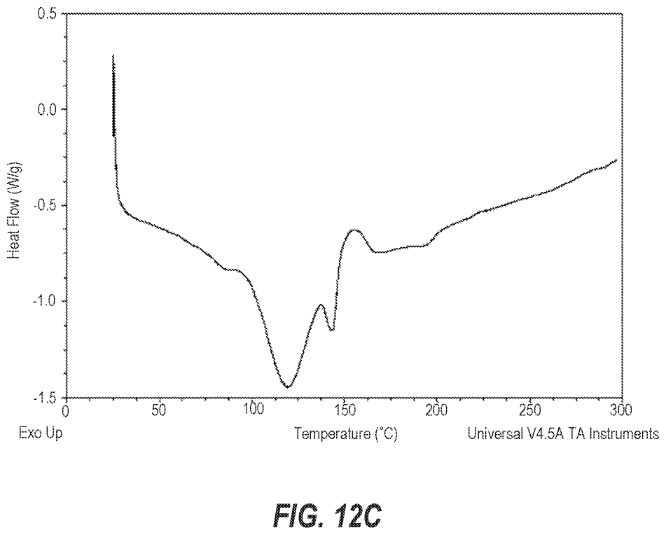

Thus, in some embodiments, Compound 1 tartrate salt or cocrystal Form C is substantially crystalline. In some embodiments, Compound 1 tartrate salt or cocrystal Form C is substantially pure crystalline. In some embodiments, Compound 1 tartrate salt or cocrystal Form C is characterized by an X-ray powder diffractogram generated by an X-ray powder diffraction analysis with an incident beam of Cu Kα radiation. FIG. 12A provides an X-ray powder diffractogram of Compound 1 tartrate salt or cocrystal Form C at room temperature.

In some embodiments, Compound 1 tartrate salt or cocrystal Form C is characterized by an X-ray powder diffractogram having signals at 12.4±0.2 degrees two-theta, 13.3±0.2 degrees two-theta, and 18.5±0.2 degrees two-theta. In some embodiments, Compound 1 tartrate salt or cocrystal Form C is characterized by an X-ray powder diffractogram having (a) signals at 12.4±0.2 degrees two-theta, 13.3±0.2 degrees two-theta, and 18.5±0.2 degrees two-theta; and (b) at least one, at least two, or at least three signals selected from 15.8±0.2 degrees two-theta, 16.8±0.2 degrees two-theta, 19.4±0.2 degrees two-theta, 21.5±0.2 degrees two-theta, 22.5±0.2 degrees two-theta, 27.1±0.2 degrees two-theta, 29.2±0.2 degrees two-theta, and 29.5±0.2 degrees two-theta.

In some embodiments, Compound 1 tartrate salt or cocrystal Form C is characterized by an X-ray powder diffractogram substantially similar to FIG. 12A.

In some embodiments, Compound 1 tartrate salt or cocrystal Form C is characterized by a TGA thermogram substantially similar to FIG. 12B. In some embodiments, Compound 1 tartrate salt or cocrystal Form C is characterized by a DSC thermogram substantially similar to FIG. 12C.

Another aspect of the disclosure provides a composition comprising Compound 1 tartrate salt or cocrystal Form C. In some embodiments, the composition comprises substantially pure crystalline Compound 1 tartrate salt or cocrystal Form C. In some embodiments, the composition consists essentially of Compound 1 tartrate salt or cocrystal Form C.

Another aspect of the disclosure provides a method of making Compound 1 tartrate salt or cocrystal Form C. In some embodiments, Compound 1 tartrate salt or cocrystal Form C is prepared by:
(a) reacting Compound 1 Form A with tartaric acid in the presence of Ca(OH)₂ and THF (e.g., THF/water mixture at 9:1, v:v) to form a reaction mixture; and
(b) evaporating a solid portion from the reaction mixture to yield Compound 1 tartrate salt or cocrystal Form C.

13. Compound 1 Tartrate Salt or Cocrystal Form D

In some embodiments, Compound 1 tartrate salt or cocrystal Form D is a crystalline solid comprising crystalline tartrate salt or cocrystal Form D. In some embodiments, the crystalline solid comprises 30% to 99% crystalline Compound 1 tartrate salt or cocrystal Form D relative to the total weight of solid Compound 1. In some embodiments, the crystalline solid comprises 40% to 99% crystalline Compound 1 tartrate salt or cocrystal Form D relative to the total weight of solid Compound 1. In some embodiments, the crystalline solid comprises 50% to 99% crystalline Compound 1 tartrate salt or cocrystal Form D relative to the total weight of solid Compound 1. In some embodiments, the crystalline solid comprises 60% to 99% crystalline Compound 1 tartrate salt or cocrystal Form D relative to the total weight of solid Compound 1. In some embodiments, the crystalline solid comprises 70% to 99% crystalline Compound 1 tartrate salt or cocrystal Form D relative to the total weight of solid Compound 1. In some embodiments, the crystalline solid comprises 75% to 99% crystalline Compound 1 tartrate salt or cocrystal Form D relative to the total weight of solid Compound 1. In some embodiments, the crystalline solid comprises 80% to 99% crystalline Compound 1 tartrate salt or cocrystal Form D relative to the total weight of solid Compound 1. In some embodiments, the crystalline solid comprises 85% to 99% crystalline Compound 1 tartrate salt or cocrystal Form D relative to the total weight of solid Compound 1. In some embodiments, the crystalline solid comprises 90% to 99% crystalline Compound 1 tartrate salt or cocrystal Form D relative to the total weight of solid Compound 1. In some embodiments, the crystalline solid comprises 95% to 99% crystalline Compound 1 tartrate salt or cocrystal Form D relative to the total weight of solid Compound 1.

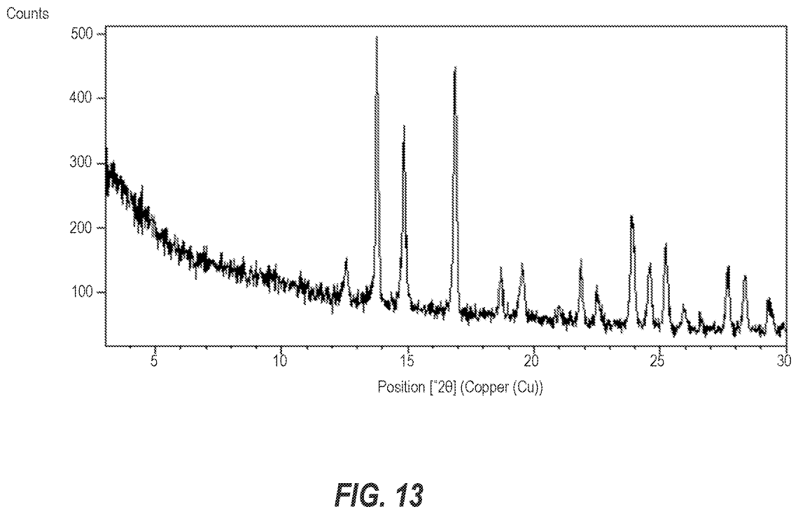

Thus, in some embodiments, Compound 1 tartrate salt or cocrystal Form D is substantially crystalline. In some embodiments, Compound 1 tartrate salt or cocrystal Form D is substantially pure crystalline. In some embodiments, Compound 1 tartrate salt or cocrystal Form D is characterized by an X-ray powder diffractogram generated by an X-ray powder diffraction analysis with an incident beam of Cu Kα radiation. FIG. 13 provides an X-ray powder diffractogram of Compound 1 tartrate salt or cocrystal Form D at room temperature.

In some embodiments, Compound 1 tartrate salt or cocrystal Form D is characterized by an X-ray powder diffractogram having a signal at one or more of 13.8±0.2 degrees two-theta, 14.8±0.2 degrees two-theta, and 25.2±0.2 degrees two-theta. In some embodiments, Compound 1 tartrate salt or cocrystal Form D is characterized by an X-ray powder diffractogram having signals at 13.8±0.2 degrees two-theta, 14.8±0.2 degrees two-theta, and 25.2±0.2 degrees two-theta. In some embodiments, Compound 1 tartrate salt or cocrystal Form D is characterized by an X-ray powder diffractogram having (a) signals at 13.8±0.2 degrees two-theta, 14.8±0.2 degrees two-theta, and 25.2±0.2 degrees two-theta; and (b) at least one, at least two, or at least three signals selected from 12.5±0.2 degrees two-theta, 18.7±0.2 degrees two-theta, 19.5±0.2 degrees two-theta, 21.9±0.2 degrees two-theta, 22.5±0.2 degrees two-theta, 23.9±0.2 degrees two-theta, 24.5±0.2 degrees two-theta, 27.7±0.2 degrees two-theta, and 28.3±0.2 degrees two-theta.

In some embodiments, Compound 1 tartrate salt or cocrystal Form D is characterized by an X-ray powder diffractogram substantially similar to FIG. 13.

Another aspect of the disclosure provides a composition comprising Compound 1 tartrate salt or cocrystal Form D. In some embodiments, the composition comprises substantially pure crystalline Compound 1 tartrate salt or cocrystal Form D. In some embodiments, the composition consists essentially of Compound 1 tartrate salt or cocrystal Form D.

Another aspect of the disclosure provides a method of making Compound 1 tartrate salt or cocrystal Form D. In some embodiments, Compound 1 tartrate salt or cocrystal Form D is prepared by:
(a) reacting Compound 1 Form A with tartaric acid in the presence of Mg(OH)₂ and THF (e.g., THF/water mixture at 9:1, v:v) to form a reaction mixture; and
(b) evaporating a solid portion from the reaction mixture to yield Compound 1 tartrate salt or cocrystal Form D.

### III. Solid Dispersions of Compound 1

In another aspect, the disclosure features a solid dispersion comprising at least one a solid form of Compound 1 or a salt, or a solvate, or a cocrystal thereof, including any one or more of the solid forms described herein and as described in International Patent Application No. PCT/US2020/032832, and a polymer carrier. The solid dispersions of this disclosure are prepared by dissolving the solid form of Compound 1 or a pharmaceutically acceptable salt thereof in a solvent system having specific weight or volume ratios or ranges thereof among the various solvents in the system. Without wishing to be bound by theory, the inventors have found that the solvent ratios described herein lead to improved solubility and stability of the drug in the dispersion and/or more desirable spray drying process space, whereby a broader range of feed rates (e.g., 15-45 kg/h vs. about 20-34 kg/h) may be explored. The benefit of a broader range of feed rates during the spray drying process is that it allows the inventors to determine whether there are any changes to various material properties of the SDD (e.g., particle size, powder density, surface morphology, crystallinity) as the process for making the SDD's are being scaled up.

In some embodiments, a solid dispersion of this disclosure is prepared by dissolving one or more solid forms of Compound 1 or a salt, or a solvate, or a cocrystal thereof in a solvent system comprising a first organic solvent, a second organic solvent, and optionally water, wherein when water is absent from the solvent system, the volume ratio of the first organic solvent to the second organic solvent is between about 55/45 v/v and about 90/10 v/v (e.g., about 55/45 v/v, about 60/40 v/v, about 65/35 v/v, about 70/30 v/v, about 75/25 v/v, about 80/20 v/v, about 85/15 v/v, or about 90/10 v/v); and wherein when water is present in the solvent system, the weight ratio of the first organic solvent to the second organic solvent and to water is between about 55/35/10 w/w and about 80/10/10 (e.g., about 55/35/10, about 56/34/10, about 57/34/9, about 60/30/10, about 60/31/9, about 65/25/10, about 65/26/9, about 70/20/10, about 70/21/9, about 75/15/10, about 75/16/9, about 80/10/10, or about 80/11/9) or about 55/35/10 w/w and about 65/34.5/0.5 w/w; wherein when the weight ratio of the first organic solvent to the second organic solvent and to water is about 55/35/10 w/w, the solid dispersion comprises greater than about 50% w/w of the solid form of Compound 1 or a pharmaceutically acceptable salt thereof. In some embodiments, when water is absent from the solvent system, the volume ratio of the first organic solvent to the second organic solvent is between about 60/40 v/v and about 80/20 v/v. In some embodiments, when water is absent from the solvent system, the volume ratio of the first organic solvent to the second organic solvent is about 60/40 v/v or about 80/20 v/v; and when water is present in the solvent system, the weight ratio of the first organic solvent to the second organic solvent and to water is about 56.8/33.7/9.5 w/w or about 75/15/10 w/w; wherein when the weight ratio of the first organic solvent to the second organic solvent and to water is about 56.8/33.7/9.5 w/w, the solid dispersion comprises higher than about 50% w/w of the solid form of Compound 1 or a pharmaceutically acceptable salt thereof.

In some embodiments, a solid dispersion of this disclosure comprises no lower than about 50% w/w of a solid form of Compound 1 or a salt, or a solvate, or a cocrystal thereof; and wherein when the weight ratio of the first organic solvent to the second organic solvent and to water is about 55/35/10 w/w or about 56.8/33.7/9.5 w/w, the solid dispersion comprises higher than about 50% w/w of a solid form of Compound 1 or a salt, or a solvate, or a cocrystal thereof. In some embodiments, a solid dispersion of this disclosure comprises between no lower than about 50% w/w and about 80% w/w of a solid form of Compound 1 or a salt, or a solvate, or a cocrystal thereof; and wherein when the weight ratio of the first organic solvent to the second organic solvent and to water is about 55/35/10 w/w or about 56.8/33.7/9.5 w/w, the solid dispersion comprises greater than 50% w/w of a solid form of Compound 1 or a salt, or a solvate, or a cocrystal thereof. In some embodiments, a solid dispersion of this disclosure comprises between no lower than about 50% w/w and about 80% w/w of a solid form of Compound 1 or a salt, or a solvate, or a cocrystal thereof; and wherein when the weight ratio of the first organic solvent to the second organic solvent and to water is about 55/35/10 w/w or about 56.8/33.7/9.5 w/w, the solid dispersion comprises greater than 50% w/w of a solid form of Compound 1 or a salt, or a solvate, or a cocrystal thereof. In some embodiments, a solid dispersion of this disclosure comprises about 50% w/w and about 80% w/w of the solid form of Compound 1 or a pharmaceutically acceptable salt thereof; and wherein when the weight ratio of the first organic solvent to the second organic solvent and to water is about 55/35/10 w/w or about 56.8/33.7/9.5 w/w, the solid dispersion comprises about 80% w/w of a solid form of Compound 1 or a salt, or a solvate, or a cocrystal thereof.

In some embodiments, the first organic solvent in the solvent system used to prepare a solid dispersion of this disclosure is a polar aprotic solvent. Non-limiting examples of suitable first organic solvents are dichloromethane (DCM), tetrahydrofuran (THF), 2-methyltetrahydrofuran (Me-THF), ethyl acetate (EtOAc), acetone, acetonitrile (MeCN), and dimethylformamide (DMF). In some embodiments, the first organic solvent is selected from DCM, THF, and Me-THF. In some embodiments, the second organic solvent in the solvent system used to prepare a solid dispersion of this disclosure is an alcohol. Non-limiting examples of suitable first organic solvents are methanol (MeOH), ethanol (EtOH), n-butanol, tert-butanol, isopropyl alcohol (IPA), and 2-propanol. In some embodiments, the second organic solvent is MeOH or EtOH. Other suitable exemplary solvents are as described in International Patent Application No. WO 2011/119984, which is incorporated herein by reference in its entirety.

In some embodiments, the polymer is hydroxypropylmethylcellulose acetate succinate (HPMCAS). In another embodiment, the polymer is polyvinylpyrrolidone/vinyl acetate PVPVA. In another embodiment, the polymer is hydroxypropylmethylcellulose (HPMC). Other suitable exemplary polymers are as described in International Patent Application No. WO 2011/119984.

In some embodiments, the polymer is present in an amount from about 0.1% by weight to about 10% by weight based on the total weight of the dispersion (prior to drying or solidifying). In another embodiment, the polymer is present in an amount from about 0.2% by weight to about 7.5% by weight based on the total weight of the dispersion (prior to drying or solidifying). In another embodiment, the polymer is present in an amount from about 0.2% by weight to about 5.0% by weight based on the total weight of the dispersion (prior to drying or solidifying).

In another aspect, the disclosure features a pharmaceutical composition comprising the solid dispersion and a pharmaceutically acceptable carrier. In some embodiments, the disclosure features a pharmaceutical composition comprising spray-dried, neat substantially amorphous Compound 1 without polymer.

Methods of Preparing Solid Dispersions of Compound 1

Starting from Compound 1 or a salt, solvate, or cocrystal of that compound, the solid dispersion comprising a solid form of Compound 1 or a salt, or a solvate, or a cocrystal thereof. Compound 1 or a salt, or a solvate, or a cocrystal thereof may be prepared by rotary evaporation or by spray dry methods. Some embodiments of the disclosure provide a pharmaceutical composition comprising a solid form of Compound 1 or a salt, or a solvate, or a cocrystal thereof. In some embodiments, the composition comprising a solid form of Compound 1 or a salt, or a solvate, or a cocrystal thereof is a spray-dried dispersion.

In some embodiments, a solid dispersion of this disclosure is prepared by dissolving Compound 1, a salt, solvate, or cocrystal thereof in an appropriate solvent system and at weight or volume ratios or ranges thereof as described above and rotary evaporating the solvent mixture to leave a foam produces the amorphous form. In some embodiments, a warm water bath is used to expedite the evaporation.

Solid dispersions may also be prepared from any of Compound 1 and salts, solvates and cocrystals of Compound 1, using spray dry methods. Spray drying is a process that converts a liquid feed to a dried particulate form. Optionally, a secondary drying process such as fluidized bed drying or vacuum drying, may be used to reduce residual solvents to pharmaceutically acceptable levels. Typically, spray drying involves contacting a highly dispersed liquid suspension or solution, and a sufficient volume of hot air to produce evaporation and drying of the liquid droplets. The preparation to be spray dried can be any solution, coarse suspension, slurry, colloidal dispersion, or paste that may be atomized using the selected spray drying apparatus. In a standard procedure, the preparation is sprayed into a current of warm filtered air that evaporates the solvent and conveys the dried product to a collector (e.g., a cyclone). The spent air is then exhausted with the solvent, or alternatively the spent air is sent to a condenser to capture and potentially recycle the solvent. Commercially available types of apparatus may be used to conduct the spray drying. For example, commercial spray dryers are manufactured by Buchi Ltd. And Niro (e.g., the PSD line of spray driers manufactured by Niro) (see, US 2004/0105820; US 2003/0144257).

Spray drying typically employs solid loads of material from about 3% to about 30% by weight, (i.e., drug and excipients), for example, about 4% to about 20% by weight, at least about 10%. In general, the upper limit of solid loads is governed by the viscosity of (e.g., the ability to pump) the resulting solution and the solubility of the components in the solution. Generally, the viscosity of the solution can determine the size of the particle in the resulting powder product.

Techniques and methods for spray drying may be found in Perry's Chemical Engineering Handbook, 6th Ed., R. H. Perry, D. W. Green & J. O. Maloney, eds., McGraw-Hill book co. (1984); and Marshall “Atomization and Spray-Drying” 50, Chem. Eng. Prog. Monogr. Series 2 (1954). In general, the spray drying is conducted with an inlet temperature of from about 60° C. to about 200° C., for example, from about 95° C. to about 185° C., from about 110° C. to about 182° C., from about 96° C. to about 180° C., e.g., about 145° C. The spray drying is generally conducted with an outlet temperature of from about 30° C. to about 90° C., for example from about 40° C. to about 80° C., about 45° C. to about 80° C., e.g., about 75° C. The atomization flow rate is generally from about 4 kg/h to about 12 kg/h, for example, from about 4.3 kg/h to about 10.5 kg/h, e.g., about 6 kg/h or about 10.5 kg/h. The feed flow rate is generally from about 3 kg/h to about 10 kg/h, for example, from about 3.5 kg/h to about 9.0 kg/h, e.g., about 8 kg/h or about 7.1 kg/h. The atomization ratio is generally from about 0.3 to 1.7, e.g., from about 0.5 to 1.5, e.g., about 0.8 or about 1.5.

Removal of the solvent may require a subsequent drying step, such as tray drying, fluid bed drying (e.g., from about room temperature to about 100° C.), vacuum drying, microwave drying, rotary drum drying, or biconical vacuum drying (e.g., from about room temperature to about 200° C.).

In another aspect, the disclosure features a method of preparing a solid dispersion comprising a solid form of 4-(5-(4-fluorophenyl)-6-(tetrahydro-2H-pyran-4-yl)-1,5-dihydropyrrolo[2,3-f]indazol-7-yl)benzoic acid (Compound 1) or a salt, or a solvate, or a cocrystal thereof and a polymer carrier, the method comprising:
(a) dissolving the solid form of Compound 1 or a salt, or a solvate, or a cocrystal thereof in a solvent system comprising a first organic solvent, a second organic solvent, and optionally water to form a reaction mixture;
(b) adding the polymer carrier to the reaction mixture and stirring the reaction mixture at ambient temperature; and
(c) spray drying the reaction mixture to yield the solid dispersion as a final product; wherein: when water is absent from the solvent system, the volume ratio of the first organic solvent to the second organic solvent is between about 55/45 v/v and about 90/10 v/v; and when water is present in the solvent system, the weight ratio of the first organic solvent to the second organic solvent and to water is between about 55/35/10 w/w and about 80/10/10; and wherein when the weight ratio of the first organic solvent to the second organic solvent and to water is about 55/35/10 w/w, the solid dispersion as a final product comprises greater than about 50% w/w of the solid form of Compound 1 or a salt, or a solvate, or a cocrystal thereof. Further exemplary weight and volume ratios or ranges thereof in the solvent system, as well as types of solvents, polymers, and reaction conditions are as described above.

### IV. Novel AAT Modulators

A further aspect of the disclosure is directed to Compound 2 and Compound 3:

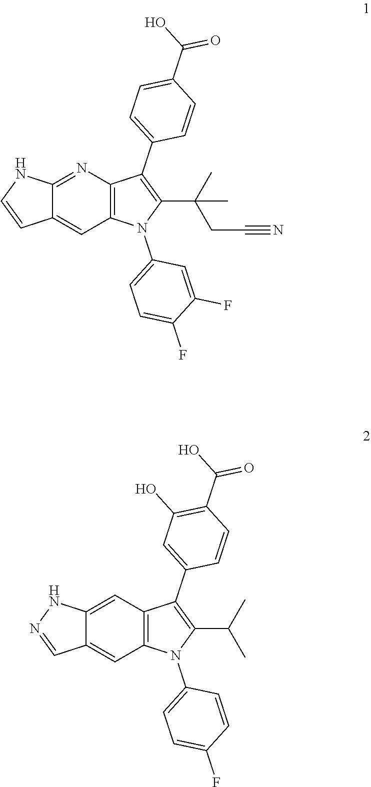 [SMILES: CC(C)(CC#N)c1c(-c2ccc(C(=O)O)cc2)c2nc3[nH]ccc3cc2n1-c1ccc(F)c(F)c1 ; CC(C)c1c(-c2ccc(C(=O)O)c(O)c2)c2cc3[nH]ncc3cc2n1-c1ccc(F)cc1]

a tautomer thereof, a deuterated derivative of that compound or tautomer, or a pharmaceutically acceptable salt of the foregoing. Methods of preparing these compounds are described in Example 4.

### V. Pharmaceutical Compositions

A pharmaceutical composition of the disclosure may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include at least one other active agent. Alternatively, a pharmaceutical composition comprising a solid dispersion and/or one or more of the solid forms disclosed herein can be administered as a separate composition concurrently with, prior to, or subsequent to, a composition comprising at least one additional active agent.

As described above, pharmaceutical compositions of the disclosure may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose, and cellulose acetate), powdered tragacanth, malt, gelatin, talc, excipients (such as cocoa butter and suppository waxes), oils (such as peanut oil, cottonseed oil, safflower oil, sesame oil, olive oil, corn oil, and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### VI. Methods of Treatment and Medical Uses

In another aspect of the disclosure, the solid forms of Compound 1 disclosed herein, and Compounds 2 and 3 (or a tautomer thereof, a deuterated derivative of that compound or tautomer, or a pharmaceutically acceptable salt of the foregoing), and the pharmaceutical compositions comprising any one or more of the foregoing, may be used to treat AATD. In some embodiments, the subject in need of treatment with the compounds and compositions of the disclosure carries the ZZ mutation. In some embodiments, the subject in need of treatment with the compounds and compositions of the disclosure carries the SZ mutation.

In another aspect of the disclosure, the methods of treating AATD comprise administering to a patient in need a solid form of Compound 1. In some embodiments, the solid form of Compound 1 administered in the method of treating AATD is selected from Compound 1 neat Form C, Compound 1 Na salt Form A, Compound 1 Na salt Form B, Compound 1 Na salt Form C, Compound 1 Na salt Form D, Compound 1 Ca salt Form A, Compound 1 HCl salt Form A, Compound 1 DMSO solvate Form A, Compound 1 EtOH solvate Form A, Compound 1 tartrate salt or cocrystal Form A, Compound 1 tartrate salt or cocrystal Form B, Compound 1 tartrate salt or cocrystal Form C, and Compound 1 tartrate salt or cocrystal Form D. In some embodiments, said patient in need thereof has a Z mutation in the alpha-1 antitrypsin gene. In another aspect of the disclosure, the methods of treating AATD comprise administering to a patient in need Compound 2 or Compound 3 (or a tautomer thereof, a deuterated derivative of that compound or tautomer, or a pharmaceutically acceptable salt of the foregoing). In some embodiments, the methods of treating AATD comprise administering to a patient in need a solid dispersion comprising a solid form of Compound 1 or a salt, solvate, or cocrystal thereof. In some embodiments, the methods of treating AATD comprise administering to a patient in need a spray dried dispersion comprising a solid form of Compound 1 or a salt, solvate, or cocrystal thereof. In some embodiments, said patient in need thereof is homozygous for the Z-mutation in the alpha-1 antitrypsin gene.

Another aspect of the disclosure provides use of a solid form of Compound 1, or Compound 2 or Compound 3 (or a tautomer thereof, a deuterated derivative of that compound or tautomer, or a pharmaceutically acceptable salt of the foregoing), for the manufacture of a medicament for treating AATD. In some embodiments, the solid form of Compound 1 selected from Compound 1 neat Form C, Compound 1 Na salt Form A, Compound 1 Na salt Form B, Compound 1 Na salt Form C, Compound 1 Na salt Form D, Compound 1 Ca salt Form A, Compound 1 HCl salt Form A, Compound 1 DMSO solvate Form A, Compound 1 EtOH solvate Form A, Compound 1 tartrate salt or cocrystal Form A, Compound 1 tartrate salt or cocrystal Form B, Compound 1 tartrate salt or cocrystal Form C, and Compound 1 tartrate salt or cocrystal Form D. In some embodiments, the disclosure provides use of a solid dispersion comprising a solid form of Compound 1 or a salt, solvate, or cocrystal thereof for the manufacture of a medicament for treating AATD. In some embodiments, the disclosure provides use of a spray dried dispersion comprising a solid form of Compound 1 or a salt, solvate, or cocrystal thereof for the manufacture of a medicament for treating AATD.

Yet another aspect of the disclosure provides methods of modulating alpha-1 antitrypsin (AAT) activity comprising the step of contacting said alpha-1-antitrypsin with a solid form of Compound 1, or Compound 2 or Compound 3 (or a tautomer thereof, a deuterated derivative of that compound or tautomer, or a pharmaceutically acceptable salt of the foregoing). In some embodiments, the solid form of Compound 1 used in the method of modulating AAT activity is selected from Compound 1 neat Form C, Compound 1 Na salt Form A, Compound 1 Na salt Form B, Compound 1 Na salt Form C, Compound 1 Na salt Form D, Compound 1 Ca salt Form A, Compound 1 HCl salt Form A, Compound 1 DMSO solvate Form A, Compound 1 EtOH solvate Form A, Compound 1 tartrate salt or cocrystal Form A, Compound 1 tartrate salt or cocrystal Form B, Compound 1 tartrate salt or cocrystal Form C, and Compound 1 tartrate salt or cocrystal Form D. In some embodiments, the methods of modulating AAT activity comprise contacting said alpha-1-antitrypsin with a solid dispersion comprising a solid form of Compound 1 or a salt, solvate, or cocrystal thereof. In some embodiments, the methods of modulating AAT activity comprise contacting said alpha-1-antitrypsin with a spray dried dispersion comprising a solid form of Compound 1 or a salt, solvate, or cocrystal thereof.

### VII. Non-Limiting Example Embodiments

Without limitation, some embodiments of this disclosure include:

1. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) neat Form C.

2. The Compound 1 neat Form C according to Embodiment 1, characterized by an X-ray powder diffractogram having a signal at 9.4±0.2 degrees two-theta, and a signal at one or more of 15.4±0.2 degrees two-theta, 19.0±0.2 degrees two-theta, and 21.1±0.2 degrees two-theta.

3. The Compound 1 neat Form C according to Embodiment 1 or 2, characterized by an X-ray powder diffractogram having a signal at 9.4±0.2 degrees two-theta, and signals at two or more of 15.4±0.2 degrees two-theta, 19.0±0.2 degrees two-theta, and 21.1±0.2 degrees two-theta.

4. The Compound 1 neat Form C according to any one of Embodiments 1-3, characterized by an X-ray powder diffractogram having signals at 9.4±0.2 degrees two-theta, 19.0±0.2 degrees two-theta, 15.4±0.2 degrees two-theta, and 21.1±0.2 degrees two-theta.

5. The Compound 1 neat Form C according to any one of Embodiments 1-4, characterized by an X-ray powder diffractogram having (a) signals at 9.4±0.2 degrees two-theta, 15.4±0.2 degrees two-theta, 19.0±0.2 degrees two-theta, and 21.1±0.2 degrees two-theta; and (b) at least one signal selected from 18.2±0.2 degrees two-theta, 19.6±0.2 degrees two-theta, and 20.1±0.2 degrees two-theta.

6. The Compound 1 neat Form C according to any one of Embodiments 1-5, characterized by an X-ray powder diffractogram substantially similar to FIG. 1A.

7. The Compound 1 neat Form C according to any one of Embodiments 1-6, characterized by an ¹⁹F ssNMR peak at −107.5±0.2 ppm.

8. The Compound 1 neat Form C according to any one of Embodiments 1-6, characterized by an ¹⁹F ssNMR spectrum substantially similar to FIG. 1B.

9. A method of making the Compound 1 neat Form C according to any one of Embodiments 1-8, comprising:
(a) contacting Compound 1 with an organic solvent to form a first reaction mixture;
(b) heating and stirring the first reaction mixture; and
(c) isolating a solid portion from step (b) and heating the solid portion in an inert environment to yield Compound 1 neat Form C.

10. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Na salt Form A.

11. The Compound 1 Na salt Form A according to Embodiment 10, characterized by an X-ray powder diffractogram having a signal at at least one of 7.3±0.2 degrees two-theta and 11.6±0.2 degrees two-theta.

12. The Compound 1 Na salt Form A according to Embodiment 10 or 11, characterized by an X-ray powder diffractogram having signals at at least one of 7.3±0.2 degrees two-theta and 11.6±0.2 degrees two-theta, and at least one of 17.8±0.2 degrees two-theta and 20.6±0.2 degrees two-theta.

13. The Compound 1 Na salt Form A according to any one of Embodiments 10-12, characterized by an X-ray powder diffractogram having signals at 7.3±0.2 degrees two-theta and 11.6±0.2 degrees two-theta, and at least one of 17.8±0.2 degrees two-theta and 20.6±0.2 degrees two-theta.

14. The Compound 1 Na salt Form A according to any one of Embodiments 10-13, characterized by an X-ray powder diffractogram having signals at 7.3-10.2 degrees two-theta, 11.6±0.2 degrees two-theta, 17.8±0.2 degrees two-theta, and 20.6±0.2 degrees two-theta.

15. The Compound 1 Na salt Form A according to any one of Embodiments 10-14, characterized by an X-ray powder diffractogram having (a) signals at 7.3±0.2 degrees two-theta, 11.6±0.2 degrees two-theta, 17.8±0.2 degrees two-theta, and 20.6±0.2 degrees two-theta; and (b) at least one signal selected from 16.4±0.2 degrees two-theta, 23.2±0.2 degrees two-theta, 18.7±0.2 degrees two-theta, 21.4±0.2 degrees two-theta, and 21.9±0.2 degrees two-theta.

16. The Compound 1 Na salt Form A according to any one of Embodiments 10-15, characterized by an X-ray powder diffractogram substantially similar to FIG. 2A.

17. A method of making the Compound 1 Na salt Form A according to any one of Embodiments 10-16, comprising:
(a) reacting Compound 1 Form A with NaOH in the presence of acetone to form a second reaction mixture; and
(b) isolating a solid portion from step (a) and drying the solid portion to yield Compound 1 Na salt Form A.

18. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Na salt Form B.

19. The Compound 1 Na salt Form B according to Embodiment 18, characterized by an X-ray powder diffractogram having signals at 3.1±0.2 degrees two-theta and 8.9±0.2 degrees two-theta.

20. The Compound 1 Na salt Form B according to Embodiment 18 or 19, characterized by an X-ray powder diffractogram having signals at 3.1±0.2 degrees two-theta, 8.9±0.2 degrees two-theta, 17.8±0.2 degrees two-theta, and 26.9±0.2 degrees two-theta.

21. The Compound 1 Na salt Form B according to any one of Embodiments 18-20, characterized by an X-ray powder diffractogram substantially similar to FIG. 3A.

22. A method of making the Compound 1 Na salt Form B according to any one of Embodiments 18-21, comprising:
(a) reacting Compound 1 Form A with NaOH in the presence of ethyl acetate to form a second reaction mixture; and
(b) isolating a solid portion from step (a) and drying the solid portion to yield Compound 1 Na salt Form B.

23. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Na salt Form C.

24. The Compound 1 Na salt Form C according to Embodiment 23, characterized by an X-ray powder diffractogram having signals at 19.7±0.2 degrees two-theta, 9.2±0.2 degrees two-theta, and 13.3±0.2 degrees two-theta.

25. The Compound 1 Na salt Form C according to Embodiment 23 or 24, characterized by an X-ray powder diffractogram having (a) signals at 19.7±0.2 degrees two-theta, 9.2-10.2 degrees two-theta, and 13.3±0.2 degrees two-theta; and (b) at least one signal selected from 10.4±0.2 degrees two-theta, 11.9±0.2 degrees two-theta, 17.1±0.2 degrees two-theta, 17.7±0.2 degrees two-theta, 20.7±0.2 degrees two-theta, 19.2±0.2 degrees two-theta, 20.8±0.2 degrees two-theta, 23.9±0.2 degrees two-theta, 26.6±0.2 degrees two-theta, 26.7±0.2 degrees two-theta, and 27.2±0.2 degrees two-theta.

26. The Compound 1 Na salt Form C according to any one of Embodiments 23-25, characterized by an X-ray powder diffractogram substantially similar to FIG. 4A.

27. The Compound 1 Na salt Form C according to any one of Embodiments 23-26, characterized by a ¹³C ssNMR peak at one or more of 138.1±0.2 ppm, 121.5±0.2 ppm, 117.4±0.2 ppm, 115.2±0.2 ppm, 36.7±0.2 ppm, and 32.1±0.2 ppm.

28. The Compound 1 Na salt Form C according to any one of Embodiments 23-27, characterized by a ¹³C ssNMR spectrum substantially similar to FIG. 4B.

29. The Compound 1 Na salt Form C according to any one of Embodiments 23-28, characterized by a ²³Na ssNMR peak at −11.2±0.2 ppm and/or −14.0±0.2 ppm.

30. The Compound 1 Na salt Form C according to any one of Embodiments 23-29, characterized by a ²Na ssNMR spectrum substantially similar to FIG. 4C.

31. A method of making the Compound 1 Na salt Form C according to any one of Embodiments 23-30, comprising:
(a) reacting Compound 1 Form A with NaOH in the presence of a polyethylene glycol to form a second reaction mixture; and
(b) isolating a solid portion from step (a) to yield Compound 1 Na salt Form C.

32. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Na salt Form D.

33. The Compound 1 Na salt Form D according to Embodiment 32, characterized by an X-ray powder diffractogram having signals 3.5±0.2 degrees two-theta and 16.2±0.2 degrees two-theta.

34. The Compound 1 Na salt Form D according to Embodiment 32 or 33, characterized by an X-ray powder diffractogram having signals at 3.5±0.2 degrees two-theta and 16.2±0.2 degrees two-theta and at least one of 18.7±0.2 degrees two-theta and 17.5±0.2 degrees two-theta.

35. The Compound 1 Na salt Form D according to any one of Embodiments 32-34, characterized by an X-ray powder diffractogram having signals at 3.5±0.2 degrees two-theta, 16.2±0.2 degrees two-theta, 18.7±0.2 degrees two-theta, and 17.5±0.2 degrees two-theta.

36. The Compound 1 Na salt Form D according to any one of Embodiments 32-35, characterized by an X-ray powder diffractogram having (a) signals at 3.5±0.2 degrees two-theta, 16.2±0.2 degrees two-theta, 18.7±0.2 degrees two-theta, and 17.5±0.2 degrees two-theta; and (b) at least one signal selected from 13.7±0.2 degrees two-theta, 14.0±0.2 degrees two-theta, 17.2±0.2 degrees two-theta, 19.3±0.2 degrees two-theta, 20.0±0.2 degrees two-theta, 21.3±0.2 degrees two-theta, 21.8±0.2 degrees two-theta, 22.7±0.2 degrees two-theta, 28.8±0.2 degrees two-theta, and 30.9±0.2 degrees two-theta.

37. The Compound 1 Na salt Form D according to any one of Embodiments 32-36, characterized by an X-ray powder diffractogram substantially similar to FIG. 5A.

38. The Compound 1 Na salt Form D according to any one of Embodiments 32-37, characterized by a ¹³C ssNMR peak at one or more of 175.8±0.2 ppm, 142.0±0.2 ppm, 134.0±0.2 ppm, 119.3±0.2 ppm, 97.9±0.2 ppm, 67.7±0.2 ppm, and 37.2±0.2 ppm.

39. The Compound 1 Na salt Form D according to any one of Embodiments 32-37, characterized by a ¹³C ssNMR spectrum substantially similar to FIG. 5B.

40. The Compound 1 Na salt Form D according to any one of Embodiments 32-39, characterized by a ²³Na ssNMR peak at one or more of 5.3±0.2 ppm, 2.1±0.2 ppm, −5.0±0.2 ppm, and −6.3±0.2 ppm.

41. The Compound 1 Na salt Form D according to any one of Embodiments 32-40, characterized by a ²³Na ssNMR spectrum substantially similar to FIG. 5C.

42. A method of making the Compound 1 Na salt Form D according to any one of Embodiments 32-41, comprising:
(a) reacting Compound 1 Form A with NaOH at 4-10° C. to form a second reaction mixture; and
(b) isolating a solid portion from step (a) to yield Compound 1 Na salt Form D.

43. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Ca salt Form A.

44. The Compound 1 Ca salt Form A according to Embodiment 43, characterized by an X-ray powder diffractogram having signals at 17.9±0.2 degrees two-theta and at least one of 11.7±0.2 degrees two-theta and 20.5±0.2 degrees two-theta.

45. The Compound 1 Ca salt Form A according to Embodiment 43 or 44, characterized by an X-ray powder diffractogram having signals at 17.9±0.2 degrees two-theta, 11.7±0.2 degrees two-theta, and 20.5±0.2 degrees two-theta.

46. The Compound 1 Ca salt Form A according to any one of Embodiments 43-45, characterized by an X-ray powder diffractogram having (a) signals at 17.9±0.2 degrees two-theta, 11.7±0.2 degrees two-theta, and 20.5±0.2 degrees two-theta; and (b) at least one signal selected from 5.2±0.2 degrees two-theta, 7.3±0.2 degrees two-theta, 9.9±0.2 degrees two-theta, 10.6±0.2 degrees two-theta, 12.4±0.2 degrees two-theta, 14.5±0.2 degrees two-theta, 16.4±0.2 degrees two-theta, 18.6±0.2 degrees two-theta, 19.2±0.2 degrees two-theta, 20.9±0.2 degrees two-theta, 22.0±0.2 degrees two-theta, 23.5±0.2 degrees two-theta, 24.1±0.2 degrees two-theta, and 24.7±0.2 degrees two-theta.

47. The Compound 1 Ca salt Form A according to any one of Embodiments 43-46, characterized by an X-ray powder diffractogram substantially similar to FIG. 6A.

48. A method of making the Compound 1 Ca salt Form A according to any one of Embodiments 43-47, comprising:
(a) reacting Compound 1 Form A with Ca(OH)₂ in the presence of a second organic solvent to form a second reaction mixture; and
(b) isolating a solid portion from step (a) and drying the solid portion to yield Compound 1 Ca salt Form A.

49. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) HCl salt Form A.

50. The Compound 1 HCl salt Form A according to Embodiment 49, characterized by an X-ray powder diffractogram having a signal at one or more of 8.1±0.2 degrees two-theta, 7.8±0.2 degrees two-theta, and 9.0±0.2 degrees two-theta.

51. The Compound 1 HCl salt Form A according to Embodiment 49 or 50, characterized by an X-ray powder diffractogram having signals at 8.1±0.2 degrees two-theta, 7.8±0.2 degrees two-theta, and 9.0±0.2 degrees two-theta.

52. The Compound 1 HCl salt Form A according to any one of Embodiments 49-51, characterized by an X-ray powder diffractogram having (a) signals at 8.1±0.2 degrees two-theta, 7.8±0.2 degrees two-theta, and 9.0±0.2 degrees two-theta; and (b) at least one signal selected from 19.8±0.2 degrees two-theta, 20.1±0.2 degrees two-theta, and 23.8±0.2 degrees two-theta.

53. The Compound 1 HCl salt Form A according to any one of Embodiments 49-52, characterized by an X-ray powder diffractogram substantially similar to FIG. 7A.

54. A method of making the Compound 1 HCl salt Form A according to any one of Embodiments 49-53, comprising:
(a) reacting Compound 1 Form A with HCl in the presence of a second organic solvent via slurry to form a second reaction mixture; and
(b) isolating a solid portion from step (a) and drying the solid portion to yield Compound 1 HCl salt Form A.

55. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) DMSO solvate Form A.

56. The Compound 1 DMSO solvate Form A according to Embodiment 55, characterized by an X-ray powder diffractogram having a signal at one or more of 9.9±0.2 degrees two-theta, 19.1±0.2 degrees two-theta, and 19.8±0.2 degrees two-theta.

57. The Compound 1 DMSO solvate Form A according to Embodiment 55 or 56, characterized by an X-ray powder diffractogram having signals at 9.9±0.2 degrees two-theta, 19.1±0.2 degrees two-theta, and 19.8±0.2 degrees two-theta.

58. The Compound 1 DMSO solvate Form A according to any one of Embodiments 55-57, characterized by an X-ray powder diffractogram having (a) signals at 9.9±0.2 degrees two-theta, 19.1±0.2 degrees two-theta, and 19.8±0.2 degrees two-theta; and (b) at least one signal selected from 4.9±0.2 degrees two-theta, 7.1±0.2 degrees two-theta, 11.0±0.2 degrees two-theta, 14.8±0.2 degrees two-theta, and 20.7±0.2 degrees two-theta.

59. The Compound 1 DMSO solvate Form A according to any one of Embodiments 55-58, characterized by an X-ray powder diffractogram substantially similar to FIG. 8A.

60. A method of making the Compound 1 DMSO solvate Form A according to any one of Embodiments 55-59, comprising:
(a) reacting Compound 1 Form A with DMSO to form a second reaction mixture at 90-110° C.; and
(b) isolating a solid portion from step (a) and drying the solid portion to yield Compound 1 DMSO solvate Form A.

61. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) EtOH solvate Form A.

62. The Compound 1 EtOH solvate Form A according to Embodiment 61, characterized by an X-ray powder diffractogram having a signal at one or more of 20.2±0.2 degrees two-theta, 20.7±0.2 degrees two-theta, and 23.4±0.2 degrees two-theta.

63. The Compound 1 EtOH solvate Form A according to Embodiment 61 or 62, characterized by an X-ray powder diffractogram having signals at 20.2±0.2 degrees two-theta, 20.7±0.2 degrees two-theta, and 23.4±0.2 degrees two-theta.

64. The Compound 1 EtOH solvate Form A according to any one of Embodiments 61-63, characterized by an X-ray powder diffractogram having (a) signals at 20.2±0.2 degrees two-theta, 20.7±0.2 degrees two-theta, and 23.4±0.2 degrees two-theta; and (b) at least one signal selected from 7.5±0.2 degrees two-theta, 12.0±0.2 degrees two-theta, 12.6±0.2 degrees two-theta, 13.8±0.2 degrees two-theta, 15.9±0.2 degrees two-theta, 16.6±0.2 degrees two-theta, 17.1±0.2 degrees two-theta, 18.2±0.2 degrees two-theta, 18.9±0.2 degrees two-theta, 19.8±0.2 degrees two-theta, 21.0±0.2 degrees two-theta, 21.4±0.2 degrees two-theta, 22.4±0.2 degrees two-theta, 22.9±0.2 degrees two-theta, 24.6±0.2 degrees two-theta, 26.4±0.2 degrees two-theta, 26.7±0.2 degrees two-theta, 28.6±0.2 degrees two-theta, 29.2±0.2 degrees two-theta, and 29.6±0.2 degrees two-theta.

65. The Compound 1 EtOH solvate Form A according to any one of Embodiments 61-64, characterized by an X-ray powder diffractogram substantially similar to FIG. 9A.

66. The Compound 1 EtOH solvate Form A according to any one of Embodiments 61-65, characterized by a ¹³C ssNMR peak at one or more of 126.6±0.2 ppm, 111.5±0.2 ppm, 57.9±0.2 ppm, 34.4±0.2 ppm, 27.9±0.2 ppm, and 19.0±0.2 ppm.

67. The Compound 1 EtOH solvate Form A according to any one of Embodiments 61-65, characterized by a ¹³C ssNMR spectrum substantially similar to FIG. 9B.

68. A method of making the Compound 1 EtOH solvate Form A according to any one of Embodiments 61-67, comprising:
(a) dissolving Compound 1 in an organic solvent to form a first reaction mixture at 55-65° C.;
(b) adding water to the reaction mixture to precipitate a first solid portion;
(c) isolating the first solid portion and re-suspending the first solid portion in EtOH to form a second reaction mixture; and
(d) isolating a second solid portion from the second reaction mixture and drying the second solid portion to yield Compound 1 EtOH solvate Form A.

69. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) tartrate salt or cocrystal Form A.

70. The Compound 1 tartrate salt or cocrystal Form A according to Embodiment 69, characterized by an X-ray powder diffractogram having signals at 19.0±0.2 degrees two-theta, 19.6±0.2 degrees two-theta, and 20.5±0.2 degrees two-theta.

71. The Compound 1 tartrate salt or cocrystal Form A according to Embodiment 69 or 70, characterized by an X-ray powder diffractogram having (a) signals at 19.0±0.2 degrees two-theta, 19.6±0.2 degrees two-theta, and 20.5±0.2 degrees two-theta; and (b) at least one signal selected from 19.4±0.2 degrees two-theta, 22.1±0.2 degrees two-theta, 26.5±0.2 degrees two-theta, and 26.6±0.2 degrees two-theta.

72. The Compound 1 tartrate salt or cocrystal Form A according to any one of Embodiments 69-71, characterized by an X-ray powder diffractogram substantially similar to FIG. 10A.

73. A method of making the Compound 1 tartrate salt or cocrystal Form A according to any one of Embodiments 69-72, comprising:
(a) reacting Compound 1 Form A with tartaric acid in the presence of a second base and THF/water to form a second reaction mixture; and
(b) evaporating a solid portion from the second reaction mixture to yield Compound 1 tartrate salt or cocrystal Form A.

74. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) tartrate salt or cocrystal Form B.

75. The Compound 1 tartrate salt or cocrystal Form B according to Embodiment 74, characterized by an X-ray powder diffractogram having signals at 8.9±0.2 degrees two-theta, 17.8±0.2 degrees two-theta, and 22.7±0.2 degrees two-theta.

76. The Compound 1 tartrate salt or cocrystal Form B according to Embodiment 74 or 75, characterized by an X-ray powder diffractogram having (a) signals at 8.9±0.2 degrees two-theta, 17.8±0.2 degrees two-theta, and 22.7±0.2 degrees two-theta; and (b) at least one signal selected from 6.6±0.2 degrees two-theta, 11.9±0.2 degrees two-theta, 12.9±0.2 degrees two-theta, 16.8±0.2 degrees two-theta, 18.2±0.2 degrees two-theta, 18.8±0.2 degrees two-theta, 19.3±0.2 degrees two-theta, 19.8±0.2 degrees two-theta, 20.1±0.2 degrees two-theta, 20.3±0.2 degrees two-theta, 20.8±0.2 degrees two-theta, 21.7±0.2 degrees two-theta, 22.0±0.2 degrees two-theta, 22.3±0.2 degrees two-theta, 24.7±0.2 degrees two-theta, 26.0±0.2 degrees two-theta, 26.5±0.2 degrees two-theta, 23.6±0.2 degrees two-theta, and 29.5±0.2 degrees two-theta.

77. The Compound 1 tartrate salt or cocrystal Form B according to any one of Embodiments 74-76, characterized by an X-ray powder diffractogram substantially similar to FIG. 11A.

78. A method of making the Compound 1 tartrate salt or cocrystal Form B according to any one of Embodiments 74-77, comprising:
(a) reacting Compound 1 Form A with tartaric acid in the presence of a base and ethyl acetate to form a second reaction mixture; and
(b) evaporating a solid portion from the second reaction mixture to yield Compound 1 tartrate salt or cocrystal Form B.

79. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) tartrate salt or cocrystal Form C.

80. The Compound 1 tartrate salt or cocrystal Form C according to Embodiment 79, characterized by an X-ray powder diffractogram having signals at 12.4±0.2 degrees two-theta, 13.3±0.2 degrees two-theta, and 18.5±0.2 degrees two-theta.

81. The Compound 1 tartrate salt or cocrystal Form C according Embodiment 79 or 80, characterized by an X-ray powder diffractogram having (a) signals at 12.4±0.2 degrees two-theta, 13.3±0.2 degrees two-theta, and 18.5±0.2 degrees two-theta; and (b) at least one signal selected from 15.8±0.2 degrees two-theta, 16.8±0.2 degrees two-theta, 19.4±0.2 degrees two-theta, 21.5±0.2 degrees two-theta, 22.5±0.2 degrees two-theta, 27.1±0.2 degrees two-theta, 29.2±0.2 degrees two-theta, and 29.5±0.2 degrees two-theta.

82. The Compound 1 tartrate salt or cocrystal Form C according to any one of Embodiments 79-81, characterized by an X-ray powder diffractogram substantially similar to FIG. 12A.

83. A method of making the Compound 1 tartrate salt or cocrystal Form C according to any one of Embodiments 79-82, comprising:
(a) reacting Compound 1 Form A with tartaric acid in the presence of Ca(OH)₂ and THF to form a second reaction mixture; and
(b) evaporating a solid portion from the second reaction mixture to yield Compound 1 tartrate salt or cocrystal Form C.

84. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) tartrate salt or cocrystal Form D.

85. The Compound 1 tartrate salt or cocrystal Form D according to Embodiment 84, characterized by an X-ray powder diffractogram having a signal at one or more of 13.8±0.2 degrees two-theta, 14.8±0.2 degrees two-theta, and 25.2±0.2 degrees two-theta.

86. The Compound 1 tartrate salt or cocrystal Form D according to Embodiment 84 or 85, characterized by an X-ray powder diffractogram having signals at 13.8±0.2 degrees two-theta, 14.8±0.2 degrees two-theta, and 25.2±0.2 degrees two-theta.

87. The Compound 1 tartrate salt or cocrystal Form D according to any one of Embodiments 84-86, characterized by an X-ray powder diffractogram having (a) signals at 13.8±0.2 degrees two-theta, 14.8±0.2 degrees two-theta, and 25.2±0.2 degrees two-theta; and (b) at least one signal selected from 12.5±0.2 degrees two-theta, 18.7±0.2 degrees two-theta, 19.5±0.2 degrees two-theta, 21.9±0.2 degrees two-theta, 22.5±0.2 degrees two-theta, 23.9±0.2 degrees two-theta, 24.5±0.2 degrees two-theta, 27.7±0.2 degrees two-theta, and 28.3±0.2 degrees two-theta.

88. The Compound 1 tartrate salt or cocrystal Form D according to any one of Embodiments 84-87, characterized by an X-ray powder diffractogram substantially similar to FIG. 13.

89. A method of making the Compound 1 tartrate salt or cocrystal Form D according to any one of Embodiments 84-88, comprising:
(a) reacting Compound 1 Form A with tartaric acid in the presence of Mg(OH)₂ and THE to form a second reaction mixture; and
(b) evaporating a solid portion from the second reaction mixture to yield Compound 1 tartrate salt or cocrystal Form D.

90. The method according to any one of Embodiments 17, 22, 31, 42, 48, 54, 60, 73, 78, 83, and 89, wherein the Compound 1 Form A is prepared using a method comprising the steps of:
(i) contacting methyl 4-(5-(4-fluorophenyl)-1-pivaloyl-6-(tetrahydro-2H-pyran-4-yl)-1,5-dihydropyrrolo[2,3-f]indazol-7-yl)benzoate with a first organic solvent and a first base to form a first reaction mixture;
(ii) adding water and a first acid to the first reaction mixture;
(iii) isolating an organic portion from step (ii), adding an alcohol and optionally adding water to the organic portion, and concentrating the mixture by distillation; and
(iv) isolating Compound 1 from the mixture from step (iii) and drying the material to remove all water content to yield Compound 1 Form A.

91. A solid dispersion comprising a solid form of 4-(5-(4-fluorophenyl)-6-(tetrahydro-2H-pyran-4-yl)-1,5-dihydropyrrolo[2,3-f]indazol-7-yl)benzoic acid (Compound 1) or a salt, solvate, or cocrystal thereof and a polymer carrier; wherein the solid dispersion is prepared by dissolving the solid form of Compound 1 or a salt, solvate, or cocrystal thereof in a solvent system comprising a first organic solvent, a second organic solvent, and optionally water; wherein:
when water is absent from the solvent system, the volume ratio of the first organic solvent to the second organic solvent is between about 55/45 v/v and about 90/10 v/v; and
when water is present in the solvent system, the weight ratio of the first organic solvent to the second organic solvent and to water is between about 55/35/10 w/w and about 80/10/10; wherein when the weight ratio of the first organic solvent to the second organic solvent and to water is about 55/35/10 w/w, the solid dispersion comprises greater than about 50% w/w of the solid form of Compound 1 or a salt, solvate, or cocrystal thereof.

92. The solid dispersion according to Embodiment 91, wherein the solid dispersion comprises no lower than about 50% w/w of the solid form of Compound 1 or a salt, solvate, or cocrystal thereof; and wherein when the weight ratio of the first organic solvent to the second organic solvent and to water is about 55/35/10 w/w, the solid dispersion comprises higher than about 50% w/w of the solid form of Compound 1 or a salt, solvate, or cocrystal thereof.

93. The solid dispersion according to Embodiment 91 or 92, wherein the polymer is PVP-VA or HPMCAS-H.

94. The solid dispersion according to any one of Embodiments 91-93, wherein the first organic solvent is selected from DCM, THF, and Me-THF.

95. The solid dispersion according to any one of Embodiments 91-94, wherein the second organic solvent is MeOH or EtOH.

96. The solid dispersion according to any one of Embodiments 91-95, wherein the solid dispersion is a spray dried dispersion.

97. A method of preparing a solid dispersion comprising a solid form of 4-(5-(4-fluorophenyl)-6-(tetrahydro-2H-pyran-4-yl)-1,5-dihydropyrrolo[2,3-f]indazol-7-yl)benzoic acid (Compound 1) or a pharmaceutically acceptable salt thereof and a polymer carrier, the method comprising:
(a) dissolving the solid form of Compound 1 or a salt, solvate, or cocrystal thereof in a solvent system comprising a first organic solvent, a second organic solvent, and optionally water to form a reaction mixture;
(b) adding the polymer carrier to the reaction mixture and stirring the reaction mixture at ambient temperature; and
(c) spray drying the reaction mixture to yield the solid dispersion as a final product;
wherein:
when water is absent from the solvent system, the volume ratio of the first organic solvent to the second organic solvent is between about 55/45 v/v and about 90/10 v/v; and
when water is present in the solvent system, the weight ratio of the first organic solvent to the second organic solvent and to water is between about 55/35/10 w/w and about 80/10/10; wherein when the weight ratio of the first organic solvent to the second organic solvent and to water is about 55/35/10 w/w, the solid dispersion as a final product comprises greater than about 50% w/w of the solid form of Compound or a salt, solvate, or cocrystal thereof.

98. A compound represented by one of the following structural formulae:

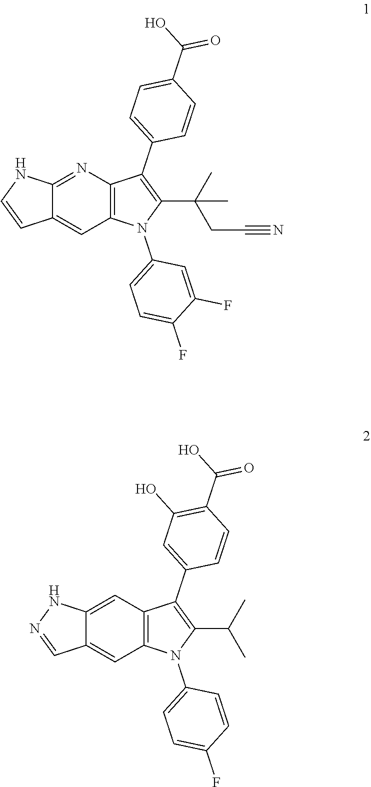 [SMILES: CC(C)(CC#N)c1c(-c2ccc(C(=O)O)cc2)c2nc3[nH]ccc3cc2n1-c1ccc(F)c(F)c1 ; CC(C)c1c(-c2ccc(C(=O)O)c(O)c2)c2cc3[nH]ncc3cc2n1-c1ccc(F)cc1]

a tautomer thereof, a deuterated derivative of that compound or tautomer, or a pharmaceutically acceptable salt of the foregoing.

99. A pharmaceutical composition comprising the Compound 1 neat Form C according to any one of Embodiments 1-8; or the Compound 1 Na salt Form A according to any one of Embodiments 10-16; or the Compound 1 Na salt Form B according to any one of Embodiments 18-21; or the Compound 1 Na salt Form C according to any one of Embodiments 23-30; or the Compound 1 Na salt Form D according to any one of Embodiments 32-41; or the Compound 1 Ca salt Form A according to any one of Embodiments 43-47; or the Compound HCl salt Form A according to any one of Embodiments 49-53; or the Compound 1 DMSO solvate Form A according to any one of Embodiments 55-59; or the Compound 1 EtOH solvate Form A according to any one of Embodiments 61-67; or the Compound 1 tartrate salt or cocrystal Form A according to any one of Embodiments 69-72; or the Compound 1 tartrate salt or cocrystal Form B according to any one of Embodiments 74-77; or the Compound 1 tartrate salt or cocrystal Form C according to any one of Embodiments 79-82; or the Compound 1 tartrate salt or cocrystal Form D according to any one of Embodiments 84-88; or the solid dispersion according to any one of Embodiments 91-96; or the compound according to Embodiment 98 or a tautomer thereof, a deuterated derivative of that compound or tautomer, or a pharmaceutically acceptable salt of the foregoing; and a pharmaceutically acceptable carrier.

100. A method of treating alpha-1 antitrypsin deficiency comprising administering to a patient in need thereof the Compound 1 neat Form C according to any one of Embodiments 1-8; or the Compound 1 Na salt Form A according to any one of Embodiments 10-16; or the Compound 1 Na salt Form B according to any one of Embodiments 18-21; or the Compound 1 Na Salt Form C according to any one of Embodiments 23-30; or the Compound 1 Na salt Form D according to any one of Embodiments 32-41; or the Compound 1 Ca salt Form A according to any one of Embodiments 43-47; or the Compound HCl salt Form A according to any one of Embodiments 49-53; or the Compound 1 DMSO solvate Form A according to any one of Embodiments 55-59; or the Compound 1 EtOH solvate Form A according to any one of Embodiments 61-67; or the Compound 1 tartrate salt or cocrystal Form A according to any one of Embodiments 69-72; or the Compound 1 tartrate salt or cocrystal Form B according to any one of Embodiments 74-77; or the Compound 1 tartrate salt or cocrystal Form C according to any one of Embodiments 79-82; or the Compound 1 tartrate salt or cocrystal Form D according to any one of Embodiments 84-88; or the solid dispersion according to any one of Embodiments 91-96; or the compound according to Embodiment 98 or a tautomer thereof, a deuterated derivative of that compound or tautomer, or a pharmaceutically acceptable salt of the foregoing; or the pharmaceutical composition according to Embodiment 99.

101. The method according to Embodiment 100, wherein the patient has a Z mutation in alpha-1 antitrypsin.

102. The method according to Embodiment 100, wherein the patient has an SZ mutation in alpha-1 antitrypsin.

103. The method according to Embodiment 100, wherein the patient is homozygous for Z-mutations in alpha-1 antitrypsin.

104. A method of modulating alpha-1 antitrypsin activity comprising contacting said alpha-1-antitrypsin with the Compound 1 neat Form C according to any one of Embodiments 1-8; or the Compound 1 Na salt Form A according to any one of Embodiments 10-16; or the Compound 1 Na salt Form B according to any one of Embodiments 18-21; or the Compound 1 Na Salt Form C according to any one of Embodiments 23-30; or the Compound 1 Na salt Form D according to any one of Embodiments 32-41; or the Compound 1 Ca salt Form A according to any one of Embodiments 43-47; or the Compound HCl salt Form A according to any one of Embodiments 49-53; or the Compound 1 DMSO solvate Form A according to any one of Embodiments 55-59; or the Compound 1 EtOH solvate Form A according to any one of Embodiments 61-67; or the Compound 1 tartrate salt or cocrystal Form A according to any one of Embodiments 69-72; or the Compound 1 tartrate salt or cocrystal Form B according to any one of Embodiments 74-77; or the Compound 1 tartrate salt or cocrystal Form C according to any one of Embodiments 79-82; or the Compound 1 tartrate salt or cocrystal Form D according to any one of Embodiments 84-88; or the solid dispersion according to any one of Embodiments 91-96; or the compound according to Embodiment 98 or a tautomer thereof, a deuterated derivative of that compound or tautomer, or a pharmaceutically acceptable salt of the foregoing; or the pharmaceutical composition according to Embodiment 99.

### EXAMPLES

In order that the disclosure described herein may be more fully understood, the following examples are set forth. It should be understood that these examples are for illustrative purposes only and are not to be construed as limiting this disclosure in any manner.

### Example 1. Preparation of Compound 1 and Compound 1 Form A

### Preparation of Compound 1

Compound 1 may be made according to standard chemical practices or as described herein. Throughout the following synthetic schemes and in the descriptions for preparing the solid forms of Compound 1, the following abbreviations are used:

### Abbreviations

18-crown-6=1,4,7,10,13,16-hexaoxacyclooctadecane
BrettPhos Pd G1=chloro[2-(dicyclohexylphosphino)-3,6-dimethoxy-2′,4′, 6′-triisopropyl-1,1′-biphenyl][2-(2-aminoethyl)phenyl]palladium(II) or (BrettPhos) palladium(II) phenethylamine chloride
BrettPhos Pd G4=dicyclohexyl-[3,6-dimethoxy-2-[2,4,6-tri(propan-2-yl)phenyl]phenyl]phosphane;methanesulfonic acid;N-methyl-2-phenylaniline;palladium
CBzCl=benzyl chloroformate
Cphos=2-dicyclohexylphosphino-2′,6′-bis(N,N-dimethylamino)biphenyl
Cs₂CO₃=cesium carbonate
DCE=1,2-dichloroethane
DIPEA=N,N-diisopropylethylamine or N-ethyl-N-isopropyl-propan-2-amine
DMAP=dimethylamino pyridine
DMF=dimethylformamide
DMSO=dimethyl sulfoxide
Dppf=1,1′-ferrocenediyl-bis(diphenylphosphine)
DTBPF=1,1′-bis(di-tert-butylphosphino)ferrocene
EtOAc=ethyl acetate
HATU=[dimethylamino(triazolo[4,5-b]pyridin-3-yloxy)methylene]-dimethyl-ammonium (Phosphorus Hexafluoride Ion)
IPA=isopropyl alcohol
KOtBu=potassium tert-butoxide
K₃PO₄=potassium phosphate tribasic
MeOH=methanol
MP-TMT scavenger resin=a macroporous polystyrene-bound trimercaptotriazine, a resin bound equivalent of 2,4,6-trimercaptotriazine (TMT).
MTBE=methyl tert-butyl ether
NaCNBH₃=sodium cyanoborohydride
NMM=N-methyl morpholine
NaOtBu=sodium tert-butoxide
Pd₂(dba)₃=tris(dibenzylideneacetone)dipalladium (0)
Pd(dppf)₂Cl₂=[1,1′-Bis(diphenylphosphino)ferrocene]dichloropalladium(II)
PdCl₂(PPh₃)₂=bis(triphenylphosphine)palladium(II) dichloride
Pd(OAc)₂=palladium(II) acetate
Pd(tBu₃P)₂=bis(tri-tert-butylphosphine)palladium(0)
PivCl=pivaloyl chloride
PTSA=p-toluenesulfonic acid monohydrate
rac-BINAP=(±)-2,2′-bis(diphenylphosphino)-1,1′-binaphthalene
[Rh(COD)Cl]2=chloro(1,5-cyclooctadiene)rhodium (I) dimer
SEMCI=2-(trimethylsilyl)ethoxymethyl chloride
SFC=super critical fluid chromatography
SPhos=2-dicyclohexylphosphino-2′,6′-dimethoxybiphenyl
SPhos Pd G4=dicyclohexyl-[2-(2,6-dimethoxyphenyl)phenyl]phosphane;methanesulfonic acid;N-methyl-2-phenylaniline;palladium
SPM32=3-mercaptopropyl ethyl sulfide Silica
TBAB=tetrabutylammonium bromide
TBAF=tetrabutylammonium fluoride
tBuXPhos Pd G1=chloro[2-(di-tert-butylphosphino)-2′,4′,6′-triisopropyl-1,1′-biphenyl][2-(2-aminoethyl)phenyl)]palladium(II) or t-BuXPhos palladium(II) phenethylamine chloride
tBuXPhos Pd G3=[(2-di-tert-butylphosphino-2′,4′,6′-triisopropyl-1,1′-biphenyl)-2-(2′-amino-1,1′-biphenyl)] palladium(II) methanesulfonate
tBuXPhos Pd G4=methanesulfonato(2-di-t-butylphosphino-2′,4′,6′-tri-i-propyl-1,1′-biphenyl)(2′-methylamino-1,1′-biphenyl-2-yl)palladium(II) dichloromethane
TEA=triethylamine
TFA=trifluoroacetic acid
THE=tetrahydrofuran
THP=tetrahydropyran
TMSI=iodotrimethylsilane
XantPhos Pd G3=[(4,5-bis(diphenylphosphino)-9,9-dimethylxanthene)-2-(2′-amino-1,1′-biphenyl)]palladium(II) methanesulfonate
XPhos Pd G1=(2-dicyclohexylphosphino-2′,4′,6′-triisopropyl-1,1′-biphenyl)[2-(2-aminoethyl)phenyl)]palladium(II) chloride or (XPhos) palladium(II) phenethylamine chloride
XPhos Pd G3=(2-dicyclohexylphosphino-2′,4′,6′-triisopropyl-1,1′-biphenyl)[2-(2′-amino-1,1′-biphenyl)]palladium(II) methanesulfonate

In some embodiments, processes for preparing Compound 1 comprise reactions depicted in Schemes 1-3 below:

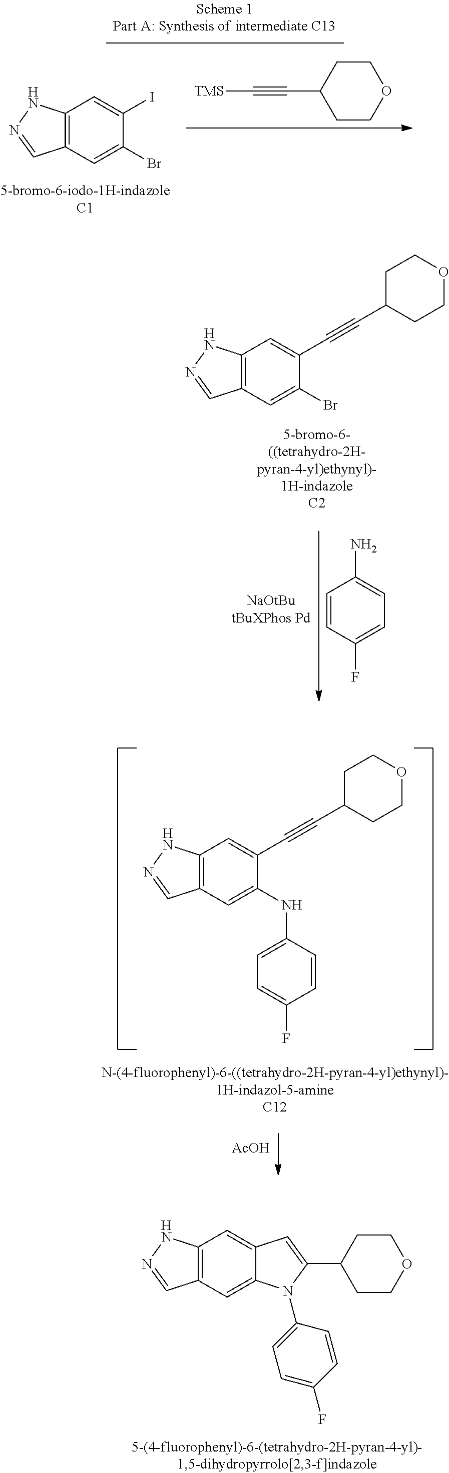

### Step 1. Synthesis of 5-bromo-6-(2-tetrahydropyran-4-ylethynyl)-1H-indazole (C2)

To a solution of 5-bromo-6-iodo-1H-indazole C1 (100 g, 294.2 mmol) in 1,4-dioxane (500 mL) was added Et₃N (500 mL, 3.6 mol), copper iodide (3.4 g, 17.9 mmol), CsF (89.4 g, 588.5 mmol), H₂O (10.6 mL, 588.4 mmol), and Pd(PPh₃)₂Cl₂ (6.2 g, 8.8 mmol). Trimethyl((tetrahydro-2H-pyran-4-yl)ethynyl)silane (67 g, 367.5 mmol) was added, and the reaction mixture was purged with nitrogen for 15 min, then heated to 80° C. overnight. Upon cooling, Et₃N and 1,4-dioxane were removed by concentration in vacuo. Water (200 mL) and brine (200 mL) were added and the mixture was extracted with EtOAc (1.4 L). The combined organic layers were dried and concentration in vacuo. Ethyl acetate (120 mL) was added, and the mixture was stirred for 1 h. The resulting solid which formed was filtered, and washed with EtOAc (×2) to afford the desired product as a solid (43 g). The filtrate was concentrated and purified by silica gel chromatography (Column: 800 g Silica Gel. Eluent: 25% CH₂Cl₂ in heptane, followed by a gradient of 0-90% CH₂Cl₂ in heptane) to afford additional product as a brown solid (29 g). The product batches were combined to afford the product as a brown solid (72 g, 80%). ¹H NMR (300 MHz, Chloroform-d) δ 10.43 (s, 1H), 8.00 (dd, J=3.0, 0.9 Hz, 2H), 7.62 (t, J=0.8 Hz, 1H), 4.02 (ddd, J=11.6, 6.5, 3.5 Hz, 2H), 3.62 (ddd, J=11.3, 7.7, 3.2 Hz, 2H), 2.98 (tt, J=8.0, 4.2 Hz, 1H), 2.02-1.89 (m, 2H), 1.82 (dtd, J=13.4, 7.7, 3.5 Hz, 2H). LCMS m/z 306.8 [M+H]⁺.

### Steps 2 & 3. Synthesis of 5-(4-fluorophenyl)-6-tetrahydropyran-4-yl-1H-pyrrolo[2,3-f]indazole (C13)

A mixture of 5-bromo-6-(2-tetrahydropyran-4-ylethynyl)-1H-indazole C2 (160 g, 524.3 mmol), 4-fluoroaniline (75 mL, 791.7 mmol), NaOtBu (90 g, 936.5 mmol) in tBuOH (2.1 L) at 40° C. was purged with nitrogen for 10 min. tBuXPhos Pd G1 (10.8 g, 15.7 mmol) was added, and the mixture purged with nitrogen for an additional 10 min. The mixture was heated to 80° C. for 1 h, and then concentrated in vacuo. CH₂Cl₂ (1.5 L), saturated NH₄Cl (1 L), and HCl (62 mL of 6 M, 372.0 mmol) were added. The organic layer was dried with Na₂SO₄, concentrated in vacuo, and re-dissolved in CH₂Cl₂ (160 mL). The mixture was filtered to remove the white inorganic solid. The filtrate was then purified by silica chromatography (Column: 3 kg Silica gel. Gradient: 0-90% EtOAc in heptane) to afford the product contaminated with 4-fluoroaniline. The mixture was dissolved in EtOAc (1.5 L), and washed with 1N HCl (2×250 mL), then brine. The organic layer was dried, and concentrated in vacuo to afford the product as a sticky solid, which was used without further purification (160 g, 91%). LCMS m/z 336.1 [M+H]⁺.

A solution of N-(4-fluorophenyl)-6-(2-tetrahydropyran-4-ylethynyl)-1H-indazol-5-amine C12 in DMSO (550 mL) was heated to 160° C. for 1.5 h. The mixture was cooled, and sat. Na₂CO₃ (500 mL) and water (1.5 L) were added. The mixture was allowed to stir overnight. The resulting grey solid suspension was filtered, and the filter cake was washed with water (×3), then heptane (×3). The filter cake was suspended in TBME (300 mL) and stirred. Solvent was then removed by concentration in vacuo. The resulting solid was dried under vacuum overnight to afford the product (134 g, 76%). ¹H NMR (300 MHz, DMSO-d₆) δ 12.62 (s, 11H), 7.97 (s, 1H), 7.66-7.35 (m, 5H), 7.17 (s, 11H), 6.51 (s, 11H), 3.93-3.75 (m, 2H), 3.24 (td, J=11.3, 5.2 Hz, 2H), 2.82 (dt, J=10.4, 6.3 Hz, 1H), 1.70 (dt, J=10.1, 4.8 Hz, 4H). LCMS m/z 336.1 [M+H]⁺.

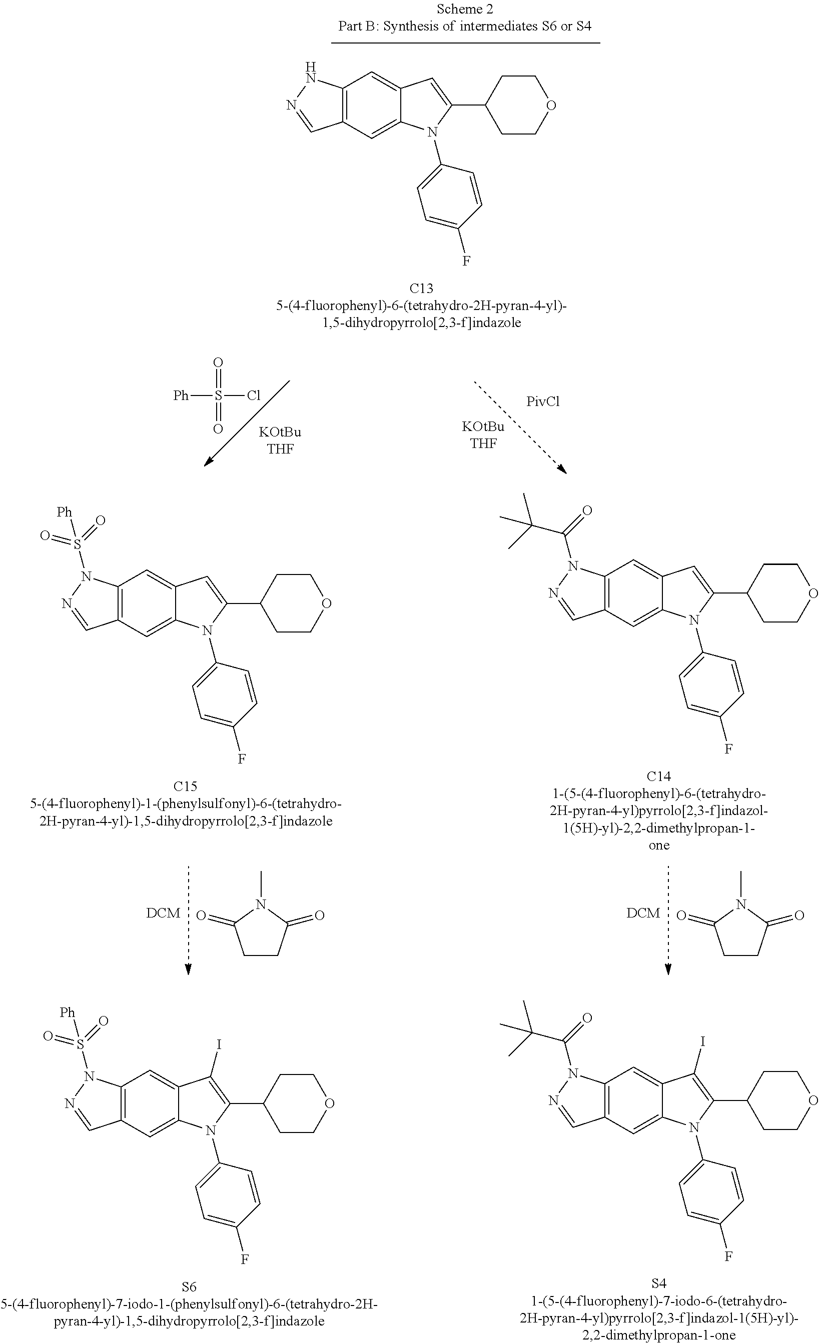

### Preparation of 1-[S-(4-fluorophenyl)-7-iodo-6-tetrahydropyran-4-yl-pyrrolo[2,3-f]indazol-1-yl]-2,2-dimethyl-propan-)-one (S4)

### Step 1. Synthesis of 1-[S-(4-fluorophenyl)-6-tetrahydropyran-4-yl-pyrrolo[2,3-f]indazol-1-yl]-2,2-dimethyl-propan-1-one (C14)

To a solution of 5-(4-fluorophenyl)-6-tetrahydropyran-4-yl-1H-pyrrolo[2,3-f]indazole C13 (10 g, 29.8 mmol) in THF (320 mL) at 0° C. was added KOtBu (7.4 g, 65.7 mmol) and the mixture allowed to stir for 5 min. 2,2-dimethylpropanoyl chloride (14.5 mL, 117.9 mmol) was added and the mixture allowed to stir for 1 h. Water (200 mL) and CH₂Cl₂ (250 mL) were added and the mixture extracted with additional dichloromethane (2×50 mL). The organic layer was dried over Na₂SO₄ and concentrated in vacuo. Purification by silica gel chromatography (Gradient: 0-5% EtOAc in Heptane) afforded the product as light yellow solid. 1-[5-(4-fluorophenyl)-6-tetrahydropyran-4-yl-pyrrolo[2,3-f]indazol-1-yl]-2,2-dimethyl-propan-1-one (10.7 g, 83%). ¹H NMR (400 MHz, Chloroform-d) δ 8.69 (s, 1H), 8.07 (s, 1H), 7.39 (dd, J=8.4, 4.9 Hz, 2H), 7.32 (d, J=8.3 Hz, 2H), 7.21 (s, 1H), 6.59 (s, 1H), 4.01 (dd, J=12.0, 4.1 Hz, 2H), 3.37 (t, J=11.7 Hz, 2H), 2.89-2.80 (m, 1H), 1.89 (qd, J=12.2, 4.1 Hz, 2H), 1.78 (d, J=13.0 Hz, 2H), 1.61 (d, J=1.3 Hz, 9H). LCMS m/z 420.3 [M+H]⁺.

### Step 2. Synthesis of 1-[5-(4-fluorophenyl)-7-iodo-6-tetrahydropyran-4-yl-pyrrolo[2,3-f]indazol-1-yl]-2,2-dimethyl-propan-1-one (S4)

1-iodopyrrolidine-2,5-dione (7.4 g, 31.2 mmol) was added portion-wise over 30 min to a solution of 1-[5-(4-fluorophenyl)-6-tetrahydropyran-4-yl-pyrrolo[2,3-f]indazol-1-yl]-2,2-dimethyl-propan-1-one C14 (10.7 g, 25.4 mmol) in CH₂Cl₂ (110 mL). The reaction was stirred at room temperature for 30 min. Purification by silica gel chromatography (Gradient: 0-5% EtOAc in Dichloromethane) resulted in an orange solid, which was triturated with heptane. Water (250 mL) was then added, and the mixture stirred vigorously for 30 min. The solid was filtered, washed with excess water, then dissolved in CH₂Cl₂ (250 mL). The solution was washed with water (250 mL) and the organic phase dried (phase separator) and concentrated in vacuo to afford the product as a light tan solid (11.7 g, 84%). ¹H NMR (400 MHz, Chloroform-d) δ 8.63 (s, 1H), 8.08 (s, 1H), 7.37-7.30 (m, 4H), 7.08 (s, 1H), 4.04 (dd, J=11.7, 4.2 Hz, 2H), 3.38 (t, J=11.8 Hz, 2H), 3.07 (t, J=12.6 Hz, 1H), 2.43 (qd, J=12.5, 4.3 Hz, 2H), 1.62 (s, 9H). LCMS m/z 546.33 [M+H]⁺.

### Preparation of 1-(benzenesulfonyl)-5-(4-fluorophenyl)-7-iodo-6-tetrahydropyran-4-yl-pyrrolo[2,3-f]indazole (S6)

### Step 1. Synthesis of 1-(benzenesulfonyl)-5-(4-fluorophenyl)-6-tetrahydropyran-4-yl-pyrrolo[2,3-f]indazole (C15)

To a solution of 5-(4-fluorophenyl)-6-tetrahydropyran-4-yl-1H-pyrrolo[2,3-f]indazole C13 (10 g, 29.8 mmol) in THF (120 mL) at 0° C. was added KOtBu (4.2 g, 37.3 mmol) and the mixture stirred for 10 min. Benzene sulfonyl chloride (4.4 mL, 34.5 mmol) was added, and the mixture stirred for 1 h at 0° C., then for an additional 1 h at room temperature. The mixture was concentrated in vacuo, and then saturated NH₄Cl and CH₂Cl₂ were added. The organic layer was separated, and dried. Purification by silica gel chromatography (Gradient: 0-60% CH₂Cl₂ in EtOAc) afforded the product as a white solid, containing around 5% of C13 (11.8 g, 83%). ¹H NMR (300 MHz, Chloroform-d) δ 8.38 (t, J=1.0 Hz, 1H), 8.14 (d, J=0.9 Hz, 1H), 8.04-7.93 (m, 2H), 7.57-7.47 (m, 1H), 7.46-7.38 (m, 2H), 7.38-7.30 (m, 3H), 7.15 (t, J=0.9 Hz, 1H), 6.62 (d, J=0.8 Hz, 1H), 4.08-3.94 (m, 2H), 3.37 (td, J=11.8, 2.3 Hz, 2H), 2.82 (ddt, J=11.5, 8.0, 3.9 Hz, 1H), 1.98-1.70 (m, 5H). LCMS m/z 476.2 [M+H]⁺.

### Step 2. Synthesis of 1-(benzenesulfonyl)-5-(4-fluorophenyl)-7-iodo-6-tetrahydropyran-4-yl-pyrrolo[2,3-f]indazole (S6)

To a solution of 1-(benzenesulfonyl)-5-(4-fluorophenyl)-6-tetrahydropyran-4-yl-pyrrolo[2,3-f]indazole C15 (151.8 g, 319.2 mmol) in CH₂Cl₂ (1.52 L) cooled to 0° C. was added 1-iodopyrrolidine-2,5-dione (74.5 g, 321.2 mmol), in 4 approximately equal portions over 45 min; additions were 15 min apart. After each addition a slight exotherm was observed, the internal temp. rose to −2° C. The reaction mixture was warmed to room temperature and stirred overnight. CH₂Cl₂ (500 mL) was added, and the reaction was stirred for 15 min. Water (1 L) was added, followed by 1 M aqueous sodium thiosulfate (200 mL). The mixture was stirred for 20 min, then the organic layer was separated, and the aqueous layer was extracted with CH₂Cl₂ (50 mL). Combined organic layers were washed successively with water, saturated aqueous sodium bicarbonate, and brine (1.5 L each). The organic layer was then dried (MgSO₄), filtered, and concentrated to afford a solid residue. The residue was treated with MTBE (500 mL), then stirred for 90 min. The resulting solid was isolated via filtration, washed with MTBE (2×200 mL), and dried under suction for 30 min. The solid was further dried under vacuum (2 mbar, 75° C.) for 30 min, to afford the product as pale, cream-colored crystals. 1-(benzenesulfonyl)-5-(4-fluorophenyl)-7-iodo-6-tetrahydropyran-4-yl-pyrrolo[2,3-f]indazole (181.4 g, 94%). ¹H NMR (400 MHz, DMSO-d₆) δ 8.51 (d, J=0.9 Hz, 1H), 8.06 (t, J=0.9 Hz, 1H), 7.87-7.80 (m, 2H), 7.71-7.63 (m, 1H), 7.62-7.45 (m, 6H), 7.25 (d, J=1.0 Hz, 1H), 3.96-3.85 (m, 2H), 3.22 (td, J=11.8, 1.9 Hz, 2H), 2.93 (tt, J=12.4, 3.6 Hz, 1H), 2.29 (qd, J=12.6, 4.4 Hz, 2H), 1.63 (dd, J=13.5, 3.5 Hz, 2H). 19F NMR (376 MHz, DMSO-d₆) δ −111.78. LCMS m/z 602.1 [M+H]⁺.

### Alternative Preparation of 1-[5-(4-fluorophenyl)-7-iodo-6-tetrahydropyran-4-yl-pyrrolo[2,3-f]indazol-1-yl]-2,2-dimethyl-propan-1-one (S4)

### Step 1. Synthesis of 5-bromo-6-(2-tetrahydropyran-4-ylethynyl)-1H-indazole (C2)

To reactor A under N2 was charged 5-bromo-6-(2-tetrahydropyran-4-ylethynyl)-1H-indazole C1 (12.0 kg), PdCl₂(PPh₃)₂, (0.26 kg), and CuI (0.35 kg). Reactor A was degassed (vacuum/nitrogen purges×2). To reactor B was charged EtOH (52.1 kg) (to aid in the transfer of trimethyl((tetrahydro-2H-pyran-4-yl)ethynyl)silane), and degassed with (vacuum/nitrogen purges×2). To reactor A was charged trimethyl((tetrahydro-2H-pyran-4-yl)ethynyl)silane (7.42 kg) and EtOH (4.7 kg). To reactor A was charged 45 wt % KOH (9.72 kg) and EtOH (4.6 kg) (to aid in the transfer of the 45 wt % KOH). The agitator was started in Reactor A, the vessel was then degassed (vacuum/nitrogen purges×4), and the contents of Reactor A were heated to 75±5° C. The reaction was held at 76.5 to 77.0° C. for 2 h, and then cooled to 40.1° C. over 20 min. The contents of reactor A were concentrated to a volume of 24 L by vacuum distilled with the maximum temperature of 35.1° C. The contents of reactor A were adjusted to 13.5° C. To a drum was added water (73.9 kg) and concentrated HCl (4.1 kg). The HCl transfer line was rinsed with water (4.7 kg) and charged to the drum. The contents of the drum were mixed (0.5 M HCl solution). The 0.5 M HCl solution (73.9 kg) was transferred to Reactor A over 21 min to cause precipitation of 5-bromo-6-(2-tetrahydropyran-4-ylethynyl)-1H-indazole C2 and a maximum temperature of 20.9° C. (spec. 20±5° C.) during the addition. An aliquot of the slurry was taken and the pH was measured to be 2.0 with a calibrated pH probe. KOH (45 wt %, 0.3 kg) was charged to Reactor A to give a reaction temperature of 15.4° C. An aliquot of the slurry was taken and the pH was measured to be 10.3 with a calibrated pH probe. HCl (0.5 M, 1.2 kg) was transferred over 2 min to reactor A with a maximum temperature of 13.8° C. An aliquot of the slurry was taken and the pH was measured to be 6.03 with a calibrated pH probe. The contents of reactor A were adjusted to 22.1° C. and held for 1 h at 22.1° C. The contents of reactor A were filtered (filtration time 27 min) and washed with water (2×36 kg). The solids were dried on the filter for 50 min, then dried on trays at 50-55° C. for 16 h to afford the product C2.

### Step 2. Synthesis of 5-(4-fluorophenyl)-6-tetrahydropyran-4-yl-1H-pyrrolo[2,3-f]indazole (C3)

NaOtBu, 97% (39.2 g, 407.4 mmol, 2.1 equiv.) was added to a reactor. Ethanol (355.2 mL, 6 vols) was added (note: exothermic reaction), and the mixture was purged with nitrogen. 5-bromo-6-[2-(oxan-4-yl)ethynyl]-1H-indazole C2 (59.2 g, 194 mmol, 1 equiv.) was added at 20° C. to the reactor. 4-fluoroaniline (23.71 g, 20.3 mL, 213.4 mmol, 1.1 equiv.) was then added and the mixture degassed (vacuum and nitrogen purge cycles×3). t-BuXPhos Pd G1 (4.0 g, 5.82 mmol, 0.03 equiv.) at 20° C. was added and the mixture degassed again (vacuum and nitrogen purge cycles×3). The reactor was heated to 65° C. internal temperature for 2 h, then cooled to 60° C. AcOH (55.3 g, 52.8 mL, 921.5 mmol, 4.75 equiv.) at 60° C. was added (note: exothermic reaction, solids precipitate during addition) and the reaction allowed to stir at 60-63° C. for 2 h. The mixture was then cooled to 25° C. Dichloromethane (8 vol) was added to the mixture. 0.5 M NaOH (5 vol) was added and the phases were stirred vigorously for 20 minutes. Additional 0.5 M NaOH was added to adjust the pH to pH 6-7. The phases were separated, and the aqueous phase was separated and extracted with dichloromethane (4 vol). The organic phases were combined, and distilled to ˜ 3 vol. Additional dichloromethane (6 vol) was added and the distillation to 3 vol. repeated. Addition of dichloromethane, then distillation was repeated until the residual EtOH was reduced to below 1% by NMR. The residual solution of 3 vol dichloromethane was heated to 38° C. Heptane (3 vol) was added and the mixture was stirred for 1 h, then cooled to 20° C. over 3 h. The resulting slurry was filtered and the filter cake washed with 1:1 v/v dichloromethane:heptane. The product was dried under vacuum at 45° C. to afford the product as a white solid (75% yield).

### Step 3. Synthesis of 1-[5-(4-fluorophenyl)-6-tetrahydropyran-4-yl-pyrrolo[2,3-f]indazol-1-yl]-2,2-dimethyl-propan-1-one (C14)

To reactor A under nitrogen was charged 5-(4-fluorophenyl)-6-tetrahydropyran-4-yl-1H-pyrrolo[2,3-f]indazole C13 (8.3 kg) and THF (99.4 kg). The agitator was started in Reactor A. Compound C13 dissolved and the solution was cooled to 1.7° C. KOtBu in THF (15.9 kg) was charged to reactor A over 9 min (temp. range during addition 0.2° C. to 1.6° C.). The transfer line was rinsed with THF (1.0 kg) and transferred to reactor A. The contents of reactor A were stirred for 10 min at 1.6° C. Pivaloyl chloride (3.3 kg) was charged over 32 min to reactor A with the maximum temperature reaching 2.3° C. The transfer line was rinsed with THF (0.5 kg) and transferred to reactor A. The contents of reactor A were held at 0.7° C. to 2.1° C. for 1 h. To a drum was charged NaHCO₃ (2.3 kg) and water (32.0 kg). The contents were briefly mixed to dissolve the NaHCO₃. The contents of reactor A were warmed to 19.0° C. over 2 h 10 min. The NaHCO₃ solution was charged to reactor A over 10 min (max. temp. during addition 19.4° C.). MTBE (29.3 kg) was charged to reactor A. The contents of reactor A were stirred at 25±5° C. for 15 min. The agitator was stopped and the phases separated for 33 min. The aqueous phase was removed. The agitator in reactor A was started. To a drum was added sodium chloride (6.2 kg) and water (26.1 kg). The drum was stirred to give a solution. The brine solution was transferred to reactor A. The contents were stirred for 19 min at 25±5° C. The agitator in reactor A was stopped and the phases settled for 20 min. The aqueous phase was removed. The agitator was started and the organic phase was concentrated by vacuum distillation to 30 L with the maximum distillation temperature of 26.2° C. To reactor A was charged n-heptane (21.9 kg). The contents of reactor A were concentrated to 30 L by vacuum distillation (maximum temperature 25.8° C.). To reactor A was charged n-heptane (21.8 kg) over 17 min. The contents of reactor A were concentrated to 30 L by vacuum distillation (maximum temperature 29.3° C.). To reactor A was charged n-heptane (23.0 kg) over 16 min. The contents of reactor A were stirred at 20±5° C. for 1 h. The slurry was filtered. To reactor A was charged n-heptane (11.2 kg) and transferred to the filter. This was repeated with another n-heptane (11.2 kg) rinse. The cake was dried under nitrogen pressure for 5 h and then loaded into trays and dried for 3 days to afford the product 1-[5-(4-fluorophenyl)-6-tetrahydropyran-4-yl-pyrrolo[2,3-f]indazol-1-yl]-2,2-dimethyl-propan-1-one (C14) as a solvate with THF (5 wt %) by ¹H NMR (6.9 kg, 68%, brown solid).

### Step 4. Synthesis of 1-[S-(4-fluorophenyl)-7-iodo-6-tetrahydropyran-4-yl-pyrrolo[2,3-f]indazol-1-yl]-2,2-dimethyl-propan-1-one (S4)

To reactor A under nitrogen was added 1-[5-(4-fluorophenyl)-6-tetrahydropyran-4-yl-pyrrolo[2,3-f]indazol-1-yl]-2,2-dimethyl-propan-1-one C14 (4.75 kg) and CH₂Cl₂ (29 L). The agitator was started and the jacket was set at −10° C. The solution was cooled to ≤5.0° C. and N-iodosuccinimide (2.73 kg) was added in three equal portions. At 3.0° C. the 1^{st} portion was added and gave an exotherm to 4.1° C. After 19 min the reaction temperature had cooled to 0.9° C. The 2^{nd} portion was added at 0.9° C. with an exotherm to 2.3° C. After 15 min, the reaction temperature had cooled to 1.4° C. The 3^{rd} portion was added at 1.4° C. with an exotherm to 2.1° C. CH₂Cl₂ (1 L) was charged to reactor A to rinse the N-iodosuccinimide. The jacket temperature was set at 0° C. and the reaction was stirred for 50 min with a final reaction temperature of 3.2° C. To a container was charged sodium thiosulfate pentahydrate (0.85 kg) and water (14.5 L). The contents were mixed to give a solution. The sodium thiosulfate solution (room temperature) was charged in portions to the reaction solution (3.4° C., jacket temperature 0° C.) over 8 min to give an exotherm to 11.6° C. The mixture was warmed to 20° C. stirred for 15 min. The agitator was stopped to let the phases separate for 35 min. The aqueous phase was removed and back extracted with CH₂Cl₂ (5 L). The mixture was stirred 10 min at 20° C. and the agitator was stopped. The phases settled for 10 min and the aqueous phase was removed. The organic phases were combined and charged back to reactor A. The agitator was started. To a container was charged KHCO₃ (0.90 kg) and water (14.1 L). The contents were mixed to give a solution. The KHCO₃ aq. solution was added to reactor A and stirred for 10 min at 20° C. The agitator was stopped and an emulsion had formed. The phases separated overnight and the aqueous phase was removed. The organic phase was charged back to the reactor and rinsed in with CH₂C2 (1 L). A container was charged NaCl (3.0 kg) and potable water (12.0 L). The contents were mixed to dissolve and the brine solution was transferred to reactor A. The contents of reactor A were mixed for 10 min at 20° C. The agitator was stopped and an emulsion had formed. After settling for 2 h the majority of the organic CH₂Cl₂ bottom phase was removed leaving behind about 18 L of emulsion. Water (7.5 L) was added to reactor A with slow stirring (50 rpm) this diluted the brine wash from 20 wt % to approximately 12 wt %. The phases separated in 20 min and the CH₂Cl₂ bottom layer was removed. The organic phase was split in half and concentrated in two flasks. Each flask was concentrated to 5 volumes. To each flask was charged MeOH (10 L) in portions and distilled to 4 volumes. To each flask was charged MeOH (4 L) and distilled to 2 volumes. The contents of each flask were cooled to 0-5° C. and stirred for 1.5 h. Contents of the two flasks were combined into one filter and filtered quickly. The filter cake was washed with 0-10° C. MeOH (2×5 L) and filtered fast. The cake was deliquored for 1 h under vacuum filtration and then loaded into drying trays. The solid was dried overnight at 45° C. in drying trays to afford S4 as a brown solid (5.75 kg, 8.98 wt % solvate).

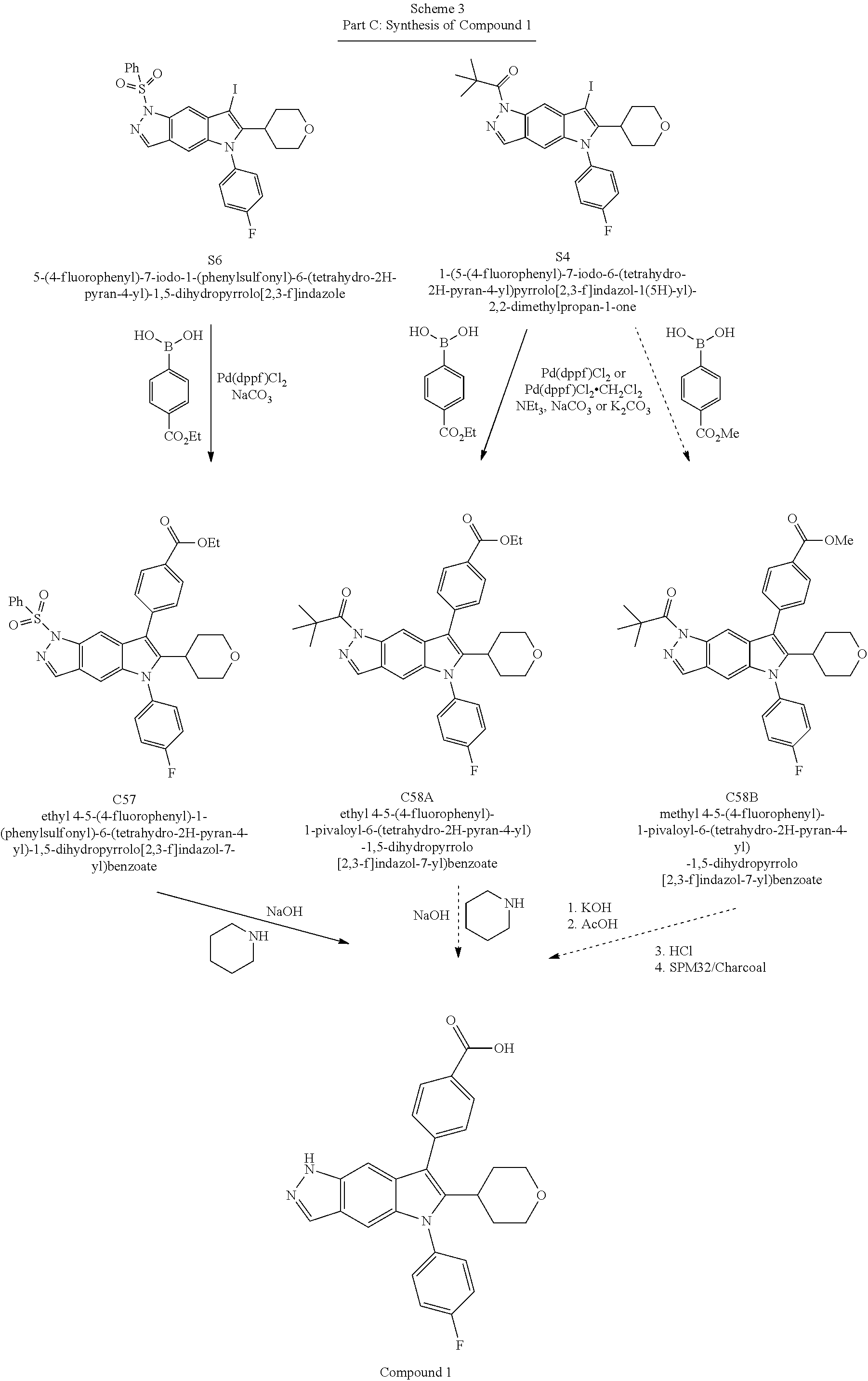

### Preparation of 4-[5-(4-fluorophenyl)-6-tetrahydropyran-4-yl-1H-pyrrolo[2,3-f]indazol-7-yl]benzoic acid (Compound 1) from S6

### Step 1. Synthesis of ethyl 4-[1-(benzenesulfonyl)-5-(4-fluorophenyl)-6-tetrahydropyran-4-yl-pyrrolo[2,3-f]indazol-7-yl]benzoate (C57)

A mixture of 1-(benzenesulfonyl)-5-(4-fluorophenyl)-7-iodo-6-tetrahydropyran-4-yl-pyrrolo[2,3-f]indazole S6 (103.8 g, 172.6 mmol), (4-ethoxycarbonylphenyl)boronic acid (67 g, 345.4 mmol), Pd(dppf)Cl₂ (6.4 g, 7.8 mmol) and Na₂CO₃ (270 mL of 2 M, 540 mmol) in 1,4-dioxane (1 L) was purged with nitrogen for 20 min, then heated at 90° C. for 1 h. The mixture was filtered through Celite®, washing with EtOAc (500 mL). The filtrate was concentrated to dryness in vacuo. EtOAc (1 L) and water (300 mL) were added. The organic layer was separated and filtered through Celite®. The organic layer was then washed with 1 M NaOH (300 mL×2), and brine. The organic layer was dried, and concentrated in vacuo. The residue was dissolved in CH₂Cl₂ (200 mL) and the solution was purified by silica gel chromatography. (Column: 3 kg Silica gel. Gradient: 0-100% EtOAc in heptane) to afford the product as a white, foamy solid (˜102 g). TBME (550 mL) was added, and the suspension was allowed to stir at room temperature for 1 h. The solid was filtered (washing with 200 mL MTBE). CH₂Cl₂ (300 mL) and EtOAc (400 mL) were added to afford a clear solution which was treated with MP-TMT Pd resin (45 g) and allowed to stir overnight. The suspension was filtered, and the filtrate concentrated in vacuo to afford the product as a white solid (96 g, 89%). ¹H NMR (300 MHz, Chloroform-d) δ 8.33-8.22 (m, 2H), 8.15 (d, J=0.8 Hz, 1H), 8.10 (t, J=0.9 Hz, 1H), 7.91 (dd, J=8.4, 1.3 Hz, 2H), 7.65-7.56 (m, 2H), 7.56-7.46 (m, 1H), 7.46-7.35 (m, 4H), 7.35-7.23 (m, 2H), 7.06 (d, J=1.0 Hz, 1H), 4.49 (q, J=7.1 Hz, 2H), 3.86 (dd, J=11.4, 3.5 Hz, 2H), 3.22 (t, J=11.0 Hz, 2H), 3.05 (ddd, J=12.2, 8.9, 3.3 Hz, 1H), 1.83 (qd, J=12.6, 4.3 Hz, 2H), 1.64 (s, 2H), 1.49 (t, J=7.1 Hz, 3H). LCMS m/z 624.3 [M+H]⁺.

### Step 2. 4-[5-(4-fluorophenyl)-6-tetrahydropyran-4-yl-1H-pyrrolo[2,3-f]indazol-7-yl]benzoic acid (Compound 1)

Piperidine (54 mL, 546.0 mmol) and NaOH (1350 mL of 1 M, 1.350 mol) were added to a solution of ethyl 4-[1-(benzenesulfonyl)-5-(4-fluorophenyl)-6-tetrahydropyran-4-yl-pyrrolo[2,3-f]indazol-7-yl]benzoate C57 (170 g, 272.6 mmol) in THF (1800 mL) and MeOH (1800 mL) and the mixture was heated to 50° C. for 3.5 h. Upon cooling, HCl (700 mL of 2 M, 1.40 mol) was added to adjust the mixture to pH=2. The solvent volume was reduced (by˜3 L) by concentration in vacuo. The light yellow precipitate was filtered off, washing the filter cake with water (×3), TBME (250 mL×2) and EtOAc (250 mL×2). The solid filter cake was dried under vacuum. The solid was then dissolved in EtOAc (1.2 L) and the solution heated to reflux for 10 min. ˜600 mL of solvent was removed by concentration under vacuum. An additional 600 mL of EtOAc was added and the process of refluxing for 10 min followed by removal of 1 L of solvent was repeated. Finally, EtOAc (1 L) was added and the mixture was heated at reflux for 2 h. Upon cooling overnight, the resulting solid was filtered off, washing with EtOAc (1×). This solid was then dried under vacuum at 60° C. for 4 h affording the product as a white solid (97.4 g, 78%). ¹H NMR (400 MHz, DMSO-d₆) δ 13.01 (s, 1H), 12.61 (s, 1H), 8.17-8.05 (m, 2H), 8.01 (d, J=1.0 Hz, 1H), 7.69-7.58 (m, 4H), 7.57-7.45 (m, 2H), 7.31-7.23 (m, 1H), 7.08 (d, J=1.1 Hz, 1H), 3.73 (dt, J=11.2, 3.1 Hz, 2H), 3.20-2.92 (m, 3H), 1.66 (h, J=4.2 Hz, 4H). LCMS m/z 456.0 [M+H]⁺.

### Preparation of 4-[5-(4-fluorophenyl)-6-tetrahydropyran-4-yl-1H-pyrrolo[2,3-f]indazol-7-yl]benzoic acid (Compound 1) from S4

### Step 1. Synthesis of ethyl 4-[1-(2,2-dimethylpropanoyl)-5-(4-fluorophenyl)-6-tetrahydropyran-4-yl-pyrrolo[2,3-f]indazol-7-yl]benzoate (C58)

A mixture of 1-[5-(4-fluorophenyl)-7-iodo-6-tetrahydropyran-4-yl-pyrrolo[2,3-f]indazol-1-yl]-2,2-dimethyl-propan-1-one S4 (1.0 g, 1.83 mmol), (4-ethoxycarbonylphenyl)boronic acid (556.9 mg, 2.87 mmol), and Pd(dppf)Cl₂ (76.3 mg, 0.09 mmol) was placed under a nitrogen atmosphere. 1,4-dioxane (8.8 mL) and sodium carbonate (3.2 mL of 2 M, 6.4 mmol) were added and the mixture was heated at 90° C. for 30 min. Purification by silica gel chromatography (0-5% EtOAc in CH₂Cl₂) gave a light tan solid. Minimal Et₂O and heptane were added to the solid, and the white solid precipitate was filtered off. The solid was dissolved in dichloromethane (ca. 25 mL). MP-TMT resin (1.1 g) was added and the mixture stirred for 1 h at room temperature. The resin was filtered off and the filtrate concentrated in vacuo to afford the product as a white solid (681.7 mg, 62%). ¹H NMR (400 MHz, Chloroform-d) δ 8.45 (s, 1H), 8.21 (d, J=7.8 Hz, 2H), 8.08 (s, 1H), 7.58 (d, J=8.0 Hz, 2H), 7.46 (dd, J=8.0, 4.9 Hz, 2H), 7.35 (t, J=8.2 Hz, 2H), 7.12 (s, 1H), 4.48 (q, J=6.9 Hz, 2H), 3.86 (dd, J=11.3, 4.2 Hz, 2H), 3.23 (t, J=11.7 Hz, 2H), 3.09-2.99 (m, 1H), 1.90-1.77 (m, 2H), 1.64 (d, J=13.2 Hz, 2H), 1.58 (s, 9H), 1.48 (t, J=7.1 Hz, 3H). LCMS m/z 568.5 [M+H]⁺.

### Step 2. Synthesis of 4-[S-(4-fluorophenyl)-6-tetrahydropyran-4-yl-1H-pyrrolo[2,3-f]indazol-7-yl]benzoic acid (Compound 1)

NaOH (6 mL of 1 M, 6.0 mmol) and piperidine (260 μL, 2.629 mmol) were added to a solution of ethyl 4-[1-(2,2-dimethylpropanoyl)-5-(4-fluorophenyl)-6-tetrahydropyran-4-yl-pyrrolo[2,3-f]indazol-7-yl]benzoate C58 (682 mg, 1.20 mmol) in THF (14 mL) and MeOH (7 mL). The mixture was heated at 50° C. for 1 h. The solvent was concentrated, and the residue re-dissolved in minimal water. HCl (6 mL of 1 M, 6.0 mmol) was added and a precipitate formed. The solid was filtered off and washed with excess water to afford the product as an off-white solid. (455.7 mg, 83%). ¹H NMR (400 MHz, DMSO-d₆) δ 13.02 (s, 1H), 12.60 (s, 1H), 8.11 (d, J=7.7 Hz, 2H), 8.00 (s, 1H), 7.63 (t, J=7.3 Hz, 4H), 7.51 (t, J=8.4 Hz, 2H), 7.26 (s, 1H), 7.07 (s, 1H), 3.73 (d, J=11.2 Hz, 2H), 3.15-3.07 (m, 2H), 3.05-2.96 (m, 1H), 1.72-1.61 (m, 4H). LCMS m/z 456.4 [M+H]⁺.

### Alternative Preparation of 4-[5-(4-fluorophenyl)-6-tetrahydropyran-4-yl-1H-pyrrolo[2,3-f]indazol-7-yl]benzoic acid (Compound 1) from S4

### Step 1. Synthesis of ethyl 4-[1-(2,2-dimethylpropanoyl)-5-(4-fluorophenyl)-6-tetrahydropyran-4-yl-pyrrolo[2,3-f]indazol-7-yl]benzoate (C58)

To reactor A under nitrogen was added S4 (5.42 kg), 4-methoxycarbonyl benzene boronic acid (1.786 kg), Na₂CO₃ (2.986 kg), 1,4-Dioxane (36 L), and potable water (12.5 L). The agitator was started and reactor A was degassed with one vacuum/nitrogen cycle. Nitrogen was bubbled via the bottom of the reaction mixture with stirring at room temperature while venting the nitrogen via the top of the reactor for 1 h. Pd(dppf)Cl₂—CH₂Cl₂ adduct (0.186 kg) was charged as a solid to reactor A. 1,4-Dioxane (1 L) was degassed (nitrogen bubbling for 5 min), and used to rinse the solids off the walls of reactor A. Reactor A was heated to 74° C.-78° C. for 3.5 h. The reaction was then held at 20° C. overnight, and then heated to 38.1° C. Potable water (24 L) was added to reactor A over 18 min, while maintaining the temperature at 36.0° C. to 38.1° C. The slurry was cooled to 20° C. over 2.5 h and filtered (filtration time 25 min). The cake was washed with potable water (2 L×2) and then was deliquored overnight. The wet filter cake solid and CH₂Cl₂ (25 L) was charged to reactor A. To a container was charged NaCl (1.1 kg) and potable water (9.9 kg). The contents were mixed to dissolve the NaCl. The brine solution was charged to reactor A. The agitator was started and the contents of reactor A were mixed at 22° C. for 15 min. The agitator was stopped and the layers separated for 22 min. The organic layer was removed (no emulsion). The aqueous layer was back extracted by charging CH₂Cl₂ (5 L) to reactor A. The agitator was started and mixed for 15 min. The agitator was stopped and the phases settled for 15 min. The CH₂Cl₂ layer was removed and combined with the 1^{st} CH₂Cl₂ layer. To reactor B was charged charcoal (1 kg) and the solution of product C58 in CH₂Cl₂. The agitator was started and stirred at room temperature for 23.5 h. A filter was set with Celite® plug and the contents of reactor B were filtered via the Celite® filter. The Celite® cake was washed with CH₂Cl₂ (6 L). The CH₂Cl₂ solution was concentrated to 2.5 volumes by vacuum distillation in two separate flasks. Heptanes (7 L) were charged to each flask while rotating, causing the formation of a thick slurry. Both flasks were held at room temperature overnight, and concentrated to 4 volumes. Each flask was cooled to 0-5° C., and rotated for 1 h. The contents of each flask were combined and filtered. The cake was washed with a CH₂Cl₂:heptanes (1:5) solution. The solids were loaded into trays and dried at 50° C. in a vacuum oven for 3 days, to afford the product C58 as a brown solid (5.3 kg, 88% yield, 8.0 wt % 1,4-dioxane solvate).

### Step 2. Synthesis of 4-[5-(4-fluorophenyl)-6-tetrahydropyran-4-yl-1H-pyrrolo[2,3-f]indazol-7-yl]benzoic acid (Compound 1)

### Part A. Hydrolysis

To reactor A under nitrogen was added ethyl 4-[1-(2,2-dimethylpropanoyl)-5-(4-fluorophenyl)-6-tetrahydropyran-4-yl-pyrrolo[2,3-f]indazol-7-yl]benzoate (C58) (5.2 kg), ethanol (26 L, 5 vol.), water (14.3 L, 2.7 equiv.), and 45% KOH (6.12 kg, 49.1 mol, 5.2 equiv.). The agitator was started and the reaction mixture was heated to 70-75° C. for 1 h. The reaction was cooled to room temperature and filtered via a plug of Celite®. Reactor A was rinsed with ethanol (5 L, 1 vol.) and used to rinse the Celite®. To reactor A was added acetic acid (2.968 kg, 49.5 mol, 5.2 equiv.) and water 17 L, 3.3 vol.). The acetic acid/water was heated to 46° C. and stirred at 200 rpm. The solution of C58 in ethanol was added over 22 min to the acetic acid/water to give a fine slurry. The temperature was 46.3° C. and the pH was 6.36. Acetic acid (1.176 kg, 19.7 mol, 2 equiv.) was added and the pH was 5.86 measured with a pH probe. The jacket was set with the following profile to hold at 50° C. for 9 h, cool to 20° C., and hold at 20° C. overnight. The slurry was stirred at 20° C. for 6 h before filtering. The slurry was filtered for 24 h. Water was charged to wash the cake (16 L, 3 vol.), which was filtered for an additional day to afford Compound 1 as a potassium salt (brown solid, approximately 80% yield).

### Part B. Free Acid Formation

To reactor A was added the wet 4-[5-(4-fluorophenyl)-6-tetrahydropyran-4-yl-1H-pyrrolo[2,3-f]indazol-7-yl]benzoic acid (Compound 1) potassium salt (3.4 kg). Potable water (44 L) was added to reactor A and the agitator was started. The mixture was stirred slowly at first and then at 133 rpm to give a nice slurry. 1M HCl (7.4 L) (0.1 equivalents excess based on an 80% isolated yield of the potassium salt of Compound 1) was charged to reactor A. Stirring was maintained for 3 h at 25° C., and then left overnight. The mixture was filtered on two filters by splitting the batch in half. After filtering for 8 h, the cake was washed with potable water (2 L) for each filter. The filtering continued overnight, and the cake was dried with vacuum filtration for 20 h. Compound 1 was dried under vacuum for 2 days at 50° C. and then for 2 days at 30° C. to afford the product (free acid) as a brown solid (3.4 kg, 80% yield).

### Part C. Palladium Scavenging

To reactor A under nitrogen was charged Compound 1 (3.4 kg, 7.47 mol), MeTHF (34 L), PhosphonicsS SPM32 (0.686 kg) (PhosphonicsS SPM32=3-Mercaptopropyl ethyl sulfide Silica, metal scavenging functionalized silica), and carbon (0.682 kg). The mixture was heated to 68° C. for 17 h with stirring. The mixture was cooled to 43° C. and filtered via a filter lined with a 2 inch silica gel pad. The silica was rinsed with MeTHF (6 L). A 2^{nd} treatment was carried out by charging SPM32 (0.68 kg), carbon (0.681 kg), and the filtrate of Compound 1 in MeTHF to a 100 L reactor under nitrogen. MeTHF (4 L) was used to aid in the transfer of the solution of Compound 1 in MeTHF back to the reactor. The stirring was initiated and the mixture was heated to 68° C. The mixture was stirred for 23 h, cooled to 50-60° C., and filtered as described above. This process was repeated two additional times. The filtrate was filtered via a 0.2 micron filter into a rotovap flask and concentrated to a wet solid. EtOH (8 L) was added and the vacuum distillation was continued to afford a solid. The solid was dried under vacuum at 50° C. overnight to afford Compound 1 (1.95 kg, 8% ethanol solvate).

### Part D. Drying Procedure

To a flask containing Compound 1 (1.95 kg, 8 wt % ethanol solvate) was added anhydrous CH₂Cl₂ (10 L). The mixture was distilled under vacuum to viscous slurry. CH₂Cl₂ (10 L) was added and the mixture was distilled under vacuum again, to give a wet solid. CH₂Cl₂ (10 L) was added to afford a slurry. The slurry was transferred to reactor A and additional CH₂Cl₂ (10 L) was used to transfer the residual contents of the flask to reactor A. The agitator was started, and the slurry was heated to 37° C., and held for 2 h at 35-37° C. The slurry was then cooled to 18° C. over 30 min, and held at 18° C. for 30 min. The slurry was filtered and washed with CH₂Cl₂ (2 L×2) at room temperature over 2 h. The filtered solid material was loaded into trays and dried in a vacuum oven at 70° C. overnight. The solids were broken apart into a fine powder, and dried for an additional 4 h to afford Compound 1 as a beige solid (1.36 kg, 72% yield, corrected for EtOH solvate, and 0.4% water).

### Alternative Preparation of 4-[5-(4-fluorophenyl)-6-tetrahydropyran-4-yl-1H-pyrrolo[2,3-f]indazol-7-yl]benzoic acid (Compound 1)

### Step 1. Synthesis of 5-bromo-6-((tetrahydro-2H-pyran-4-yl)ethynyl)-1H-indazole (C2)

Dispense 5-bromo-6-iodo-1H-indazole (C1) (45.0 g, 139.35 mmol, 1 equiv) in ethanol (270 mL, 6 vol). Charge trimethyl((tetrahydro-2H-pyran-4-yl)ethynyl)silane (27.95 g, 153.28 mmol, 1.1 equiv) and potassium hydroxide 40% w/v solution (41.05 mL, 292.63 mmol, 2.1 equiv).

Evacuate and sparge the reactor with nitrogen multiple times. Add palladium-bis(triphenylphosphine) dichloride (0.978 g, 1.39 mmol, 0.01 equiv) and copper iodide (1.34 g, 6.97 mmol, 0.05 equiv) to the reaction. Evacuate and sparge the reactor with nitrogen multiple times. Heat the reaction to 75° C. Upon reaction completion, cool the reaction and charge DCM (270 mL, 6 vol) followed by an aqueous ammonium chloride solution [9.2 wt %] (270 mL, 6 vol). Stop agitation and separate the layers. Wash the organic layer with an aqueous ammonium chloride [9.2 wt %] solution (270 mL, 6 vol). Charge hydrogen chloride [0.125M] (60 mL, 0.054 equiv) to reactor containing the organic layer to obtain a pH of 5-6 and stir for NLT 30 minutes. Stop agitation and separate layers. Wash the organic layer with an aqueous NaCl solution [8.7 wt %] (270 mL, 6 vol). Distill the organic layer, charge DCM (270 mL, 6 vol) and continue the distillation, repeat twice. Heat the resulting slurry to reflux and add cyclohexane [90 mL, 2 vol]. Cool the reaction to 20° C. over 5 hours. Filter the slurry and rinse the reactor with a 1:1 mixture of DCM/cyclohexane [1 vol]. Dry the wet cake in a vacuum oven at 45° C. with nitrogen bleed. The product, 5-bromo-6-((tetrahydro-2H-pyran-4-yl)ethynyl)-1H-indazole (C2) is isolated in 80% yield.

Examples of alternative reagents and solvents that can be used in Step 1 as described above are as follows:
Solvents: alcoholic solvents like 1-butanol, isopropyl alcohol (IPA), THF/alcohol mixtures, MeTHF/alcohols;
Base: NaOH, K₂CO₃, Na₂CO₃, Cs₂CO₃ NaOtBu, KOtBu;
Catalysts: Pd(PPh₃)₄;
Reaction without palladium using CuI or CuI/PPh₃ with KOH as base;
Reaction in DMF/with DBU as base with cat H₂O.

### Step 2. Synthesis of 5-(4-fluorophenyl)-6-(tetrahydro-2H-pyran-4-yl)-1,5-dihydropyrrolo[2,3-f]indazole (C13)

Add sodium tert-butoxide, 97% (99.2 g, 1032.2 mmol, 2.1 equiv) to reactor containing ethanol (900 mL, 6 vol). Degas and sparge solution with nitrogen multiple times. Add 5-bromo-6-((tetrahydro-2H-pyran-4-yl)ethynyl)-1H-indazole (C2) (150 g, 193.99 mmol, 1 equiv) and 4-fluoroaniline (60.08 g, 52.22 mL, 540.67 mmol, 1.1 equiv). Apply vacuum and nitrogen purge cycle 3 times

Add chloro(2-di-t-butylphosphino-2′,4′,6′-tri-i-propyl-1,1′-biphenyl)[2-(2-aminoethyl)phenyl] palladium(II) (11.796 g, 17.203 mmol, 0.035 equiv.) and degas and sparge with nitrogen NLT 3 times. Heat the reactor to 65° C. Upon reaction completion, add acetic acid (140.2 g, 133.65 mL, 2334.7 mmol, 4.75 equiv) at 60° C. and continue to stir for NLT 3 hours. Upon reaction completion, cool the reactor to 20° C. and add NaOH [0.5M] (900 mL, 6 vol) and DCM (600 mL, 4 vol) to reactor. Stop agitation and separate the layers. Back extract the aqueous layer with DCM. Combine the organic layers and distill the organic solution down to 3 volumes. Charge DCM (900 mL, 6 vol) to reactor and continue distillation; repeat the process two more times. Heat the reactor to 38° C. and add n-heptane (450 mL, 3 vol) over 2 hours. Cool the reactor down to 20° C. over 3 hours. Filter the slurry and rinse the wet cake a 1:1 ratio of DCM/n-heptane (1 volume). Dry the wet cake to vacuum oven set to 45° C. The product, 5-(4-fluorophenyl)-6-(tetrahydro-2H-pyran-4-yl)-1,5-dihydropyrrolo[2,3-f]indazole (C13), is isolated in 85% yield.

Examples of alternative reagents and solvents that can be used in Step 2 as described above are as follows:
Solvents: alcoholic solvents like 1-butanol, tert-butanol, isopropyl alcohol (IPA), tAmOH, THF, MeTHF, CPMe, Toluene, DMF, ACN, DMA, diglyme;
Base: NaOH, K₃PO₄, K₂CO₃, NaOtBu, KOtBu; NaOEt;
Catalysts in general all generations of catalysts should work: PdtBuXPhos G1-4 (tested); (PdOAc)₂Pd(cinnamyl)Cl₂ with ligands: BrettPhos, SPHos, XPhos, XantPhos, dppf, JosiPhos; cataCXium® A (Note: cyclization of N-(4-fluorophenyl)-6-((tetrahydro-2H-pyran-4-yl)ethynyl)-1H-indazol-5-amine to 5-(4-fluorophenyl)-6-(tetrahydro-2H-pyran-4-yl)-1,5-dihydropyrrolo[2,3-f]indazole;
Reagents: Acids, Lewis acids like copper salts and heat.

### Step 3. Synthesis of 1-(5-(4-fluorophenyl)-6-(tetrahydro-2H-pyran-4-yl)pyrrolo[2,3-f]indazol-1(5H)-yl)-2,2-dimethylpropan-1-one (C14)

Dissolve 5-(4-fluorophenyl)-6-(tetrahydro-2H-pyran-4-yl)-1,5-dihydropyrrolo[2,3-f]indazole (C13) (367.5 g, 1.09 mol, 1 equiv) in THF (5.15 L, 14 vol). Cool the reactor to −6° C. and add KOtBu [2M in THF] (0.71 L, 1.3 equiv). Stir the solution for at NLT 20 minutes. Add trimethylacetyl chloride (0.193 L, 1.43 equiv) to reactor at −6-0° C. and stir the content for 1 hour at 0° C. Upon reaction completion, heat the reactor to 18-20° C. over 1 hour. Add an aqueous solution of NaHCO₃ solution (101 g, 1.1 equiv 1.5 L, 4 vol. of water) and MtBE (1.5 L, 4 vol) to reactor. Stir the content for NLT 30 minutes at 20° C. Stop agitation and separate the layers. Prepare an aqueous NaCl solution by mixing NaCl (301 g, 4.7 equiv) in purified water (1.5 L, 4 vol). Add the aqueous NaCl solution to the organic layer and stir for NLT 30 minutes. Stop agitation and separate the layers. Add MP-TMT resin (73.5 g, 20 wt %) to reactor, heat the reactor to 50° C. and stir for NLT 12 hours. Filter the reactor content over a bed of celite and wash the celite with MtBE (0.7 L, 2 vol). Distill the organic filtrate down to 2-3 volumes. Add methanol (0.91 L, 2.5 vol) to reactor and heat the reactor to 60° C. Stir for 1 hour and add methanol (0.184 L, 0.5 vol) to the reactor. Cool the contents to 40° C. Stir the contents for 1 hour at 40° C. Add methanol (1.64 L, 4.5 vol) over 4 hours. Cool the contents to 10° C. over at least 4 hours and age the contents for at least 18 hours at 10° C. Filter the batch and rinse the wet cake with a mixture of methanol (1.38 L, 3.75 vol) and THF (0.46 L, 1.25 vol). Dry the wet cake at 45° C. under vacuum. The product, 1-(5-(4-fluorophenyl)-6-(tetrahydro-2H-pyran-4-yl)pyrrolo[2,3-f]indazol-1(5H)-yl)-2,2-dimethylpropan-1-one (C14), is isolated in 80% yield.

Examples of alternative reagents and solvents that can be used in Step 3 as described above are as follows:
Solvents: MeTHF, DCM;
Base: Li/Na/K OtBu, Na/K/LiOtAm.

### Step 4. Synthesis of 1-(5-(4-fluorophenyl)-6-(tetrahydro-2H-pyran-4-yl)pyrrolo[2,3-f]indazol-1(5H)-yl)-2,2-dimethylpropan-1-one (S4)

Dissolve 1-(5-(4-fluorophenyl)-6-(tetrahydro-2H-pyran-4-yl)pyrrolo[2,3-f]indazol-1(5H)-yl)-2,2-dimethylpropan-1-one (C14) (30.76 g, 73.3 mmol, 1 equiv, limiting reagent) in methylene chloride (307.6 mL, 10 vol). Cool the reactor down to −5° C. and add N-iodosuccinimide (18.23 g, 76.99 mmol, 1.05 equiv.) at −5.0-0° C. Stir reaction at −5° C. for NLT 30 minutes. Upon reaction completion, add an aqueous sodium thiosulfate solution (Na₂S₂O₃·5H₂O 9 g, 0.037 mmol, 0.5 equiv in purified water (0.1 L, 2.4 vol) to the reaction at 0° C. Stir the content for NLT 30 minutes at 0° C. followed by warm up to 20° C. Stop agitation and separate layers. Add an aqueous NaHCO₃ solution (NaHCO₃8.7 g, 0.1 mmol, 1.3 equiv dissolved in purified water (0.12 L, 3.7 vol) to the organic layer. Stir for NLT 30 minutes, stop agitation and separate layers. Add an aqueous NaCl solution (NaCl 20 g, 0.34 mmol, 4.7 equiv) in purified water (133 mL, 4.3 vol). Stir for NLT 30 minutes, stop agitation and separate layers. Distill the organic layer down to 2-3 volumes. Add THF (0.15 L, 5 vol) to reactor and distill down to 2-3 volumes, repeat 2-3 times. Add THF (up to 2 volumes) to the reactor to obtain a total of 4 volumes. Heat to slurry to internal temperature of 56-58° C. Add MeOH (0.061 L, 2 vol) at 56° C. over 1 hour to reactor. Cool the reactor content down to 52° C. and stir for NLT for 30 minutes. Add MeOH (0.25 L, 8 vol) over 3 hours at 52° C. to reactor. Cool the slurry down to 20° C. at a 5° C./h rate. Stir the reactor content at 20° C. for NLT 30 minutes. Filter the slurry and rinse the wet cake with MeOH (0.03 L, 1 vol) Dry the wet cake under vacuum at 60° C. The product, 1-(5-(4-fluorophenyl)-7-iodo-6-(tetrahydro-2H-pyran-4-yl)pyrrolo[2,3-f]indazol-1(5H)-yl)-2,2-dimethylpropan-1-one (S4), is isolated in 90% yield.

Examples of alternative solvents that can be used in Step 4 as described above are THF, MeTHF, CAN, EtOAc, DMF, dichloroethane (DCM).

### Step 5. Synthesis of methyl 4-(5-(4-fluorophenyl)-1-pivaloyl-6-(tetrahydro-2H-pyran-4-yl)-1,5-dihydropyrrolo[2,3-f]indazol-7-yl)benzoate (C58)

Add 1-(5-(4-fluorophenyl)-7-iodo-6-(tetrahydro-2H-pyran-4-yl)pyrrolo[2,3-f]indazol-1(5H)-yl)-2,2-dimethylpropan-1-one (S4) (10.0 g, 18.3 mmol, 1.0 eq.), 4-(methoxycarbonyl)-phenyl)boronic acid (3.80 g, 21.1 mmol, 1.15 eq.), and tetrahydrofuran (100 mL, 10 vol.) to a reactor and begin agitation. Prepare a solution of potassium carbonate in water by adding potassium carbonate (8.11 g, 58.7 mmol, 3.2 eq.) to water (70 mL, 7 vol.) at 25° C. in a separate vessel. Deoxygenate the mixture using three vacuum-nitrogen cycles. Add the aqueous potassium carbonate solution to the reactor. Deoxygenate the resulting biphasic mixture with three successive vacuum-nitrogen cycles. In a separate vessel, add triethylamine (74 mg, 0.73 mmol, 0.04 eq.) to a mixture of Pd(dppf)Cl₂ (0.30 g, 0.37 mmol, 0.020 eq.) and tetrahydrofuran (10 mL, 1 vol.). Deoxygenate using three vacuum-nitrogen cycles and the agitate the mixture for ˜1-2 h. Add the catalyst slurry to the reactor, rinsing forward with additional tetrahydrofuran (10 mL, 1 vol.) [Total tetrahydrofuran (120 mL, 12 vol) in reaction mixture], and perform an additional three vacuum-nitrogen cycles. Heat the reaction to 65° C. Upon reaction completion, cool the reactor contents to 55° C. and separate the layers. Add tetrahydrofuran (180 mL, 18 vol.) and Celite (100 wt %, 10.00 g) to the reactor and agitate at 55° C. for 1 hour. Filter the reaction mixture and rinse the cake with tetrahydrofuran (20 mL, 2 vol.). Charge SEM26 (2 g; 20 wt %) to the reactor and heat the mixture to 30-35° C. for NLT 18 hours. Filter the reaction mixture. Distill the filtrate down to 5 volumes. Add THF (150 mL, 15 vol.) and distill down to ˜7-8 volumes. Heat the reactor contents to 60-65° C. Cool reactor contents to 50° C. Add ethanol (140 mL, 14 vol.) over 2-3 hours at 50° C. and continue to stir for 30 min. Cool the mixture to 10° C. at a rate of 5° C./h. Stir the slurry at 10° C. for NLT 1 h and filter the mixture. Rinse the wet cake with ethanol (20 mL, 2×1 vol). Dry the solids under vacuum at 65° C. for NLT 12 h. The product, methyl 4-(5-(4-fluorophenyl)-1-pivaloyl-6-(tetrahydro-2H-pyran-4-yl)-1,5-dihydropyrrolo[2,3-f]indazol-7-yl)benzoate (C58), is isolated in 80% yield.

Examples of alternative reagents and solvents that can be used in Step 5 as described above are as follows:
Solvents: Dioxane, MeTHF, IPA, toluene, ACN, DMSO, EtOH;
Catalyst Monodentate ligands: PCy₃P(tBu)₃, DavePhos, SPhos Pd(PPh₃)₂Cl₂, Xphos, CataCXium; Pd(AmPhos)Cl₂, RuPhos;
Bidentate ligands: Pd(dippf)Cl₂, Pd(dtbpf)Cl₂, Pd(DPEPhos)Cl₂. Pd(dppf)Cl₂·CH₂Cl₂, Pd(Xantphos)Cl₂, Pd(dppb)Cl₂;
Base: K₂CO₃, Na₂CO₃. K₃PO₄.

### Step 6. Optional Recrystallization Procedure to Purge Residual Aryl Dimer

Charge the methyl 4-(5-(4-fluorophenyl)-1-pivaloyl-6-(tetrahydro-2H-pyran-4-yl)-1,5-dihydropyrrolo[2,3-f]indazol-7-yl)benzoate to a reactor. Add THF (9 vol.) and heat the reactor contents to 60° C. Cool reactor contents to 50° C. Add ethanol (18 vol.) over 2-3 hours. Stir the resulting thin slurry at 50° C. for 30 min. Cool the slurry to an internal temp. of 10° C. at a rate of 5° C./h. Stir the slurry at 10° C. for NLT 1 h Filter the mixture

Rinse the wet cake with ethanol (2×1-2 V) 1 (2×1-2 V) Dry the solids under vacuum at 65° C. for NLT 12 h. The product, methyl 4-(5-(4-fluorophenyl)-1-pivaloyl-6-(tetrahydro-2H-pyran-4-yl)-1,5-dihydropyrrolo[2,3-f]indazol-7-yl)benzoate, is isolated in 85% yield.

Add methyl 4-(5-(4-fluorophenyl)-1-pivaloyl-6-(tetrahydro-2H-pyran-4-yl)-1,5-dihydropyrrolo[2,3-f]indazol-7-yl)benzoate (C58) (25.1 g, 45.337 mmol, 1 equiv, limiting reagent) and THF (326.3 mL, 13 vol) to reactor. Add sodium hydroxide [2N] (5.44 g, 68.0 mL, 136.01 mmol, 3 equiv) to reactor and heat to 58° C. Upon reaction completion, cool reactor to 20° C. Add water (75.3 mL, 3 vol), acetic acid (10.89 g, 10.38 mL, 181.35 mmol, 4 equiv.) and 2-MeTHF (251 mL, 10 vols) to reactor and stir for NLT 30 minutes. Stop agitation and separate layers. Add water (75.3 mL, 3 vol) to organic layer and extract. Separate layers and add an aqueous 6.5 wt % sodium chloride solution (NaCl 8.2 g, 0.14 mmol, 3.1 equiv) in water (0.120 L, 4.7 vol) to the organic layer. Stir for NLT 30 minutes, then stop agitation and separate layers. Distill the organic layer down to 2-3 volumes. Add EtOH (0.176 mL, 7 vol) to reactor and continue distillation. Add EtOH (0.150 L, 6 vol) and water (25.1 mL, 1 vol) and distill the slurry down to 2-3 volumes. Add EtOH (0.150 L, 6 vol) and water (25.1 mL, 1 vol) to reactor and continue distillation down to 3 volumes. Add EtOH (0.150 L, 6 vol) and water (25.1 mL, 1 vol) to reactor and stir for NLT 30 minutes at 40° C. Cool the reactor down to 20-25° C. at a 5° C./h rate. Stir the reactor content for at least 30 minutes at 20° C. Filter the slurry and rinse the wet cake with a EtOH/H₂O 1:1 mixture (50 mL, 2 vol). Transfer the wet cake to vacuum oven set to 66° C. and dry the material for at NLT 12 hours. The product, 4-(5-(4-fluorophenyl)-6-(tetrahydro-2H-pyran-4-yl)-1,5-dihydropyrrolo[2,3-f]indazol-7-yl)benzoic acid (Compound 1), is isolated in 90% yield.

Examples of alternative reagents and solvents that can be used in Step 6 as described above are as follows:
Solvents: MeTHF, EtOH, MeOH, IPA;
Base: LiOH, NaOH, KOH
Work up: acetic acid, HCl.

In some embodiments, processes for preparing Compound 1 comprise reactions depicted in Schemes 4 and 5 below. Scheme 5 depicts a large-scale synthesis of Compound 1 that utilizes 1-(6-bromo-5-nitro-1H-indazol-1-yl)-2,2-dimethylpropan-1-one (A1) as the starting material. This process is expected to yield a solid form of Compound 1 or a pharmaceutically acceptable salt thereof at an amount of at least about 100 kg. Scheme 4 depicts the preparation of the starting material A1.

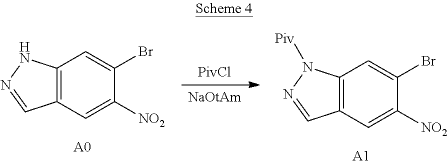

To A0, which is commercially available, (3.1 kg, 11.9 mol) in THF (35 L) at −26° C., sodium t-amylate (33.4 wt % in THF, 4.55 kg, 13.8 mol) was added over 15 minutes, and the mixture was rinsed with THF (300 mL). The mixture was re-cooled to −26° C. over 15 minutes and then pivaloyl chloride (Piv-Cl) (1.75 kg, 14.5 mol) was added over 4 minutes. The mixture was rinsed with THF (300 mL). The mixture was warmed to 15° C. over 55 minutes and held for 30 minutes. A solution of sodium bicarbonate (150 g) in water (2 L) was added, followed by additional water (9 L). The resulting biphasic slurry was concentrated under vacuum to ˜25-L volume then diluted with methanol (11.2 L). The slurry was heated to 40° C. for 30 minutes, diluted with water (11.3 L) over 30 minutes and then cooled to room temperature. A second run from 3.1 kg A0 was similarly performed. The two slurries were combined, filtered, and washed with 1:1 methanol:water (20 L). The solids were dried with heated nitrogen to afford A1 (7.69 kg, 23.6 mol, 99%) as a tan solid.

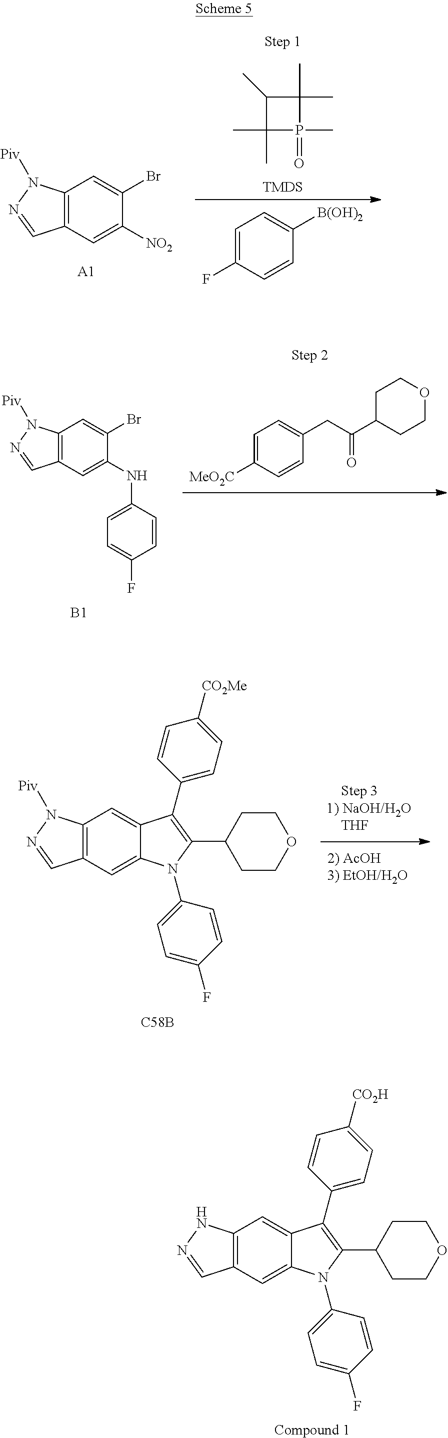

### Step 1: Synthesis of 1-(6-bromo-5-((4-fluorophenyl)amino)-1H-indazol-1-yl)-2,2-dimethylpropan-1-one (B1)

A1 (15.3 g, 46.911 mmol, 1 equiv.) is added to reactor. (4-fluorophenyl)boronic acid (8.533 g, 60.984 mmol, 1.3 equiv.) is added to reactor. 1,2,2,3,4,4-hexamethylphosphetane 1-oxide (1.127 g, 7.037 mmol, 0.15 equiv.) is added to reactor. Toluene (153 mL, 0.307 M, 10 volumes) is added to reactor. Dimethylsilyloxy(dimethyl)silane (TMDS) (18.904 g, 24.873 mL, 0.76 g/mL, 140.733 mmol, 3 equiv.) is added to reactor at 18.5° C. Heat reaction to 90° C. internal temperature. Once completion is obtained (around 7 hours, >97% conversion), set internal temperature to 20° C. Half saturated aqueous sodium bicarbonate (NaHCO₃) (76.5 mL, 0.613 M, 5 volumes) is added to reactor at 20-25° C. Tetrahydrofuran (THF) is added (2 volumes, 30 mL) and stirred for 15 minutes. Stirring is stopped and the phases are allowed to separate. The organic layer is washed with 5 volumes half saturated brine. The organic layer is then distilled down to 2 volumes. THF is added and distilled down to 1-2 volumes. This is repeated 3 times. THF is added to a total of 3 volumes. Methanol (MeOH) (45.9 mL, 1.022 M, 3 volumes) is added to reactor. The resulting slurry is heated to 55-60° C. internal temperature and then cooled down to 45-50° C. to obtain a seed bed. MeOH (92 mL, 6 volumes) is added over 180 minutes. The reactor is cooled down over 4 hours to 20-25° C. The slurry is filtered and the reactor is rinsed with MeOH. The rinse is dropped onto the wet cake. The wet cake is then transferred to a vacuum oven and dried at 50° C. to yield 1-(6-bromo-5-((4-fluorophenyl)amino)-1H-indazol-1-yl)-2,2-dimethylpropan-1-one (B1) as beige solid, expected yield is 70%. ¹H NMR (400 MHz, Chloroform-d) δ 8.76 (d, J=0.9 Hz, 1H), 7.87 (d, J=0.9 Hz, 1H), 7.24 (d, J=5.9 Hz, 2H), 7.18-6.97 (m, 4H), 5.97 (s, 1H), 1.54 (s, 9H), 1.43 (d, J=0.8 Hz, 1H).

### Step 2: Synthesis of methyl 4-(5-(4-fluorophenyl)-1-pivaloyl-6-(tetrahydro-2H-pyran-4-yl)-1,5-dihydropyrrolo[2,3-f]indazol-7-yl)benzoate (C58B)

B1 (0.50 g, 1.28 mmol, 1 equiv.) and methyl 4-(2-oxo-2-(tetrahydro-2H-pyran-4-yl)ethyl)benzoate (0.51 g, 1.95 mmol, 1.5 equiv.) are added to reactor. Potassium carbonate, 325 mesh (0.44 g, 2.5 equiv.) and 2-methyltetrahydrofuran (2-Me-THF) (5 mL, 10 volumes) are added to reactor. The reaction is degassed with nitrogen using 3 vacuum/purge cycles. Bis(tri-t-butylphosphine) Pd (0.033 g, 0.05 equiv.) is added and the reaction is degassed with nitrogen using 3 vacuum/purge cycles. The reaction is heated to 75° C. internal temperature. Once complete conversion is obtained, the internal temperature is set to 20° C. Water (2.5 mL, 5 volumes) is added to reactor at 20-25° C. and stirred for 15 minutes. Stirring is stopped and the phases are allowed to separate. 0.1 N HCl (2.5 mL, 5 volumes) is added to reactor at 20-25° C. and stirred for 15 minutes. Stirring is stopped and the phases are allowed to separate. The organic layer is distilled down to 2 volumes. THF (7 volumes) is added and the resulting solution is distilled down to 1-2 volumes, which is repeated 3 times. THE is added to a total of 15 volumes. Celite (100 wt %, 0.50 g) is added to the reactor and agitated at 55° C. for 1 hour. The THE rinse solution (2 mL, 4 volumes) is heated to 45-50° C. (in a separate reactor if desired). The reaction mixture is filtered, and rinsed forward with hot THF a couple of times (Each rinse equals 1 mL, 2 volumes). The filtrate is charged with the rinses back to the reactor. The mixture is heated to 30-35° C. 2-Mercaptoethyl ethyl sulfide silica (SEM26) (0.1 g; 20 wt %) is charged to the reactor. The mixture is heated to an internal temperature of 30-35° C. for no longer than 18 hours. The reaction mixture is filtered and rinsed forward with tetrahydrofuran a couple of times (Each rinse equals 1 mL, 2 volumes). The filtrate is charged with the rinses back to the reactor. The filtrate is concentrated down to a minimum volume (˜5 volumes). THF (7.5 mL, 15 vol.) is added and the volatiles are removed again to ˜7-8 volumes. The reactor contents are heated to 60-65° C. internal temperature. Reactor contents are cooled to 50° C. Ethanol (7 mL, 14 vol.) is added over 2-3 hours. The resulting thin slurry is stirred at 50° C. for 30 min. The slurry is cooled to an internal temperature of 10° C. at a rate of 5° C./h. The slurry is stirred at 10° C. for no longer than 1 h. The mixture is filtered. The reactor contents are rinsed twice with ethanol (2×1-2 volumes) and the rinse is dropped onto the wet cake. The wet cake was dried by pulling air through the filter for no longer than 30 minutes. The wet cake solids are transferred to a drying dish. The solids are dried under vacuum (nitrogen sweep, 20 mmHg) at 65° C. for 16 h to provide methyl 4-(5-(4-fluorophenyl)-1-pivaloyl-6-(tetrahydro-2H-pyran-4-yl)-1,5-dihydropyrrolo[2,3-f]indazol-7-yl)benzoate (C58B) with a 70% yield.

### Preparation of methyl 4-(2-oxo-2-(tetrahydro-2H-pyran-4-yl)ethyl)benzoate

The reactor was charged with methyl 4-(2-methoxy-2-oxoethyl)benzoate (500 mg, 2.401 mmol, 1 equiv.) and tetrahydrofuran (4.0 mL, 8 vol), followed by potassium tert-butoxide (2.8 mL, 1.0 M, 1.2 equiv.) at ambient temperature. The resulting slurry was transferred to a solution of oxane-4-carbonyl chloride (0.59 mL, 2 equiv.) and tetrahydrofuran (1.0 mL, 1 vol). The reaction was quenched with saturated aqueous ammonium chloride (5.0 mL, 10 Vols) and extract three times with ethyl acetate (5.0 mL, 10 vol). The combined organics was washed with 50% sat. aqueous sodium chloride (10.0 mL, 20 vols), then dried with sodium sulfate, filtered, and concentrated in vacuo to afford methyl 4-(1-methoxy-1,3-dioxo-3-(tetrahydro-2H-pyran-4-yl)propan-2-yl)benzoate. ¹H NMR (400 MHz, CDCl3) δ 8.03 (d, J=8.4 Hz, 2H), 7.42 (d, J=8.4 Hz, 2H), 4.95 (s, 1H), 3.92 (s, 3H), 3.75 (s, 3H), 3.40-3.27 (m, 2H), 3.14 (td, J=12.1, 2.0 Hz, 2H), 2.69 (tt, J=11.1, 4.1 Hz, 1H), 2.02-1.90 (m, 2H), 1.48-1.39 (m, 2H).

The reactor was charged with methyl 4-(1-methoxy-1,3-dioxo-3-(tetrahydro-2H-pyran-4-yl)propan-2-yl)benzoate (489 mg, 1.528 mmol, 1 equiv.), dimethyl sulfoxide (4.9 mL, 10 vol), and aqueous sodium chloride (0.68 mL, 4.5 M, 2.0 equiv.). The reaction mixture was heated to 150° C. for 3 hours, then cooled to room temperature. The reaction mixture was diluted with H₂O (4.9 mL, 10 vol) and extracted three times with ethyl acetate (4.9 mL, 10 vol). The combined organics was dried with sodium sulfate, filtered, and concentrated in vacuo to afford methyl 4-(2-oxo-2-(tetrahydro-2H-pyran-4-yl)ethyl)benzoate. ¹H NMR (400 MHz, CDCl3) δ 8.00 (d, J=8.3 Hz, 2H), 7.26 (d, J=8.4 Hz, 2H), 3.99 (dt, J=11.5, 3.5 Hz, 2H), 3.91 (s, 3H), 3.81 (s, 2H), 3.45-3.35 (m, 2H), 2.73-2.61 (m, 1H), 1.79-1.68 (m, 4H).

### Step 3: Synthesis of Compound 1

Methyl 4-(5-(4-fluorophenyl)-1-pivaloyl-6-(tetrahydro-2H-pyran-4-yl)-1,5-dihydropyrrolo[2,3-f]indazol-7-yl)benzoate (C58B) (25.1 g, 45.337 mmol, 1 equiv., limiting reagent) and THF (326.3 mL, 13 volumes) was added to reactor. Sodium hydroxide [2N] (5.44 g, 68.0 mL, 136.01 mmol, 3 equiv.) was added to reactor and heated to 58° C. Upon reaction completion, reactor was cooled to 20° C. Water (75.3 mL, 3 volumes), acetic acid (10.89 g, 10.38 mL, 181.35 mmol, 4 equiv.) and 2-MeTHF (251 mL, 10 volumes) were added to reactor and stirred for no longer than 30 minutes. Agitation was stopped and the layers allowed to separate. Water (75.3 mL, 3 vol) was added to the organic layer and extracted. Layers were separated and an aqueous 6.5 wt % sodium chloride solution (NaCl 8.2 g, 0.14 mmol, 3.1 equiv.) in water (0.120 L, 4.7 vol) was added to the organic layer. The reaction was stirred for no longer than 30 minutes, then agitation was stopped and the layers were separated. The organic layer was distilled down to 2-3 volumes. EtOH (0.176 mL, 7 volumes) was added to the reactor and distillation continued. EtOH (0.150 L, 6 volumes) and water (25.1 mL, 1 volume) were added, and the slurry was distilled down to 2-3 volumes. EtOH (0.150 L, 6 volumes) and water (25.1 mL, 1 volume) were added to reactor and distillation continued down to 3 volumes. EtOH (0.150 L, 6 volumes) and water (25.1 mL, 1 volume) were added to reactor and stirred for no longer than 30 minutes at 40° C. The reactor was cooled down to 20-25° C. at a 5° C./h rate. The reactor content was stirred for at least 30 minutes at 20° C. The slurry was filtered and the wet cake rinsed with a EtOH/H₂O 1:1 mixture (50 mL, 2 volumes). The wet cake was transferred to a vacuum oven set to 66° C. and the material dried for no longer than 12 hours. The product, 4-(5-(4-fluorophenyl)-6-(tetrahydro-2H-pyran-4-yl)-1,5-dihydropyrrolo[2,3-f]indazol-7-yl)benzoic acid (Compound 1), was isolated in 90% yield.

Examples of alternative reagents and solvents that can be used in the conversion of C58B to Compound 1 described above are as follows:
Solvents: MeTHF, EtOH, MeOH, IPA;
Base: LiOH, NaOH, KOH
Work up: acetic acid, HCl.

### Alternative Preparations of Compound 1 and Intermediate C13

The intermediate 1-(6-bromo-5-((4-fluorophenyl)amino)-1H-indazol-1-yl)-2,2-dimethylpropan-1-one (B1) described in Example 1 may be used as a starting material to prepare C13. As depicted in Schemes 1B-1C, C13 is a key intermediate in the synthesis of Compound 1. Accordingly, the present disclosure provides alternative preparations of Compound 1 and C13, where B1 is used as the starting material, as depicted in Scheme 6 below and as described as follows:

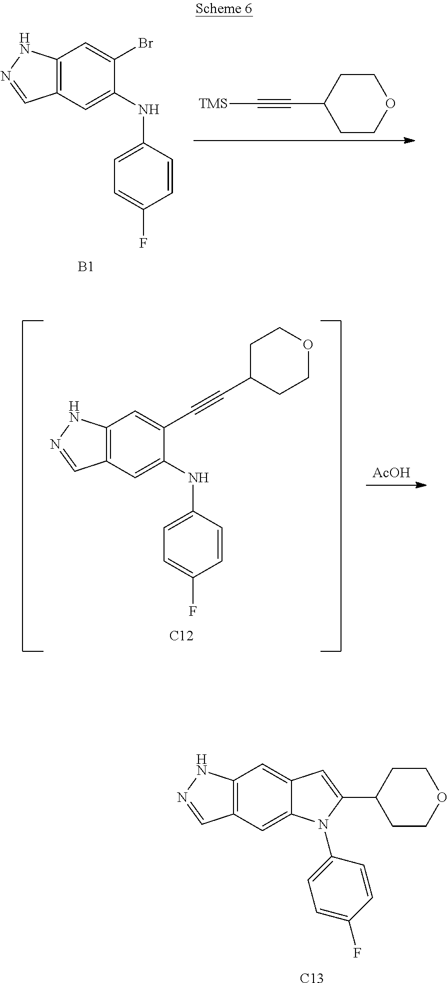

The reactor was charged with 6-bromo-N-(4-fluorophenyl)-1H-indazol-5-amine (6.3 g, 20.579 mmol, 1 equiv.), copper iodide 99.9% (0.274 g, 1.441 mmol, 0.07 equiv.) and bis(triphenylphosphine)palladium(II) dichloride (0.144 g, 0.206 mmol, 0.01 equiv.). The reaction mixture was charged with 2-propanol (50.4 mL, 0.408 M, 8 vol) and stirring was initiated. The system was evacuated and purged with nitrogen three times. Potassium hydroxide (2.887 g, 7.216 mL, 40 w/v %, 51.448 mmol, 2.5 equiv.) was added, followed by trimethyl((tetrahydro-2H-pyran-4-yl)ethynyl)silane (4.878 g, 26.753 mmol, 1.3 equiv.). The system was evacuated and purged with nitrogen three times. The reaction was heated to 75-80° C. Upon reaction completion, the mixture was charged with acetic acid (5.87 g, 5.596 mL, 1.049 g/mL, 97.752 mmol, 4.75 equiv.) and stirring was continued at 75-80° C. Upon reaction completion, the mixture was cooled down to 50° C. and water (50.4 mL, 0.408 M, 8 Vols) was slowly added. The reaction was cooled to 23° C. The solids were collected by filtration and the wet cake was washed with water. The material was dried under vacuum at 55° C. 5-(4-fluorophenyl)-6-(tetrahydro-2H-pyran-4-yl)-1,5-dihydropyrrolo[2,3-f]indazole was isolated in 94% yield.

Examples of alternative reagents and solvents that can be used in the conversion of B1 to C13 are:
Solvents: Other alcohol solvents such as 1-butanol, ethanol
Base: NaOH

Subsequent reaction steps for preparing Compound 1 are depicted in Schemes 1B and 1C and are also described in International Patent Application No. PCT/US2020/032832.

### Preparation of Compound 1 Form A

Methyl 4-(5-(4-fluorophenyl)-1-pivaloyl-6-(tetrahydro-2H-pyran-4-yl)-1,5-dihydropyrrolo[2,3-f]indazol-7-yl)benzoate (25.1 g, 45.337 mmol) was dissolved in THF (326.3 mL, 13 vol). Sodium hydroxide [2N] (5.44 g, 68.0 mL, 136.01 mmol, 3 equiv) was added and the mixture was heated to 55-60° C. Upon reaction completion, the reaction mixture was cooled to 20° C. and water (75.3 mL, 3 vol) and acetic acid (10.89 g, 10.38 mL, 181.35 mmol, 4 equiv.) was added thereto. 2-MeTHF (251 mL, 10 vols) was added and aqueous work up was performed. The organic layer was washed with water (75.3 mL, 3 vol) followed by a 6.5 wt % sodium chloride solution by dissolving NaCl (8.2 g, 0.14 mmol, 3.1 equiv) in water (0.120 L, 4.7 vol). The organic layer and solvent swap were distilled into ethanol. A mixture of EtOH (0.150 L, 6 vol) and water (25.1 mL, 1 vol) was added and distillation was continued; this step was repeated once. EtOH (0.150 L, 6 vol) and water (25.1 mL, 1 vol) were added to the reactor and the mixture was stirred at 40° C. The mixture was cooled to 20-25° C., and the product was isolated by filtration. Compound 1 was dried under vacuum at 66° C. with nitrogen bleed. Compound 1 was isolated in 90% yield with >99.8% area.

### Example 2. Solid Forms of Compound 1

X-Ray Powder Diffraction (XRPD): XRPD was performed with a Panalytical X'Pert³ Powder XRPD on a Si zero-background holder. The 2θ position was calibrated against a Panalytical Si reference standard disc.

TABLE 1
Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range | 3-40 |
| (° 2TH) | |
| Scan step time [s] | 18.87 |
| Step size | 0.0131 |
| (° 2TH) | |
| Test Time | 4 min 15 s |

Solid State NMR (ssNMR): Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS Tt saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz. All carbon, fluorine, and sodium spectra were referenced indirectly (through gyromagnetic ratios) to the upfield carbon peak of adamantane at 29.5 ppm.

Additional instrument information is provided if the alternative instrument was used for the form analysis.

1. Compound 1 Neat Form C

Synthetic Procedure: −10 mg DMSO solvate Form A was heated from RT to 300° C. at the heating rate of 10° C./min, followed by nitrogen purge cooling to the ambient temperature.

X-Ray Powder Diffraction (XRPD): FIG. 1A depicts an XRPD diffractogram of Compound 1 neat Form C. Table 2 provides the XRPD peaks, angles, and intensity % for Compound 1 neat Form C.

TABLE 2
XRPD peaks, angles and intensity % of Compound 1 neat Form C

| | Pos. | |
|---|---|---|
| Peak No. | [±0.2 °2θ] | Rel. Int. [%] |
| 1 | 19.0 | 100.0 |
| 2 | 9.4 | 41.0 |
| 3 | 15.4 | 18.7 |
| 4 | 21.1 | 16.0 |
| 5 | 18.2 | 14.8 |
| 6 | 19.6 | 12.3 |
| 7 | 20.1 | 10.7 |

Solid State NMR (ssNMR): FIG. 1B depicts a solid state ¹⁹F NMR spectrum for Compound 1 neat Form C. Table 3 recites ¹⁹F ssNMR chemical shift data for Compound 1 neat Form C.

TABLE 3
¹⁹F ssNMR chemical shift data of Compound 1 neat Form C

| | Chem Shift | |
|---|---|---|
| Peak No. | [ppm] | Intensity [rel] |
| 1 | −107.5 | 12.5 |

Thermogravimetric Analysis (TGA): TGA of Compound 1 neat Form C was measured using TA Discovery 550 TGA from TA Instrument. A sample with weight of approximately 1-5 mg was scanned from 25° C. to 290° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 1C showed minimum weight loss from ambient temperature up to 250° C.

Differential Scanning Calorimetry Analysis (DSC): DSC of Compound 1 neat Form C was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 346° C. When the run was completed, the data were analyzed by Trios and/or Universal Analysis software (TA Instruments, New Castle, DE). The thermogram of FIG. 1D showed two endothermic peaks at about 327 and 342° C.

2. Compound 1 Na Salt Form A

Synthetic Procedure: Compound 1 Na salt Form A was prepared by reacting 20 mg Compound 1 neat Form A with 1.76 mg NaOH in 3 mL acetone at RT stirring for 2-4 days (i.e., molar ratio of free form/counter ion at 1:1). The solids were filtered and air dried before the form analysis.

X-Ray Powder Diffraction (XRPD): FIG. 2A depicts an XRPD diffractogram of Compound 1 Na salt Form A. Table 4 provides the XRPD peaks, angles, and intensity % for Compound 1 Na salt Form A.

TABLE 4
XRPD peaks, angles and intensity
% of Compound 1 Na salt Form A

| | Pos. | |
|---|---|---|
| Peak No. | [±0.2 °2θ] | Rel. Int. [%] |
| 1 | 11.6 | 100.0 |
| 2 | 17.8 | 33.2 |
| 3 | 7.3 | 30.8 |
| 4 | 20.6 | 29.0 |
| 5 | 18.7 | 20.4 |
| 6 | 21.9 | 19.0 |
| 7 | 16.4 | 17.6 |
| 8 | 23.2 | 15.3 |
| 9 | 21.4 | 14.8 |

Thermogravimetric Analysis (TGA): TGA of Compound 1 Na salt Form A was measured using TA Discovery 550 TGA from TA Instrument. A sample with weight of approximately 1-5 mg was scanned from 25° C. to 370° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 2B showed 23.5% weight loss from ambient temperature up to 250° C.

Differential Scanning Calorimetry Analysis (DSC): DSC of Compound 1 Na salt Form A was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 375° C. When the run was completed, the data were analyzed by Trios and/or Universal Analysis software (TA Instruments, New Castle, DE). The thermogram of FIG. 2C showed an endothermic peak at about 132° C.

3. Compound 1 Na Salt Form B

Synthetic Procedure: Compound 1 Na salt Form B was prepared via reacting 20 mg Compound 1 neat Form A with 3.52 mg NaOH in 6 mL ethyl acetate at RT stirring for 2-4 days (i.e., molar ratio of free form/counter ion at 1:2). The solids were filtered and air dried before the further analysis.

X-Ray Powder Diffraction (XRPD): FIG. 3A depicts an XRPD diffractogram of Compound 1 Na salt Form B. Table 5 provides the XRPD peaks, angles, and intensity % for Compound 1 Na salt Form B.

TABLE 5
XRPD peaks, angles and intensity
% of Compound 1 Na salt Form B

| | Pos. | |
|---|---|---|
| Peak No. | [±0.2 °2θ] | Rel. Int. [%] |
| 1 | 8.9 | 100 |
| 2 | 17.8 | 3.6 |
| 3 | 26.9 | 2.6 |
| 4 | 3.1 | 2.4 |

Thermogravimetric Analysis (TGA): TGA of Compound 1 Na salt Form B was measured using TA Discovery 550 TGA from TA Instrument. A sample with weight of approximately 1-5 mg was scanned from 25° C. to 375° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 3B showed 9.0% weight loss from ambient temperature up to 300° C.

Differential Scanning Calorimetry Analysis (DSC): DSC of Compound 1 Na salt Form B was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 375° C. When the run was completed, the data were analyzed by Trios and/or Universal Analysis software (TA Instruments, New Castle, DE). The thermogram of FIG. 3C showed three endothermic peaks at about 85, 100, and 252° C.

4. Compound 1 Na Salt Form C

Synthetic Procedure: 1 g of Compound 1 neat Form A was added with 13.413 g of poly(ethylene glycol) 400 (PEG400) aqueous solution (PEG400 and water at 35:65 w/w). 2.238 g of 1 eq. NaOH was added dropwise to the solution while stirring. The solution was stirred at 400 rpm at RT or 4° C. covered with aluminum foil paper for 5 days. The precipitate was collected for form analysis.

˜40 mg Compound 1 neat Form A was weighed into a 4 mL vial, followed by adding 870 mg 5 wt % TPGS aqueous solution and 88 μl NaOH 1 N solution. The sample was left to stir in the cold room at 5° C. for 2 days. Then the solid was collected via centrifugal filtering for further analysis.

X-Ray Powder Diffraction (XRPD): XRPD spectra were recorded at room temperature in transmission mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 2θ with a step size of 0.0131303° and 49 s per step. FIG. 4A depicts an XRPD diffractogram of Compound 1 Na salt Form C. Table 6 provides the XRPD peaks, angles, and intensity % for Compound 1 Na salt Form C.

TABLE 6
XRPD peaks, angles and intensity
% of Compound 1 Na salt Form C

| | Pos. | |
|---|---|---|
| Peak No. | [+0.2 °2θ] | Rel. Int. [%] |
| 1 | 19.2 | 100.0 |
| 2 | 19.7 | 32.0 |
| 3 | 11.9 | 26.1 |
| 4 | 17.1 | 18.2 |
| 5 | 20.7 | 16.3 |
| 6 | 23.9 | 15.9 |
| 7 | 20.8 | 15.5 |
| 8 | 26.6 | 15.1 |
| 9 | 9.2 | 14.8 |
| 10 | 27.2 | 13.0 |
| 11 | 26.7 | 12.8 |
| 12 | 10.4 | 12.6 |
| 13 | 17.7 | 12.6 |
| 14 | 13.3 | 10.2 |

Solid State NMR (ssNMR): FIG. 4B depicts a solid state ¹³C NMR spectrum for Compound 1 Na salt Form C. Table 7 recites ¹³C ssNMR chemical shift data for Compound 1 Na salt Form C. FIG. 4C depicts a solid state ²³Na NMR spectrum for Compound 1 Na salt Form C. Table 8 recites ²³Na ssNMR chemical shift data for Compound 1 Na salt Form C.

TABLE 7
¹³C ssNMR chemical shift data of Compound 1 Na salt Form C

| | Chem Shift | |
|---|---|---|
| Peak No. | [ppm] | Intensity [rel] |
| 1 | 173.7 | 11.04 |
| 2 | 172.3 | 13.39 |
| 3 | 163.3 | 11.84 |
| 4 | 160.8 | 18.38 |
| 5 | 145.0 | 14.84 |
| 6 | 144.5 | 15.21 |
| 7 | 139.1 | 21.47 |
| 8 | 138.1 | 52.63 |
| 9 | 137.5 | 48.37 |
| 10 | 137.1 | 48.33 |
| 11 | 134.5 | 33.17 |
| 12 | 132.8 | 33.26 |
| 13 | 132.0 | 40.29 |
| 14 | 131.4 | 46.44 |
| 15 | 129.9 | 86.01 |
| 16 | 128.5 | 48.05 |
| 17 | 122.3 | 22.94 |
| 18 | 121.5 | 23.96 |
| 19 | 118.8 | 19.21 |
| 20 | 117.4 | 23.27 |
| 21 | 115.2 | 27.5 |
| 22 | 114.7 | 32.1 |
| 23 | 113.5 | 14.2 |
| 24 | 103.4 | 15.8 |
| 25 | 99.6 | 26.9 |
| 26 | 98.6 | 27.7 |
| 27 | 73.0 | 31.6 |
| 28 | 72.4 | 38.8 |
| 29 | 70.9 | 70.2 |
| 30 | 70.2 | 85.8 |
| 31 | 68.5 | 100.0 |
| 32 | 67.9 | 72.0 |
| 33 | 65.4 | 9.4 |
| 34 | 61.6 | 22.7 |
| 35 | 60.3 | 24.3 |
| 36 | 36.7 | 29.9 |
| 37 | 35.7 | 46.06 |
| 38 | 32.1 | 59.3 |
| 39 | 31.3 | 22.9 |
| 40 | 29.8 | 6.93 |
| 41 | 27.8 | 10.99 |

TABLE 8
²³Na ssNMR chemical shift data of Compound 1 Na salt Form C

| | Chem Shift | |
|---|---|---|
| Peak No. | [ppm] | Intensity [rel] |
| 1 | −11.2 | 10 |
| 2 | −14.0 | 9.39 |

Thermogravimetric Analysis (TGA): TGA of Compound 1 Na salt Form C was measured using TA Discovery TGA from TA Instrument. A sample with weight of approximately 1-10 mg was scanned from 25° C. to 370° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 4D showed 2.7% weight loss from ambient temperature up to 200° C.

Differential Scanning Calorimetry Analysis (DSC): DSC of Compound 1 Na salt Form C was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram of FIG. 4E showed endothermic peaks at about 47, 66, 81, 176 and 292° C.

5. Compound 1 Na Salt Form D

Synthetic Procedure: ˜40 mg Compound 1 neat Form A was weighed into a 4 mL vial, followed by addition of 870 mg DI water and 88 μl NaOH 1 N solution. The sample was stirred in the cold room at 5° C. for 2 days. Then the solid was collected via centrifugal filtering for further analysis.

X-Ray Powder Diffraction (XRPD): XRPD spectra were recorded at room temperature in transmission mode using a PANalytical Empyrean system equipped with a sealed tube source and a PIXcel 1D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 2θ with a step size of 0.0131303° and 49 s per step. FIG. 5A depicts an XRPD diffractogram of Compound 1 Na salt Form D. Table 9 provides the XRPD peaks, angles and intensity % for Compound 1 Na salt Form D.

TABLE 9
XRPD peaks, angles and intensity
% of Compound 1 Na salt Form D

| | Pos. | |
|---|---|---|
| Peak No. | [±0.2 °2θ] | Rel. Int. [%] |
| 1 | 18.7 | 100.0 |
| 2 | 17.5 | 42.7 |
| 3 | 3.5 | 41.1 |
| 4 | 21.8 | 27.4 |
| 5 | 13.7 | 27.1 |
| 6 | 17.2 | 26.1 |
| 7 | 21.3 | 18.1 |
| 8 | 22.7 | 16.3 |
| 9 | 19.3 | 14.2 |
| 10 | 28.8 | 12.7 |
| 11 | 30.9 | 12.1 |
| 12 | 16.2 | 12.0 |
| 13 | 20.0 | 11.9 |
| 14 | 14.0 | 10.6 |

Solid State NMR (ssNMR): FIG. 5B depicts a solid state ¹³C NMR spectrum for Compound 1 Na salt Form D. Table 10 recites ¹³C ssNMR chemical shift data for Compound 1 Na salt Form D. FIG. 5C depicts a solid state ²³Na NMR spectrum for Compound 1 Na salt Form D. Table 11 recites ²³Na ssNMR chemical shift data for Compound 1 Na salt Form D.

TABLE 10
¹³C ssNMR chemical shift data of Compound 1 Na salt Form D

| | Chem Shift | |
|---|---|---|
| Peak No. | [ppm] | Intensity [rel] |
| 1 | 175.8 | 6.2 |
| 2 | 164.0 | 5.7 |
| 3 | 161.0 | 8.5 |
| 4 | 142.0 | 26.1 |
| 5 | 137.0 | 48.4 |
| 6 | 136.4 | 42.9 |
| 7 | 134.0 | 54.9 |
| 8 | 132.5 | 22.4 |
| 9 | 131.3 | 100.0 |
| 10 | 129.8 | 40.4 |
| 11 | 128.2 | 12.7 |
| 12 | 122.5 | 28.4 |
| 13 | 119.3 | 31.1 |
| 14 | 114.4 | 46.1 |
| 15 | 97.9 | 55.4 |
| 16 | 67.7 | 48.3 |
| 17 | 66.6 | 43.0 |
| 18 | 37.2 | 45.3 |
| 19 | 35.9 | 51.1 |
| 20 | 27.8 | 31.8 |

TABLE 11
²³Na ssNMR chemical shift data of Compound 1 Na salt Form D

| | Chem Shift | |
|---|---|---|
| Peak No. | [ppm] | Intensity [rel] |
| 1 | 5.3 | 2.41 |
| 2 | 2.1 | 3.88 |
| 3 | −5.0 | 9.89 |
| 4 | −6.3 | 10 |

6. Compound 1 Ca Salt Form A

Synthetic Procedure: Compound 1 Ca salt Form A was prepared via reacting 20 mg Compound 1 neat Form A with 1.4 mg Ca(OH)₂ in 0.3 mL TH/water (9:1, v/v) at RT stirring for 2-4 days (i.e., molar charge ratio of free form/counter ion at 2:1). The solids were filtered and air dried before the further analysis.

X-Ray Powder Diffraction (XRPD): FIG. 6A depicts an XRPD diffractogram of Compound 1 Ca salt Form A. Table 12 provides the XRPD peaks, angles, and intensity % for Compound 1 Ca salt Form A.

TABLE 12
XRPD peaks, angles and intensity
% of Compound 1 Ca salt Form A

| | Pos. | |
|---|---|---|
| Peak No. | [±0.2 °2θ] | Rel. Int. [%] |
| 1 | 17.9 | 100.0 |
| 2 | 11.7 | 85.6 |
| 3 | 20.5 | 35.1 |
| 4 | 20.9 | 34.6 |
| 5 | 22.0 | 30.3 |
| 6 | 7.3 | 29.9 |
| 7 | 9.9 | 29.2 |
| 8 | 12.4 | 22.5 |
| 9 | 19.2 | 21.9 |
| 10 | 5.2 | 18.5 |
| 11 | 14.5 | 15.6 |
| 12 | 18.6 | 12.9 |
| 13 | 24.7 | 11.3 |
| 14 | 16.4 | 11.2 |
| 15 | 24.1 | 11.0 |
| 16 | 23.5 | 10.7 |
| 17 | 10.6 | 10.4 |

Thermogravimetric Analysis (TGA): TGA of Compound 1 Ca salt Form A was measured using TA Discovery 550 TGA from TA Instrument. A sample with weight of approximately 1-5 mg was scanned from 25° C. to 375° C. at a heating rate of 10° C./min with nitrogen purge. Data were collected by Thermal Advantage Q Series' software and analyzed by Trios and/or Universal Analysis software (TA Instruments, New Castle, DE). The thermogram of FIG. 6B showed 15.6% weight loss from ambient temperature up to 250° C.

Differential Scanning Calorimetry Analysis (DSC): DSC of Compound 1 Ca salt Form A was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 375° C. When the run was completed, the data were analyzed by Trios and/or Universal Analysis software (TA Instruments, New Castle, DE). The thermogram of FIG. 6C showed endothermic peaks at about 140, 200, and 250° C.

7. Compound 1 HCl Salt Form A

Synthetic Procedure: Compound 1 HCl salt Form A was prepared via slurry of 20 mg Compound 1 neat Form A with 4.33 mg HCl in 2 mL ACN at RT for 2-4 days (i.e., molar ratio of free form/counter ion at 1:1). The solids were filtered and air dried before the further analysis.

X-Ray Powder Diffraction (XRPD): FIG. 7A depicts an XRPD diffractogram of Compound 1 HCl salt Form A. Table 13 provides the XRPD peaks, angles, and intensity % for Compound 1 HCl salt Form A.

TABLE 13
XRPD peaks, angles, and intensity
% of Compound 1 HCl salt Form A

| | Pos. | |
|---|---|---|
| Peak No. | [±0.2 °2θ] | Rel. Int. [%] |
| 1 | 8.1 | 100.0 |
| 2 | 7.8 | 87.8 |
| 3 | 9.0 | 34.5 |
| 4 | 19.8 | 30.7 |
| 5 | 12.2 | 16.8 |
| 6 | 20.1 | 15.6 |
| 7 | 17.2 | 11.6 |
| 8 | 23.8 | 10.6 |

Thermogravimetric Analysis (TGA): TGA of Compound 1 HCl salt Form A was measured using TA Discovery 550 TGA from TA Instrument. A sample with weight of approximately 1-5 mg was scanned from 25° C. to 375° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 7B showed 6.9% weight loss from ambient temperature up to 250° C.

Differential Scanning Calorimetry Analysis (DSC): DSC of Compound 1 HCl salt Form A was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 375° C. When the run was completed, the data were analyzed by Trios and/or Universal Analysis software (TA Instruments, New Castle, DE). The thermogram of FIG. 7C showed endothermic peaks at about 208 and 328° C.

8. Compound 1 DMSO Solvate Form A

Synthetic Procedure: ˜20 mg of Compound 1 neat Form A was suspended in 0.3 mL DMSO in a 2-mL glass vial. After the suspension was stirred magnetically for two days at 100° C., the remaining solids were isolated for analysis.

X-Ray Powder Diffraction (XRPD): FIG. 8A depicts an XRPD diffractogram of Compound 1 DMSO solvate Form A. Table 14 provides the XRPD peaks, angles and intensity % for Compound 1 DMSO solvate Form A.

TABLE 14
XRPD peaks, angles and intensity %
of Compound 1 DMSO solvate Form A

| | Pos. | |
|---|---|---|
| Peak No. | [±0.2 °2θ] | Rel. Int. [%] |
| 1 | 19.8 | 100.0 |
| 2 | 9.9 | 87.3 |
| 3 | 19.1 | 73.7 |
| 4 | 20.7 | 53.9 |
| 5 | 11.0 | 28.7 |
| 6 | 4.9 | 20.0 |
| 7 | 14.8 | 15.8 |
| 8 | 7.1 | 14.5 |

Thermogravimetric Analysis (TGA): TGA of Compound 1 DMSO solvate Form A was measured using TA Discovery 550 TGA from TA Instrument. A sample with weight of approximately 1-5 mg was scanned from 25° C. to 290° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 8B showed minimal weight loss from ambient temperature up to 200° C. and had 14% weight loss between 200-250° C.

Differential Scanning Calorimetry Analysis (DSC): DSC of Compound 1 DMSO solvate Form A was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 300° C. When the run was completed, the data were analyzed by Trios and/or Universal Analysis software (TA Instruments, New Castle, DE). The thermogram of FIG. 8C showed endothermic peaks at about 100, 155, and 257° C.

9. Compound 1 EtOH Solvate Form A

Synthetic Procedure: Compound 1 was dissolved in THF:H₂O (9:1) at 60° C. Water was added to precipitate Compound 1 followed by mixing for 1 hour. The solid was collected by filtration, and the cake was resuspended in EtOH via mixing for 30 minutes. The solid was collected again by filtration and dried under vacuum for 18 hours at 66° C.

X-Ray Powder Diffraction (XRPD): XRPD spectra were recorded at room temperature in reflection mode using a PANalytical Empyrean system equipped with a sealed tube source and a PIXcel 1D Medipix-2 detector (Malvern PANalytical Inc, Westborough, Massachusetts). The X-Ray generator operated at a voltage of 45 kV and a current of 40 mA with copper radiation (1.54060 Å). The powder sample was placed in a back filled sample holder and loaded into the instrument. The sample was scanned over the range of about 3° to about 40° 2θ with a step size of 0.0131303° and 49.725 s per step. FIG. 9A depicts an XRPD diffractogram of Compound 1 EtOH solvate Form A. Table 15 provides the XRPD peaks, angles and intensity % for Compound 1 EtOH solvate Form A.

TABLE 15
XRPD peaks, angles and intensity %
of Compound 1 EtOH solvate Form A

| | Pos. | |
|---|---|---|
| Peak No. | [±0.2 °2θ] | Rel. Int. [%] |
| 1 | 20.7 | 100.0 |
| 2 | 23.4 | 99.0 |
| 3 | 20.2 | 88.0 |
| 4 | 21.0 | 76.5 |
| 5 | 17.1 | 62.7 |
| 6 | 18.2 | 53.5 |
| 7 | 12.0 | 45.6 |
| 8 | 19.8 | 44.0 |
| 9 | 28.6 | 43.2 |
| 10 | 18.9 | 41.3 |
| 11 | 22.9 | 36.0 |
| 12 | 13.8 | 27.1 |
| 13 | 7.5 | 24.2 |
| 14 | 15.9 | 21.4 |
| 15 | 26.7 | 19.5 |
| 16 | 16.6 | 19.3 |
| 17 | 29.2 | 18.5 |
| 18 | 21.4 | 16.1 |
| 19 | 26.4 | 16.0 |
| 20 | 12.6 | 14.8 |
| 21 | 22.4 | 14.7 |
| 22 | 24.6 | 14.3 |
| 23 | 29.6 | 12.4 |

Solid State NMR (ssNMR): FIG. 9B depicts a solid state ¹³C NMR spectrum for Compound 1 EtOH solvate Form A. Table 16 recites ¹³C ssNMR chemical shift data for Compound 1 EtOH solvate Form A.

TABLE 16
¹³C ssNMR chemical shift data of
Compound 1 EtOH solvate Form A

| | Chem Shift | |
|---|---|---|
| Peak No. | [ppm] | Intensity [rel] |
| 1 | 172.3 | 26.79 |
| 2 | 162.9 | 4.09 |
| 3 | 161.2 | 5.37 |
| 4 | 144.7 | 19.98 |
| 5 | 141.7 | 23.35 |
| 6 | 137.7 | 27.78 |
| 7 | 134.9 | 26.65 |
| 8 | 131.1 | 100 |
| 9 | 128.6 | 30 |
| 10 | 126.6 | 25.17 |
| 11 | 121.1 | 23.58 |
| 12 | 117.4 | 37.94 |
| 13 | 111.5 | 18.37 |
| 14 | 99.2 | 25.99 |
| 15 | 95.8 | 27.74 |
| 16 | 68.1 | 52.69 |
| 17 | 57.9 | 36.15 |
| 18 | 36.1 | 37.7 |
| 19 | 34.4 | 29.06 |
| 20 | 27.9 | 26.3 |
| 21 | 19.0 | 32.15 |

Thermogravimetric Analysis (TGA): TGA of Compound 1 EtOH solvate Form A was measured using TA Instruments TGA Q5000. A sample with weight of approximately 1-10 mg was scanned from 25° C. to 250° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 9C showed 9.0% weight loss from ambient temperature up to 200° C.

Differential Scanning Calorimetry Analysis (DSC): DSC of Compound 1 EtOH solvate Form A was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 375° C. When the run was completed, the data were analyzed by Trios and/or Universal Analysis software (TA Instruments, New Castle, DE). The thermogram of FIG. 9D showed endothermic peaks at about 116, 140, and 350° C.

10. Compound 1 Tartrate Salt Cocrystal Form A

Synthetic Procedure: 9.5 mg Compound 1 neat Form A and 22 μl 1 M NaOH were mixed first, and 3.2 mg tartaric acid and 0.5 mL THF/water (9:1, v:v) were added in (molar charge ratio of 1:1:1 for neat form/base/acid) during stirring at room temperature. The mixture was solubilized at 60° C., followed by stirring at room temperature and evaporating the solution to obtain the product.

X-Ray Powder Diffraction (XRPD): FIG. 10A depicts an XRPD diffractogram of Compound 1 tartrate salt or cocrystal Form A. Table 17 provides the XRPD peaks, angles, and intensity % for Compound 1 tartrate salt or cocrystal Form A.

TABLE 17
XRPD peaks, angles and intensity % of Compound
1 tartrate salt or cocrystal Form A

| | Pos. | |
|---|---|---|
| Peak No. | [±0.2 °2θ] | Rel. Int. [%] |
| 1 | 20.5 | 100.0 |
| 2 | 19.6 | 38.5 |
| 3 | 26.5 | 36.4 |
| 4 | 19.0 | 27.4 |
| 5 | 26.6 | 18.7 |
| 6 | 19.4 | 14.8 |
| 7 | 22.1 | 11.5 |

Thermogravimetric Analysis (TGA): TGA of Compound 1 tartrate salt or cocrystal Form A was measured using TA Discovery 550 TGA from TA Instrument. A sample with weight of approximately 1-5 mg was scanned from 25° C. to 280° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 10B showed 3.9% weight loss from ambient temperature up to 200° C.

Differential Scanning Calorimetry Analysis (DSC): DSC of Compound 1 tartrate salt or cocrystal Form A was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 295° C. When the run was completed, the data were analyzed by Trios and/or Universal Analysis software (TA Instruments, New Castle, DE). The thermogram of FIG. 10C showed endothermic peaks at about 120 and 250° C.

11. Compound 1 Tartrate Salt or Cocrystal Form B

Synthetic Procedure: 9.9 mg Compound 1 neat Form A and 0.8 mg Ca(OH)₂ were weighed first, and 3.2 mg tartaric acid and 0.5 mL EtOAc were added in (molar charge ratio of 2:1:1 for neat form/base/acid) during stirring at room temperature. The mixture was solubilized at 60° C., followed by stirring at room temperature and evaporating the solution to obtain the product.

X-Ray Powder Diffraction (XRPD): FIG. 11A depicts an XRPD diffractogram of Compound 1 salt or cocrystal Form B. Table 18 provides the XRPD peaks, angles, and intensity % for Compound 1 tartrate salt or cocrystal Form B.

TABLE 18
XRPD peaks, angles and intensity % of Compound
1 tartrate salt or cocrystal Form B

| | Pos. | |
|---|---|---|
| Peak No. | [±0.2 °2θ] | Rel. Int. [%] |
| 1 | 20.8 | 100.0 |
| 2 | 19.3 | 79.1 |
| 3 | 12.9 | 70.3 |
| 4 | 22.7 | 70.0 |
| 5 | 8.9 | 54.9 |
| 6 | 17.8 | 38.2 |
| 7 | 20.3 | 31.5 |
| 8 | 18.2 | 26.5 |
| 9 | 21.7 | 26.3 |
| 10 | 16.8 | 26.2 |
| 11 | 22.3 | 22.6 |
| 12 | 6.6 | 22.2 |
| 13 | 22.0 | 20.8 |
| 14 | 20.1 | 20.5 |
| 15 | 29.5 | 18.7 |
| 16 | 26.0 | 18.0 |
| 17 | 26.5 | 16.0 |
| 18 | 19.8 | 14.7 |
| 19 | 24.7 | 14.5 |
| 20 | 11.9 | 11.3 |
| 21 | 18.8 | 10.6 |
| 22 | 23.6 | 10.6 |

Thermogravimetric Analysis (TGA): TGA of Compound 1 tartrate salt or cocrystal Form B was measured using TA Discovery 550 TGA from TA Instrument. A sample with weight of approximately 1-5 mg was scanned from 25° C. to 280° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 11B showed 10.8% weight loss from ambient temperature up to 200° C.

Differential Scanning Calorimetry Analysis (DSC): DSC of Compound 1 tartrate salt or cocrystal Form B was measured using the TA Q2000 DSC from TA Instrument. A sample with a weight between 1-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 295° C. When the run was completed, the data were analyzed by Trios and/or Universal Analysis software (TA Instruments, New Castle, DE). The thermogram of FIG. 11C showed the endothermic peak around 180° C. and exothermic peak around 182° C.

12. Compound 1 Tartrate Salt or Cocrystal Form C

Synthetic Procedure: 10.3 mg Compound 1 neat Form A and 0.8 mg Ca(OH)₂ were weighed first, and 3.4 mg tartaric acid and 0.5 mL THF/H₂O (9:1, v:v) were added in (molar charge ratio of 2:1:1 for neat form/base/acid) during stirring at room temperature. The mixture was solubilized at 60° C., followed by stirring at room temperature and evaporating the solution to obtain the product.

X-Ray Powder Diffraction (XRPD): FIG. 12A depicts an XRPD diffractogram of Compound 1 tartrate salt or cocrystal Form C. Table 19 provides the XRPD peaks, angles and intensity % for Compound 1 tartrate salt or cocrystal Form C.

TABLE 19
XRPD peaks, angles and intensity % of Compound
1 tartrate salt or cocrystal Form C

| | Pos. | |
|---|---|---|
| Peak No. | [±0.2 °2θ] | Rel. Int. [%] |
| 1 | 13.3 | 100.0 |
| 2 | 12.4 | 87.9 |
| 3 | 18.5 | 65.9 |
| 4 | 29.5 | 49.4 |
| 5 | 29.2 | 45.7 |
| 6 | 16.8 | 45.2 |
| 7 | 21.5 | 41.8 |
| 8 | 19.4 | 25.8 |
| 9 | 27.1 | 16.6 |
| 10 | 22.5 | 15.3 |
| 11 | 15.8 | 6.9 |

Thermogravimetric Analysis (TGA): TGA of Compound 1 tartrate salt or cocrystal Form C was measured using TA Discovery 550 TGA from TA Instrument. A sample with weight of approximately 1-5 mg was scanned from 25° C. to 280° C. at a heating rate of 10° C./min with nitrogen purge. Data were collected by Thermal Advantage Q Series™ software and analyzed by Trios and/or Universal Analysis software (TA Instruments, New Castle, DE). The thermogram of FIG. 12B showed 19% weight loss from ambient temperature up to 200° C.

Differential Scanning Calorimetry Analysis (DSC): DSC of Compound 1 tartrate salt or cocrystal Form C was measured using the TA Q2000 DSC from TA Instrument. A sample with a weight between 0.5-5 mg was weighed into an aluminum pan and crimped. This pan was placed in the sample position in the calorimeter cell. An empty pan was placed in the reference position. The calorimeter cell was closed and a flow of nitrogen was passed through the cell. The heating program was set to heat the sample at a heating rate of 10° C./min to a temperature of 295° C. When the run was completed, the data were analyzed by Trios and/or Universal Analysis software (TA Instruments, New Castle, DE). The thermogram of FIG. 12C showed endothermic peaks around 119 and 142° C.

13. Compound 1 Tartrate Salt or Cocrystal Form D

Synthetic Procedure: 9.7 mg Compound 1 neat Form A and 0.6 mg Mg(OH)₂ were weighed first, and 3.4 mg tartaric acid and 0.5 mL THF/H₂O (9:1, v:v) were added in (molar charge ratio of 2:1:1 for neat form/base/acid) during stirring at room temperature. The mixture was solubilized at 60° C., followed by stirring at room temperature and evaporating the solution to obtain the product.

X-Ray Powder Diffraction (XRPD): FIG. 13 depicts an XRPD diffractogram of Compound 1 tartrate salt or cocrystal Form D. Table 20 provides the XRPD peaks, angles and intensity % for Compound 1 tartrate salt or cocrystal Form D.

TABLE 20
XRPD peaks, angles and intensity % of Compound
1 tartrate salt or cocrystal Form D

| | Pos. | |
|---|---|---|
| Peak No. | [±0.2 °2θ] | Rel. Int. [%] |
| 1 | 13.8 | 100.0 |
| 2 | 16.8 | 91.1 |
| 3 | 14.8 | 68.4 |
| 4 | 23.9 | 40.8 |
| 5 | 25.2 | 29.5 |
| 6 | 27.7 | 22.2 |
| 7 | 21.9 | 20.9 |
| 8 | 24.5 | 19.4 |
| 9 | 28.3 | 19.3 |
| 10 | 19.5 | 19.1 |
| 11 | 18.7 | 16.8 |
| 12 | 12.5 | 14.5 |
| 13 | 22.5 | 11.4 |

### Example 3. Solid Dispersions of Compound 1

Various spray dried dispersions (SDD) of Compound 1 were prepared using either 50% or 80% drug loading (DL) and with different polymers (e.g., HPMCAS-H, PVPVA), different organic solvent systems (e.g., DCM, MeOH, EtOH, THF, Me-THF) and at different amounts of water at specific weight or volume ratios. Without wishing to be bound by theory, the inventors have found that the SDD of Compound 1 prepared using the solvent ratios as described herein lead to improved solubility and stability of the drug in the dispersion and/or more desirable spray drying process space, whereby a broader range of feed rates (e.g., 15-45 kg/h vs. about 20-34 kg/h) may be explored. The benefit of a broader range of feed rates during the spray drying process is that it allows the inventors to determine whether there are any changes to various material properties of the SDD (e.g., particle size, powder density, surface morphology, crystallinity) as the process for making the SDD's are being scaled up.

1. Compound 1 50% DL Amorphous Spray Dried Dispersion [DCM/MeOH at 80/20 v with HPMCAS-H]

Synthesis Procedure. 10 g of Compound 1 was weighed into a bottle. 500 mL of 80/20 v/v DCM/MeOH was added. The bottle was capped and the contents were stirred at ambient temperature until a clear solution resulted. 10 g of hydroxypropylmethylcellulose acetate succinate H grade (HPMCAS-H) was added. The bottle was capped and the contents were stirred for ˜1 h at ambient temperature until a clear solution resulted. This solution was then spray dried to make amorphous Compound 1.

2. Compound 1 50% DL Amorphous Spray Dried Dispersion [DCM/EtOH at 60/40 v/v with HPMCAS-H]

Synthesis Procedure. 30 g of Compound 1 was weighed into a bottle. 1000 mL of 60/40 v/v DCM/EtOH was added. The bottle was capped and the contents were stirred for 0.5 h at ambient temperature when a clear solution resulted. 30 g of hydroxypropylmethylcellulose acetate succinate H grade (HPMCAS-H) was added. The bottle was capped and the contents were stirred for˜1 h at ambient temperature when a clear solution resulted. This solution was then spray dried to make amorphous Compound 1.

3. Compound 1 80% DL Amorphous Spray Dried Dispersion [THF/MeOH/H₂O at 75/15/10 w/w with HPMCAS-H]

Synthesis Procedure. 1.2 g of Compound 1 was weighed into a bottle. 17.3 g of 75/15/10 w/w THF/MeOH/water was added. The bottle was capped and the contents were stirred at ambient temperature until a clear solution resulted. 0.3 g of hydroxypropylmethylcellulose acetate succinate H grade (HPMCAS-H) was added. The bottle was capped and the contents were stirred at ambient temperature until a clear solution resulted. This solution was then spray dried to make amorphous Compound 1.

4. Compound 1 80% DL Amorphous Spray Dried Dispersion [DCM/EtOH/H₂O at 56.8/33.79.5 w/w with PVP-VA]

Synthesis Procedure. 8 g of Compound 1 was weighed into a bottle. 156.7 g of 56.8/33.7/9/5 w/w DCM/EtOH/water was added. The bottle was capped and the contents were stirred for ˜1 h at ambient temperature when a clear solution resulted. 2 g of polyvinylpyrrolidone-vinyl acetate (PVP-VA) was added. The bottle was capped and the contents were stirred at ambient temperature until a clear solution resulted. This solution was then spray dried to make amorphous Compound 1.

5. Compound 1 50% DL Amorphous Spray Dried Dispersion [DCM/MeOH/H₂O at 67.8/31.3/0.9 w/w with HPMCAS-H]

Synthesis Procedure. 359 g of Compound 1 was weighed into a bottle. 11688 g of 67.8/31.3/0.9 w/w DCM/MeOH/H₂O was added. The bottle was capped and the contents were stirred at ambient temperature until a clear solution resulted. 359 g of hydroxypropylmethylcellulose acetate succinate H grade (HPMCAS-H) was added. The bottle was capped and the contents were stirred at ambient temperature until a clear solution resulted. This solution was then spray dried to make amorphous Compound 1.

6. Compound 1 80% DL Amorphous Spray Dried Dispersion [DCM/MeOH/H₂O at 56.8/3.79.5 w/w with HPMCAS-H]

Synthesis Procedure. 8020.8 g of 56.8/33.7/9.5 w/w DCM/MeOH/H₂O was weighed into a suitably sized bottle. 250 g of Compound 1 was added to the bottle. The bottle was capped and the contents were stirred at ambient temperature until a clear solution resulted. 62.5 g of hydroxypropylmethylcellulose acetate succinate H grade (HPMCAS-H) was added. The bottle was capped and the contents were stirred at ambient temperature until a clear solution resulted. This solution was then spray dried to make amorphous Compound 1.

### Example 4. AAT Modulators Compounds 2 and 3

### Preparation of Compound 2

### 4-[1-(2-cyano-1,1-dimethyl-ethyl)-10-(3,4-difluorophenyl)-2,4,10-triazatricyclo[7.3.0.03,7]dodeca-1,3(7),5,8,11-pentaen-12-yl]benzoic acid (Compound 2)

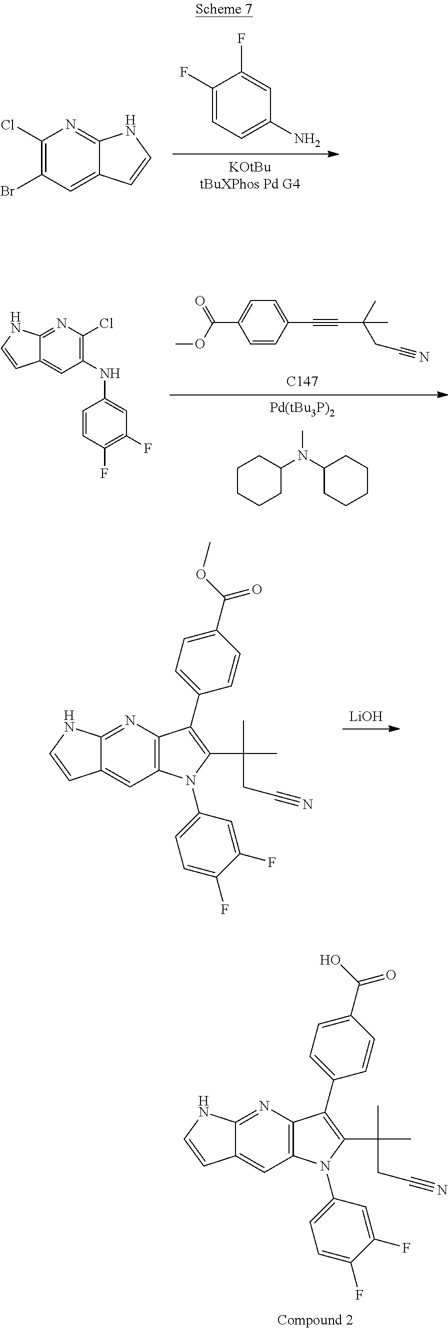

### Step 1. Synthesis of 6-chloro-N-(3,4-difluorophenyl)-1H-pyrrolo[2,3-b]pyridin-5-amine

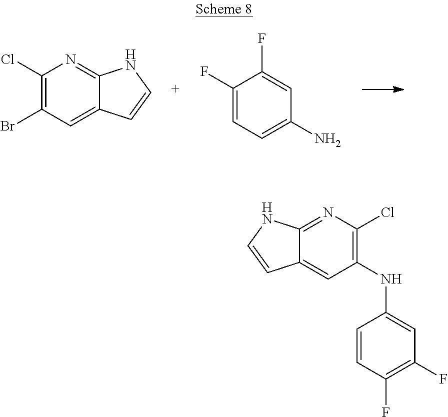 [SMILES: Clc1nc2[nH]ccc2cc1Br ; Nc1ccc(F)c(F)c1 ; Fc1ccc(Nc2cc3cc[nH]c3nc2Cl)cc1F]

Weighed 5-bromo-6-chloro-1H-pyrrolo[2,3-b]pyridine (944.5 mg, 4.080 mmol), t-butyl XPhos Palladacycle generation 4 (362 mg, 0.4052 mmol), potassium t-butoxide (1.372 g, 12.23 mmol), and ditert-butyl-[2-(2,4,6-triisopropylphenyl)phenyl] phosphane (362 mg, 0.8525 mmol) into a 40 mL vial. Added t-butanol (21 mL) and warmed in a 30° C. heating block. Degassed with N2 for 1 minute. Added 3,4-difluoroaniline (425 μL, 4.286 mmol) and stirred at 30° C. overnight. Diluted with dichloromethane (100 mL). Washed the organics with 0.5 M HCl aqueous solution (50 mL), and washed the aqueous phase with 10% methanol in dichloromethane (100 mL). Combined the organic phases and dried over Na₂SO₄, filtered, added celite and evaporated the solvent under reduced pressure. Purification by silica gel chromatography on 80 g of Silica in a gold cartridge: (Gradient: 0-100% ethyl acetate in heptane). Concentrated the desired fractions to dryness under reduced pressure to yield the product as a white solid, 6-chloro-N-(3,4-difluorophenyl)-1H-pyrrolo[2,3-b]pyridin-5-amine (620 mg, 53%) 1H NMR (300 MHz, DMSO-d6) δ 11.82 (s, 1H), 7.95 (s, 1H), 7.84 (s, 1H), 7.55-7.46 (m, 1H), 7.24-7.11 (m, 1H), 6.63 (ddd, J=13.2, 7.0, 2.8 Hz, 1H), 6.53-6.41 (m, 2H). ESI-MS m/z calc. 279.03748, found 280.2 (M+1)⁺; Retention time: 0.83 minutes. Final purity was determined by reversed phase UPLC using an Acquity UPLC Acquity CSH C18 (2.1×50 mm, 1.7 μm particle) made by Waters, and a dual gradient run from 5-95% mobile phase B over 0.6 minutes. Mobile phase A=H₂O (0.1% CF₃CO₂H). Mobile phase B=CH₃CN (0.1% CF₃CO₂H). Flow rate=0.6 mL/min, injection volume=2.0 μL.

### Step 2. Synthesis of 4-[11-(2-cyano-1,1-dimethyl-ethyl)-10-(3,4-difluorophenyl)-2,4,10-triazatricyclo[7.3.0.03,7]dodeca-1,3(7),5,8,11-pentaen-12-yl]benzoic acid

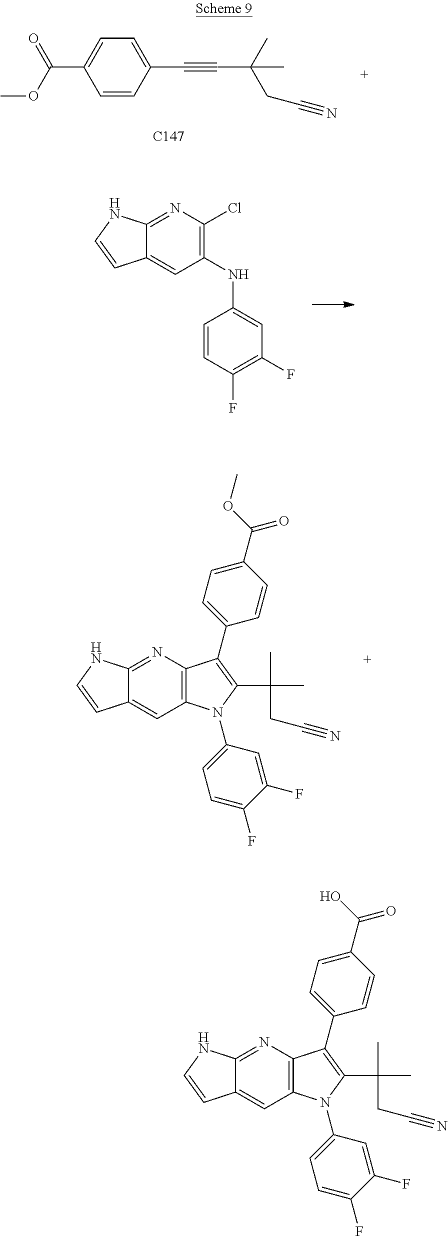 [SMILES: COC(=O)c1ccc(C#CC(C)(C)CC#N)cc1 ; "C147" ; Fc1ccc(Nc2cc3cc[nH]c3nc2Cl)cc1F ; COC(=O)c1ccc(-c2c(C(C)(C)CC#N)n(-c3ccc(F)c(F)c3)c3cc4cc[nH]c4nc23)cc1 ; CC(C)(CC#N)c1c(-c2ccc(C(=O)O)cc2)c2nc3[nH]ccc3cc2n1-c1ccc(F)c(F)c1]

6-chloro-N-(3,4-difluorophenyl)-1H-pyrrolo[2,3-b]pyridin-5-amine (49.9 mg, 0.1756 mmol) and methyl 4-(4-cyano-3,3-dimethyl-but-1-ynyl)benzoate (approximately 69.3 mg, 0.273 mmol) were dissolved in 1,4-dioxane (1 mL) and N-cyclohexyl-N-methyl-cyclohexanamine (101.6 mg, 111.4 μL, 0.5201 mmol). The solution was degassed with N2 for 10 minutes, followed by addition of Bis(tri-tert-butylphosphine)palladium(0) (8.9 mg, 0.0175 mmol). The reaction was heated to 80° C. After 18 hours, the reaction was complete. The reaction was allowed to cool to room temperature and concentrated to dryness under reduced pressure. Directly added to the crude, methanol (2 mL), THF (2 mL), and LiOH (1 mL of 2 M, 2.0 mmol). Stirred at 50° C. for 2 hours. Concentrated the mixture to dryness under reduced pressure. Added dimethyl sulfoxide, 3 mL. Injected on a C18 RP Column (50 g): Purification by reversed-phase chromatography (Column: C18. Gradient: 10-100% Acetonitrile in water with 0.1% formic acid) afforded the desired product in insufficient purity. Combined desired fractions and concentrated to dryness under reduced pressure. Diluted with dichloromethane (3 mL) and a few drops of methanol and purified on a normal phase silica cartridge, 24 g. Silica Gradient: Purification by silica gel chromatography (Gradient: 0-10% methanol in dichloromethane) yielded the product. Concentrated the desired fractions to dryness under reduced pressure to give 4-[11-(2-cyano-1,1-dimethyl-ethyl)-10-(3,4-difluorophenyl)-2,4,10-triazatricyclo[7.3.0.03,7]dodeca-1,3(7),5,8,11-pentaen-12-yl]benzoic acid (8.0 mg, 9%) ¹H NMR (300 MHz, DMSO-d6) δ 12.99 (s, 1H), 11.31 (s, 1H), 8.05 (d, J=8.0 Hz, 2H), 7.87 (t, J=9.4 Hz, 1H), 7.75 (q, J=9.3 Hz, 1H), 7.60 (d, J=8.0 Hz, 2H), 7.54-7.43 (m, 1H), 7.37 (t, J=2.9 Hz, 1H), 7.23 (s, 1H), 6.39-6.30 (m, 11H), 2.66 (s, 2H), 1.27 (d, J=4.8 Hz, 6H). ESI-MS m/z calc. 470.15543, found 471.47 (M+1)⁺; Retention time: 0.67 minutes Final purity was determined by reversed phase UPLC using an Acquity UPLC Acquity CSH C18 (2.1×50 mm, 1.7 μm particle) made by Waters, and a dual gradient run from 5-95% mobile phase B over 0.6 minutes. Mobile phase A=H₂O (0.1% CF₃CO₂H). Mobile phase B=CH₃CN (0.1% CF₃CO₂H). Flow rate=0.6 mL/min, injection volume=2.0 μL.

Processes for preparing Compound 3 comprise reactions depicted in Schemes 10-11 below:

### Preparation of Compound 3

### Preparation of S7

### 1-(S-(4 fluorophenyl)-7-iodo-6-isopropylpyrrolo[2,3 f]indazol-1(5H)-yl)-2,2-dimethylpropan-1-one (S7)

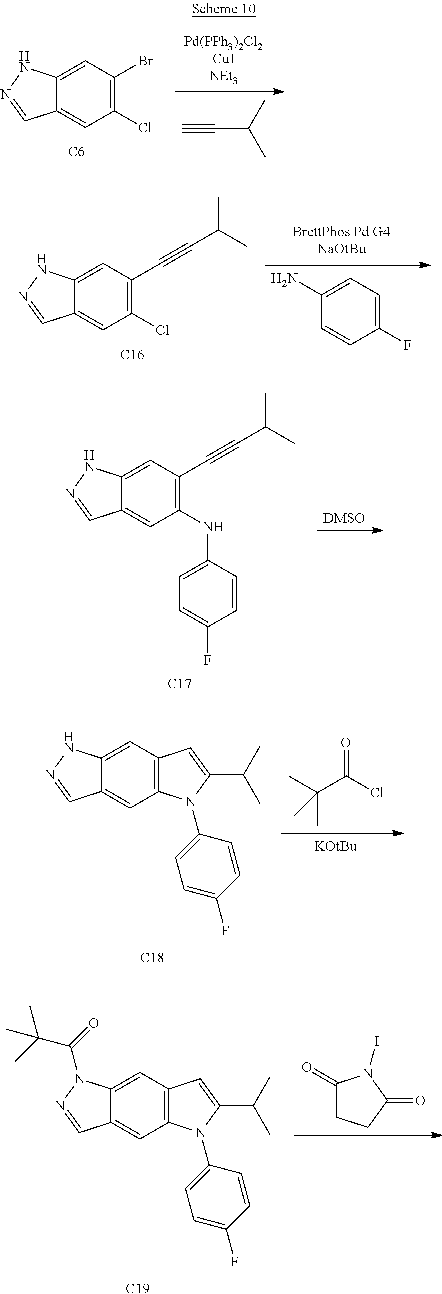

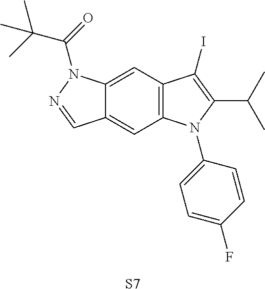 [SMILES: CC(C)c1c(I)c2cc3c(cnn3C(=O)C(C)(C)C)cc2n1-c1ccc(F)cc1 ; "S7"]

### Step 1. Synthesis of 5-chloro-6-(3-methylbut-1-yn-1-yl)-1H-indazole (C16)

Pd(PPh₃)₂Cl₂ (1.7 g, 2.4 mmol) was added to a nitrogen purged solution of 3-methylbut-1-yne (10.7 mL, 104.6 mmol), 6-bromo-5-chloro-1H-indazole C6 (10.4 g, 44.9 mmol) and CuI (497 mg, 2.6 mmol) in Et₃N (100 mL) and 1,4-dioxane (100 mL). The solution was stirred at 90° C. overnight in a Parr bottle, whereupon Celite® and methanol were added, and the mixture concentrated in vacuo. Purification of the Celite® adsorbed mixture by silica gel chromatography (Gradient: 0-100% EtOAc in heptanes) afforded the product (7.0 g, 71%). ¹H NMR (300 MHz, Chloroform-d) δ 10.17 (s, 1H), 8.02 (d, J=1.1 Hz, 1H), 7.80 (d, J=0.7 Hz, 1H), 7.62 (t, J=0.9 Hz, 1H), 2.88 (hept, J=6.9 Hz, 1H), 1.34 (d, J=6.9 Hz, 6H). LCMS m/z 219.04 [M+H]⁺.

### Step 2. Synthesis of N-(4-fluoro-3-methylphenyl)-6-(3-methylbut-1-yn-1-yl)-1H-indazol-5-amine (C17)

t-Butanol (45 mL) and 1,4-dioxane (15 mL) were added to a flask containing 4-fluoro-3-methyl-aniline (2.1 g, 16.8 mmol), 5-chloro-6-(3-methylbut-1-ynyl)-1H-indazole C16 (2.3 g, 10.5 mmol), sodium t-butoxide (3.9 g, 40.6 mmol), and BrettPhos Pd G4 catalyst (280 mg, 0.3 mmol). The mixture was degassed and stirred under N₂ at 100° C. overnight. The mixture was concentrated under reduced pressure, re-dissolved in dichloromethane, and washed with water. The organic layer was dried by passing through a phase separator and concentrated in vacuo. Silica gel chromatography (Gradient: 0-100% EtOAc in heptanes) afforded the product (1.9 g, 58%). ¹H NMR (300 MHz, DMSO-d₆) δ 12.93 (s, 1H), 7.92 (s, 1H), 7.52 (s, 1H), 7.40 (s, 1H), 7.16 (s, 1H), 7.02-6.91 (m, 1H), 6.87-6.71 (m, 2H), 2.75 (m, 1H), 2.15 (d, J=1.9 Hz, 3H), 1.11 (d, J=6.9 Hz, 6H). LCMS m/z 308.2 [M+H]⁺.

### Step 3. Synthesis of 5-(4-fluoro-3-methylphenyl)-6-isopropyl-1,5-dihydropyrrolo[2,3-f]indazole (C18)

A solution of N-(4-fluoro-3-methyl-phenyl)-6-(3-methylbut-1-ynyl)-1H-indazol-5-amine C17 (254 mg, 0.83 mmol) in DMSO (2.3 mL) was heated under microwave conditions at 150° C. for 30 min. The reaction mixture was poured into water (30 mL) and stirred for 4 h. The resulting solid was filtered and dried under vacuum at 50° C. to afford the product (143 mg, 53%). ¹H NMR (300 MHz, DMSO-d₆) δ 12.58 (s, 1H), 7.96 (d, J=1.3 Hz, 1H), 7.53 (d, J=1.1 Hz, 1H), 7.45-7.27 (m, 3H), 7.16 (d, J=1.0 Hz, 1H), 6.46 (d, J=0.9 Hz, 1H), 3.03-2.83 (m, 1H), 2.34 (d, J=2.0 Hz, 3H), 1.18 (d, J=6.8 Hz, 6H). LCMS m/z 308.2 [M+H]⁺.

### Step 4. Synthesis of 1-[5-(4-fluorophenyl)-6-isopropyl-pyrrolo[2,3-f]indazol-1-yl]-2,2-dimethyl-propan-1-one (C19)

A solution of 5-(4-fluorophenyl)-6-isopropyl-1H-pyrrolo[2,3-f]indazole C18 (60 g, 204.5 mmol) in THF (600 mL) was cooled to 0° C. KOtBu (29.8 g, 265.9 mmol) was added and the mixture allowed to stir at 0° C. for 10 min. 2,2-dimethylpropanoyl chloride (34 mL, 276.3 mmol) was added and the mixture allowed to stir at room temperature for 1 h. Saturated NH₄Cl (640 mL) and EtOAc were added. The aqueous layer was isolated and further extracted with EtOAc. Combined organic layers were dried, and concentrated in vacuo. Purification by silica gel chromatography (Column: 1.5 kg silica gel. Gradient: 0-30% EtOAc/Heptane) afforded the product as a yellow solid (64 g, 83%). ¹H NMR (300 MHz, Chloroform-d) δ 8.67 (t, J=0.9 Hz, 1H), 8.05 (d, J=0.8 Hz, 1H), 7.44-7.32 (m, 2H), 7.32-7.26 (m, 2H), 7.19 (t, J=0.9 Hz, 1H), 6.56 (t, J=0.8 Hz, 1H), 3.04-2.88 (m, 1H), 1.60 (s, 9H), 1.26 (d, J=6.8 Hz, 6H). LCMS m/z 378.17 [M+H]⁺.

### Step 5. Synthesis of 1-[5-(4-fluorophenyl)-7-iodo-6-isopropyl-pyrrolo[2,3-f]indazol-1-yl]-2,2-dimethyl-propan-1-one (S7)

To a solution of 1-[5-(4-fluorophenyl)-6-isopropyl-pyrrolo[2,3-f]indazol-1-yl]-2,2-dimethyl-propan-1-one C19 (71 g, 188.1 mmol) in CH₂Cl₂ (710 mL) cooled to 0° C. was added 1-iodopyrrolidine-2,5-dione (49 g, 206.9 mmol) over 15 min. The mixture was then allowed to stir at room temperature for 0.5 h. An additional 500 mL of CH₂Cl₂ was added. 1M Na₂S₃O₄ solution (100 mL) and a saturated NaHCO₃ solution (300 mL) were also added. The organic layer was separated, washed with additional sat. NaHCO₃ (300 mL), and then dried over sodium sulfate to afford the product as a brown solid (93 g, 98%). ¹H NMR (300 MHz, Chloroform-d) δ 8.60 (t, J=0.9 Hz, 1H), 8.06 (d, J=0.8 Hz, 1H), 7.40-7.30 (m, 3H), 7.29 (d, J=4.1 Hz, 1H), 7.07 (d, J=0.9 Hz, 1H), 3.18 (p, J=7.2 Hz, 1H), 1.61 (s, 9H), 1.39 (d, J=7.2 Hz, 6H). LCMS m/z 504.2 [M+H]⁺.

### Preparation of Intermediate 49

### Synthesis of 4-[5-(4-fluorophenyl)-6-isopropyl-1H-pyrrolo[2,3-f]indazol-7-yl]-3-methoxy-benzoic acid (49)

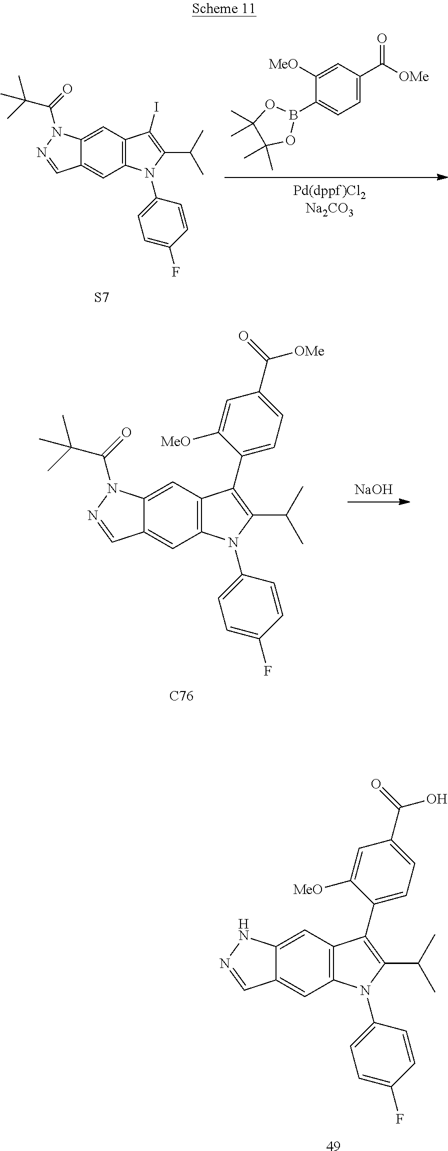

### Step 1. 4-[1-(2,2-dimethylpropanoyl)-5-(4-fluorophenyl)-6-isopropyl-pyrrolo[2,3-f]indazol-7-yl]-3-methoxy-benzoate (C76)

To a solution of 1-[5-(4-fluorophenyl)-7-iodo-6-isopropyl-pyrrolo[2,3-f]indazol-1-yl]-2,2-dimethyl-propan-1-one S7 (4.90 g, 9.50 mmol), methyl 3-methoxy-4-(4,4,5,5-tetramethyl-1,3,2-dioxaborolan-2-yl)benzoate (5.11 g, 17.5 mmol), and Pd(dppf)Cl₂ (604 mg, 0.74 mmol) in 1,4-dioxane (43 mL) was added sodium carbonate (17 mL of 2 M, 34 mmol). The reaction mixture was purged with nitrogen and the solution was stirred at 90° C. for 90 min. Water (100 mL) and dichloromethane (100 mL) were added and the mixture was extracted with dichloromethane (3×100 mL). The organic layers were combined, passed through a phase separator and concentrated in vacuo. Purification by silica gel column chromatography (Eluent: 0-100% dichloromethane in heptane). To a solution of pure material in dichloromethane (150 mL) was added MP-TMT palladium scavenging resin (3.09 g). The suspension was stirred overnight at room temperature. The mixture was filtered, washed with dichloromethane, and concentrated in vacuo to afford the product (2.98 g, 58%). LCMS m/z 542.5 [M+H]⁺.

### Step 2. Synthesis of 4-[5-(4-fluorophenyl)-6-isopropyl-1H-pyrrolo[2,3-f]indazol-7-yl]-3-methoxy-benzoic acid (49)

To a solution of methyl 4-[1-(2,2-dimethylpropanoyl)-5-(4-fluorophenyl)-6-isopropyl-pyrrolo[2,3-f]indazol-7-yl]-3-methoxy-benzoate C76 (1.2 g, 2.15 mmol) in THF (24 mL) and MeOH (12 mL) was added NaOH (12.84 mL of 1 M, 12.84 mmol). The solution was stirred at 50° C. for 1 h. The solvent was evaporated and the crude material was taken up in minimal water. HCl (12.8 mL of 1 M, 12.8 mmol) was added, forming a precipitate. Minimal DMSO was added to the suspension. Purification by reverse phase column chromatography (Eluent: 10-100% acetonitrile in water with 0.2% formic acid modifier) afforded the desired product (1.29 g, 66%). ¹H NMR (400 MHz, DMSO-d₆) δ 13.04 (s, 1H), 12.51 (s, 1H), 7.97 (s, 1H), 7.71-7.66 (m, 2H), 7.64-7.56 (m, 2H), 7.52-7.42 (m, 3H), 7.06 (s, 1H), 6.99 (s, 1H), 3.80 (s, 3H), 2.99 (hept, J=7.6, 6.9 Hz, 1H), 1.08 (d, J=6.9 Hz, 3H), 1.01 (d, J=6.8 Hz, 3H). LCMS m/z 444.4 [M+H]⁺.

### Preparation of 4-(5-(4-fluorophenyl)-6-isopropyl-1,5-dihydropyrrolo[2,3-f]indazol-7-yl)-2-hydroxybenzoic acid (Compound 3)

Compound 49 was reacted with methyl 2-hydroxy-4-(4,4,5,5-tetramethyl-1,3,2-dioxaborolan-2-yl)benzoate to yield 4-(5-(4-fluorophenyl)-6-isopropyl-1,5-dihydropyrrolo[2,3-f]indazol-7-yl)-2-hydroxybenzoic acid (Compound 3).

TABLE 21
¹H NMR data of Compound 3

| | | | ¹H NMR; LCMS |
|---|---|---|---|
| Compound | Method/Product | Boronic acid or ester | m/z [M + H]⁺ |
| 3 | Compound 49 from S7 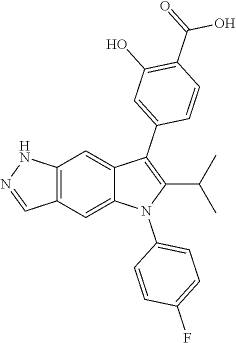 [SMILES: CC(C)c1c(-c2ccc(C(=O)O)c(O)c2)c2cc3[nH]ncc3cc2n1-c1ccc(F)cc1] | 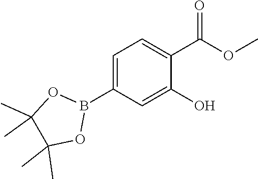 | ¹H NMR (400 MHz, DMSO-d₆) δ 12.60 (s, 1H), 11.39 (s, 1H), 8.00 (d, J = 1.0 Hz, 1H), 7.94 (d, J = 8.1 Hz, 1H), 7.64 − 7.57 (m, 2H), 7.54 − 7.44 (m, 2H), 7.35 (d, J = 1.2 Hz, 1H), 7.10 (dd, J = 8.1, 1,7 Hz, 1H), 7.05 (dd, J = 8.0, 1.4 Hz, 2H), 3.26 − 3.14 (m, 1H), 1.14 (d, J = 7.2 Hz, 6H). LCMS m/z 430.32 [M + H]⁺ |

### Example 5. Assays for Detecting and Measuring AAT Modulator Properties of Solid Forms of Compound 1, and Compounds 2 and 3

A. AAT Function Assay (MSD Assay NL20-SI Cell Line)

Alpha-1 antitrypsin (AAT) is a SERPIN (serine protease inhibitor) that inactivates enzymes by binding to them covalently. This assay measured the amount of functionally active AAT in a sample in the presence of the solid forms of Compound 1 and Compounds 2 and 3 disclosed herein by determining the ability of AAT to form an irreversible complex with human neutrophil Elastase (hNE). In practice, the sample (cell supernatant, blood sample, or other) was incubated with excess hNE to allow AAT-Elastase complex to be formed with all functional AAT in the sample. This complex was then captured to a microplate coated with an anti-AAT antibody. The complex captured to the plate was detected with a labeled anti-Elastase antibody and quantitated using a set of AAT standards spanning the concentration range present in the sample. Meso Scale Discovery (MSD) plate reader, Sulfo-tag labeling, and microplates were used to provide high sensitivity and wide dynamic range.

Materials:

| Reagents/Plates | Concentration |
|---|---|
| Goat anti-human Alpha-1-Antitrypsin | 1 mL @ 1 mg/mL |
| Polyclonal Antibody | |
| Use at 5 μg/mL in phosphate buffered | |
| saline (PBS) | |
| Human Neutrophil Elastase | 100 μg lyophilized |
| Stock at 3.4 μM (0.1 mg + 1 mL PBS) | |
| Working at 1 μg/mL (34 nm) in MSD Assay | |
| buffer (1% bovine serum albumin (BSA)) | |
| Mouse anti-human Neutrophil Elastase | 900 μg/mL |
| Monoclonal Antibody | |
| Sulfo-tagged @ 12:1 using MSD Gold Sulfo- | |
| tag N-hydroxysuccinimide (NHS) ester; use | |
| at 0.45 μg/mL in MSD Assay buffer (1% BSA) | |
| M-AAT (Alpha-1-Antitrypsin) | 5 mg lyophilized |
| MSD Blocker A (BSA) | 250 mL |
| 5% solution in PBS for blocking | |
| 1% solution in PBS for assay buffer | |
| MSD Read Buffer T (4X) with Surfactant | 1 L or 250 mL |
| MSD 384 high bind plates | |
| Polypropylene for dilution 384 well plate | |
| Tissue culture treated black well 384 well plate | |
| INSTRUMENT(S): | |
| Meso Sector S600 | |
| Bravo | |
| Washer dispenser | |
| Multidrop Combi | |

### Assay Protocol

Day 1 Cell Culture
1. Harvest NL20 human bronchial epithelial cells expressing human Z-AAT in OptiMEM™ containing Pen/Strep (P/S)
2. Seed at 16,000 cells/well in 30 μL (384 well plate)
3. Centrifuge plates briefly up to speed (1200 rpm) and place into 37° C. incubator overnight

Day 2: Compound Addition and Coating Plates with Capture Antibody

Compound Addition:
1. Dispense 40 μL of OptiMEM™ (P/S) with doxycycline (1:1000 stock=0.1 μM final) to each well of the compound plate using a multidrop Combi in hood
2. Remove cell plate from incubator, flip/blot and take immediately to Bravo to transfer compounds
3. Return plates to incubator overnight

Coat MSD Plates
1. Dilute capture antibody (Polyclonal Goat anti-AAT) to 5 μg/mL (1:200) in PBS (no BSA).
2. Dispense 25 μL of diluted capture antibody into all wells of MSD 384-well High Bind plate using the Multidrop equipped with a standard cassette.
3. Incubate overnight at 4° C.

Prepare Blocker A (BSA) Solutions
1. Prepare solution of 5% MSD Blocker A (BSA) following the manufacturer's instructions.
2. Further dilute the 5% MSD Blocker A in PBS to 1% (Blocker A) as needed.

Day 3: Run MSD Assay

Block Plates
1. Wash plate 1× with 50 μL Wash buffer (PBS+0.5% Tween 20), and adds 35 μL 5% Block A buffer to block non-specific binding on washer dispenser
2. Rotate plates on shaker for 1 hour at 600 rpm

Prepare M-AAT Standards
1. Dilute M-AAT stock to 1.6 μg/mL in 1% BSA Blocker A (Stock in −70° C.); then prepare 12×1:2 serial dilutions in 1% Blocker A
2. The top starting final concentration on MSD plate is 320 ng/mL. These dilutions correspond to a final concentration of 320, 160, 80, 40, 20, 10, 5, 2.5, 1.25, 0.625, 0.312, 0.156 ng/mL.

Dilution Plate
1. Add 80 μL of 1% Assay buffer to all wells except columns 1/24 (standards) with Multidrop Combi
2. Add diluted standards to columns 1 and 24
3. Centrifuge dilution plates 1200 rpm briefly

Cell Plate
1. Aspirate columns which will have the standards from the cell plates in the hood using 16-pin aspirator

Prepare Human Neutrophil Elastase (hNE)
1. Prepare 1 μg/mL Human Neutrophil Elastase by diluting in 1% Blocker A.
   a. Small 100 μg vial—add 1 mL PBS (100 g/mL)
      i. This can then be diluted 1:100 in 1% Assay Buffer fora final 1 μg/mL concentration

MSD—Add hNE (20 μL/Well)
1. After the MSD plate has blocked for at least 1 hour, wash plate 1× with 50 μL Wash buffer (PBS+0.5% Tween 20) and then add 20 μL hNE to each well

Bravo—Cell Plate—Dilution Plate—MSD Plate

Using the Bravo aspirate 10 μL from the cell plate, transfer to the dilution plate (9-fold dilution)
1. Mix 25 μL 3×, then aspirate 5 μL, transfer to MSD plate (5-fold dilution)
2. Mix 10 μL 3×. Total dilution is 45-fold.
3. Shake plates at 600 rpm for 1.5 hours

Add Functional Detection hNE Antibody
1. Wash plate 1× with wash buffer
2. Add 25 μL Sulfo-tagged anti-Elastase Monoclonal Mouse anti-Elastase) diluted to 0.45 μg/mL (1:2000) in 1% Blocker A into all wells of the functional activity MSD plates using the washer/dispenser
   Note: The dilution required for sufficient signal must be determined for each new lot of labeled antibody.
3. Incubate at RT shaking at 600 rpm for 1 hour.

Final Wash and MSD Imager Read
1. Wash the plate 1×, and add 25 μL of Wash Buffer to the plate.
2. Make 2× Read buffer
3. Remove wash buffer from MSD plate
4. Transfer 35 μL 2× Read Buffer to MSD plate using Bravo and take to MSD to read immediately
Data analysis in MSD Discovery Workbench 4.0 software and EC₅₀ values were determined using Genedata.

B. Biochemical Assay (Z-AAT Elastase Activity Assay)

This assay measured the modulation of the solid forms of Compound 1 disclosed herein on Z-AAT SERPIN activity using purified Z-AAT protein and purified human neutrophil elastase (hNE). Normally, when active monomeric Z-AAT encounters a protease such as trypsin or elastase, it forms a 1:1 covalent “suicide” complex in which both the AAT and protease are irreversibly inactivated. However, compounds binding to Z-AAT can lead to a decrease in SERPIN activity. In such cases, when a protease encounters compound-bound Z-AAT, the protease cleaves and inactivates Z-AAT without itself being inactivated.

Materials

Reagents
PBS buffer (media prep)+0.01% BRIJ35 detergent (Calbiochem catalog #203728)
Opti-MEM media (Fisher 11058-021)
Human neutrophil elastase (hNE, Athens Research #16-14-051200)
   3.4 μM stock (0.1 mg/mL) prepared in 50 mM Na Acetate, pH 5.5, 150 mM NaCl, stored at −80° C.
Elastase substrate V (ES V, fluorescent peptide substrate MeOSuc-Ala-Ala-Pro-Val-AMC, Calbiochem catalog #324740)
   20 mM stock in DMSO, stored at −20° C.
Purified Z-AAT protein from human plasma;
   12.9 μM (0.67 mg/nL) Z-AAT Vertex Cambridge Sample 4942, from patient #061-SSN, stored at −80 C

Plates
Corning 4511 (384 well black low volume)

Instruments
PerkinElmer® EnVision™

Assay Protocol

Pre-Incubation of Z-AAT with Compounds
1. 7.5 μL of Z-AAT (20 nM) was incubated with solid forms of Compound 1 in a GCA plate for 1 hour at room temperature

Addition of hNE
1. 7.5 μL of HNE solution (3 nM in PBS+0.01% BRIJ35) added into GCA plate
2. Incubate plate for 30 minutes to allow Z-AAT/HNE suicide complex formation.

Addition of Substrate and Read Plate on PE Envision
1. 7.5 μL of substrate (300 μM solution of elastase substrate (ES V) in PBS+0.01% BRIJ35) dispensed per well into GCA plate
2. Immediately read on Envision.

### Other Embodiments

This disclosure provides merely exemplary embodiments of the disclosure. One skilled in the art will readily recognize from the disclosure and accompanying figures and claims, that various changes, modifications and variations can be made therein without departing from the spirit and scope of the disclosed subject matter as defined in the following claims.

1. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) neat Form C.

2. The Compound 1 neat Form C according to claim 1, characterized by:
an X-ray powder diffractogram having a signal at 9.4±0.2 degrees two-theta, and a signal at one or more of 15.4±0.2 degrees two-theta, 19.0±0.2 degrees two-theta, and 21.1±0.2 degrees two-theta;
an X-ray powder diffractogram substantially similar to FIG. 1A;
an ¹⁹F ssNMR peak at −107.5±0.2 ppm; and/or
an ¹⁹F ssNMR spectrum substantially similar to FIG. 1B.

3. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Na salt Form A.

4. The Compound 1 Na salt Form A according to claim 3, characterized by:
an X-ray powder diffractogram having a signal at at least one of 7.3±0.2 degrees two-theta and 11.6±0.2 degrees two-theta; and/or
an X-ray powder diffractogram substantially similar to FIG. 2A.

5. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Na salt Form B.

6. The Compound 1 Na salt Form B according to claim 5, characterized by:
an X-ray powder diffractogram having signals at 3.1±0.2 degrees two-theta and 8.9±0.2 degrees two-theta; and/or
an X-ray powder diffractogram substantially similar to FIG. 3A.

7. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Na salt Form C.

8. The Compound 1 Na salt Form C according to claim 7, characterized by:
an X-ray powder diffractogram having signals at 19.7±0.2 degrees two-theta, 9.2±0.2 degrees two-theta, and 13.3±0.2 degrees two-theta; and/or
an X-ray powder diffractogram substantially similar to FIG. 4A; and/or
a ¹³C ssNMR peak at one or more of 138.1±0.2 ppm, 121.5±0.2 ppm, 117.4±0.2 ppm, 115.2±0.2 ppm, 36.7±0.2 ppm, and 32.1±0.2 ppm; and/or
a ¹³C ssNMR spectrum substantially similar to FIG. 4B; and/or
a ²³Na ssNMR peak at −11.2±0.2 ppm and/or −14.0±0.2 ppm; and/or
a ²³Na ssNMR spectrum substantially similar to FIG. 4C.

9. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Na salt Form D.

10. The Compound 1 Na salt Form D according to claim 9, characterized by:
an X-ray powder diffractogram having signals 3.5±0.2 degrees two-theta and 16.2±0.2 degrees two-theta; and/or
an X-ray powder diffractogram substantially similar to FIG. 5A; and/or
a ¹³C ssNMR peak at one or more of 175.8±0.2 ppm, 142.0±0.2 ppm, 134.0±0.2 ppm, 119.3±0.2 ppm, 97.9±0.2 ppm, 67.7±0.2 ppm, and 37.2±0.2 ppm; and/or
a ¹³C ssNMR spectrum substantially similar to FIG. 5B; and/or
a ²³Na ssNMR peak at one or more of 5.3±0.2 ppm, 2.1±0.2 ppm, −5.0±0.2 ppm, and −6.3±0.2 ppm; and/or
a ²³Na ssNMR spectrum substantially similar to FIG. 5C.

11. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) Ca salt Form A.

12. The Compound 1 Ca salt Form A according to claim 11, characterized by:
an X-ray powder diffractogram having signals at 17.9±0.2 degrees two-theta and at least one of 11.7±0.2 degrees two-theta and 20.5±0.2 degrees two-theta; and/or
an X-ray powder diffractogram substantially similar to FIG. 6A.

13. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) HCl salt Form A.

14. The Compound 1 HCl salt Form A according to claim 13, characterized by:
an X-ray powder diffractogram having a signal at one or more of 8.1±0.2 degrees two-theta, 7.8±0.2 degrees two-theta, and 9.0±0.2 degrees two-theta; and/or
an X-ray powder diffractogram substantially similar to FIG. 7A.

15. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) DMSO solvate Form A.

16. The Compound 1 DMSO solvate Form A according to claim 15, characterized by:
an X-ray powder diffractogram having a signal at one or more of 9.9±0.2 degrees two-theta, 19.1±0.2 degrees two-theta, and 19.8±0.2 degrees two-theta; and/or
an X-ray powder diffractogram substantially similar to FIG. 8A.

17. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) EtOH solvate Form A.

18. The Compound 1 EtOH solvate Form A according to claim 17, characterized by:
an X-ray powder diffractogram having a signal at one or more of 20.2±0.2 degrees two-theta, 20.7±0.2 degrees two-theta, and 23.4±0.2 degrees two-theta; and/or
an X-ray powder diffractogram substantially similar to FIG. 9A; and/or
a ¹³C ssNMR peak at one or more of 126.6±0.2 ppm, 111.5±0.2 ppm, 57.9±0.2 ppm, 34.4±0.2 ppm, 27.9±0.2 ppm, and 19.0±0.2 ppm; and/or
a ¹³C ssNMR spectrum substantially similar to FIG. 9B.

19. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) tartrate salt or cocrystal Form A.

20. The Compound 1 tartrate salt or cocrystal Form A according to claim 19, characterized by:
an X-ray powder diffractogram having signals at 19.0±0.2 degrees two-theta, 19.6±0.2 degrees two-theta, and 20.5±0.2 degrees two-theta; and/or
an X-ray powder diffractogram substantially similar to FIG. 10A.

21. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) tartrate salt or cocrystal Form B.

22. The Compound 1 tartrate salt or cocrystal Form B according to claim 21, characterized by:
an X-ray powder diffractogram having signals at 8.9±0.2 degrees two-theta, 17.8±0.2 degrees two-theta, and 22.7±0.2 degrees two-theta; and/or
an X-ray powder diffractogram substantially similar to FIG. 11A.

23. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) tartrate salt or cocrystal Form C.

24. The Compound 1 tartrate salt or cocrystal Form C according to claim 23, characterized by:
an X-ray powder diffractogram having signals at 12.4±0.2 degrees two-theta, 13.3±0.2 degrees two-theta, and 18.5±0.2 degrees two-theta; and/or
an X-ray powder diffractogram substantially similar to FIG. 12A.

25. A substantially pure crystalline 4-(5-(4-fluorophenyl)-6-(tetrahydro-2H-pyran-4-yl)-1,5-dihydropyrrolo[2,3-f]indazol-7-yl)benzoic acid (Compound 1) tartrate salt or cocrystal Form D.

26. The Compound 1 tartrate salt or cocrystal Form D according to claim 25, characterized by an X-ray powder diffractogram having a signal at one or more of 13.8±0.2 degrees two-theta, 14.8±0.2 degrees two-theta, and 25.2±0.2 degrees two-theta.

27. A solid dispersion comprising a solid form of 4-(5-(4-fluorophenyl)-6-(tetrahydro-2H-pyran-4-yl)-1,5-dihydropyrrolo[2,3-f]indazol-7-yl)benzoic acid (Compound 1) or a salt, solvate, or cocrystal thereof and a polymer carrier; wherein the solid dispersion is prepared by dissolving the solid form of Compound 1 or a salt, solvate, or cocrystal thereof in a solvent system comprising a first organic solvent, a second organic solvent, and optionally water; wherein:
when water is absent from the solvent system, the volume ratio of the first organic solvent to the second organic solvent is between about 55/45 v/v and about 90/10 v/v; and
when water is present in the solvent system, the weight ratio of the first organic solvent to the second organic solvent and to water is between about 55/35/10 w/w and about 80/10/10 w/w or about 55/35/10 w/w and about 65/34.5/0.5 w/w; wherein when the weight ratio of the first organic solvent to the second organic solvent and to water is about 55/35/10 w/w, the solid dispersion comprises greater than about 50% w/w of the solid form of Compound 1 or a salt, solvate, or cocrystal thereof.

28. The solid dispersion according to claim 27, wherein the solid dispersion comprises no lower than about 50% w/w of the solid form of Compound 1 or a salt, solvate, or cocrystal thereof; and wherein when the weight ratio of the first organic solvent to the second organic solvent and to water is about 55/35/10 w/w, the solid dispersion comprises higher than about 50% w/w of the solid form of Compound 1 or a salt, solvate, or cocrystal thereof.

29. The solid dispersion according to claim 27 or 28, wherein:
the polymer is PVP-VA or HPMCAS-H; and/or
the first organic solvent is selected from DCM, THF, and Me-THF; and/or
the second organic solvent is MeOH or EtOH.

30. The solid dispersion according to any one of claims 27-29, wherein the solid dispersion is a spray dried dispersion.

31. A compound represented by one of the following structural formulae: 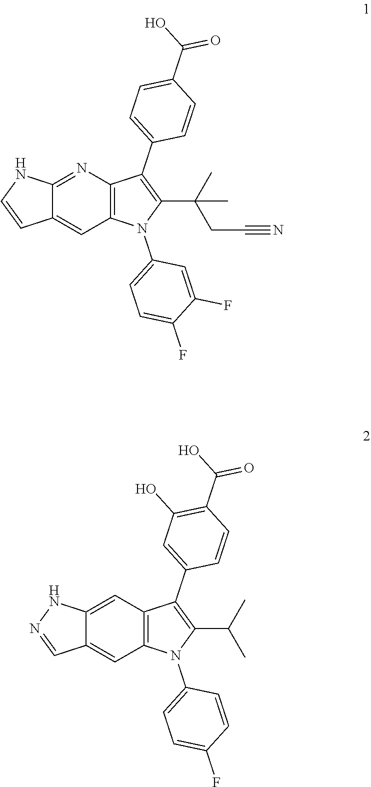 [SMILES: CC(C)(CC#N)c1c(-c2ccc(C(=O)O)cc2)c2nc3[nH]ccc3cc2n1-c1ccc(F)c(F)c1 ; CC(C)c1c(-c2ccc(C(=O)O)c(O)c2)c2cc3[nH]ncc3cc2n1-c1ccc(F)cc1]
a tautomer thereof, a deuterated derivative of that compound or tautomer, or a pharmaceutically acceptable salt of the foregoing.

32. A pharmaceutical composition comprising the Compound 1 neat Form C according to claim 1 or 2; or the Compound 1 Na salt Form A according to claim 3 or 4; or the Compound 1 Na salt Form B according to claim 5 or 6; or the Compound 1 Na salt Form C according to claim 7 or 8; or the Compound 1 Na salt Form D according to claim 9 or 10; or the Compound 1 Ca salt Form A according to claim 11 or 12; or the Compound HCl salt Form A according to claim 13 or 14; or the Compound 1 DMSO solvate Form A according to claim 15 or 16; or the Compound 1 EtOH solvate Form A according to claim 17 or 18; or the Compound 1 tartrate salt or cocrystal Form A according to claim 19 or 20; or the Compound 1 tartrate salt or cocrystal Form B according to claim 21 or 22; or the Compound 1 tartrate salt or cocrystal Form C according to claim 23 or 24; or the Compound 1 tartrate salt or cocrystal Form D according to claim 25 or 26; or the solid dispersion according to any one of claims 27-30; or the compound according to claim 31 or a tautomer thereof, a deuterated derivative of that compound or tautomer, or a pharmaceutically acceptable salt of the foregoing; and a pharmaceutically acceptable carrier.

33. A method of treating alpha-1 antitrypsin deficiency comprising administering to a patient in need thereof the Compound 1 neat Form C according to claim 1 or 2; or the Compound 1 Na salt Form A according to claim 3 or 4; or the Compound 1 Na salt Form B according to claim 5 or 6; or the Compound 1 Na salt Form C according to claim 7 or 8; or the Compound 1 Na salt Form D according to claim 9 or 10; or the Compound 1 Ca salt Form A according to claim 11 or 12; or the Compound HCl salt Form A according to claim 13 or 14; or the Compound 1 DMSO solvate Form A according to claim 15 or 16; or the Compound 1 EtOH solvate Form A according to claim 17 or 18; or the Compound 1 tartrate salt or cocrystal Form A according to claim 19 or 20; or the Compound 1 tartrate salt or cocrystal Form B according to claim 21 or 22; or the Compound 1 tartrate salt or cocrystal Form C according to claim 23 or 24; or the Compound 1 tartrate salt or cocrystal Form D according to claim 25 or 26; or the solid dispersion according to any one of claims 27-30; or the compound according to claim 31 or a tautomer thereof, a deuterated derivative of that compound or tautomer, or a pharmaceutically acceptable salt of the foregoing; and a pharmaceutically acceptable carrier; or the pharmaceutical composition according to claim 32.

34. The method according to claim 33, wherein:
the patient has a Z mutation in alpha-1 antitrypsin; or
the patient has an SZ mutation in alpha-1 antitrypsin; or
the patient is homozygous for Z-mutations in alpha-1 antitrypsin.

35. A method of modulating alpha-1 antitrypsin activity comprising contacting said alpha-1-antitrypsin with the Compound 1 neat Form C according to claim 1 or 2; or the Compound 1 Na salt Form A according to claim 3 or 4; or the Compound 1 Na salt Form B according to claim 5 or 6; or the Compound 1 Na salt Form C according to claim 7 or 8; or the Compound 1 Na salt Form D according to claim 9 or 10; or the Compound 1 Ca salt Form A according to claim 11 or 12; or the Compound HCl salt Form A according to claim 13 or 14; or the Compound 1 DMSO solvate Form A according to claim 15 or 16; or the Compound 1 EtOH solvate Form A according to claim 17 or 18; or the Compound 1 tartrate salt or cocrystal Form A according to claim 19 or 20; or the Compound 1 tartrate salt or cocrystal Form B according to claim 21 or 22; or the Compound 1 tartrate salt or cocrystal Form C according to claim 23 or 24; or the Compound 1 tartrate salt or cocrystal Form D according to claim 25 or 26; or the solid dispersion according to any one of claims 27-30; or the compound according to claim 31 or a tautomer thereof, a deuterated derivative of that compound or tautomer, or a pharmaceutically acceptable salt of the foregoing; and a pharmaceutically acceptable carrier; or the pharmaceutical composition according to claim 32.
